(19) United States
(12) Patent Application Publication
(10) Pub. No.: US 2023/0172880 A1
(43) Pub. Date: Jun. 8, 2023

(54) FAST-ACTING TOPICAL ANESTHETIC FORMULATIONS

(71) Applicant: Achelon, Inc., Redondo Beach, CA (US)

(72) Inventors: Manju Chacko Dawkins, Redondo Beach, CA (US); John M. Newsam, La Jolla, CA (US); Avadhesh Kushwaha, San Diego, CA (US); Edward T. Kisak, San Diego, CA (US)

(21) Appl. No.: 17/997,515

(22) PCT Filed: Apr. 28, 2021
(86) PCT No.: PCT/US2021/029719

§ 371 Date: Oct. 28, 2022

Related U.S. Application Data
(60) Provisional application No. 63/019,042, filed on May 1, 2020.
(60) Provisional application No. 63/139,224, filed on Jan. 19, 2021.

(51) Int. Cl. A61K 31/167; A61K 31/015; A61K 9/00; A61K 47/14; A61K 47/10; A61K 47/38; A61K 9/70; A61P 23/02

(52) U.S. Cl. CPC A61K 31/167; A61K 9/0014; A61K 9/7084; A61K 31/015; A61K 47/10; A61K 47/14; A61K 47/38; A61P 23/02

(57) ABSTRACT

The present invention relates to formulations of a local anesthetic that provide prompt local anesthesia following topical administration, teaching formulation compositions suitable for administration to the skin that provide onsets of less than one hour. Also disclosed are methods of making the formulations and methods of preventing pain in a subject.

### CROSS-REFERENCE TO RELATED APPLICATIONS

This application claims the benefit of priority of U.S. Provisional Pat. Application No. 63/019,042, filed May 1, 2020, and U.S. Provisional Pat. Application No. 63/139,224, filed Jan. 19, 2021, each of which is hereby expressly incorporated by reference in its entirety.

### FIELD

The present disclosure relates to formulations and devices suitable for providing rapid local anesthesia. More specifically, the present disclosure relates to formulations including a local anesthetic such as lidocaine and other compounds, molecules, or excipients that improve desirable characteristics such as solubility, bioavailability, efficacy, tolerability, stability, or improved dosages. The formulations may be made into a spray, a cream, a lotion, an emulsion, a microemulsion, a gel, a lacquer, an ointment, a solution, a patch, film or mask, or other topical application. Further disclosed are methods for using such formulations for providing rapid local anesthesia.

### BACKGROUND

A local anesthetic (“LA”) is a medication that moderates or eliminates the sensation of pain in the tissues that are local to the site of medication application. Clinical local anesthetics belong to one of two classes: aminoesters and aminoamides. Local anesthetic drugs act mainly by inhibiting sodium influx through sodium-specific ion channels in the neuronal cell membrane, in particular voltage-gated sodium channels. When the influx of sodium is interrupted, an action potential cannot develop and signal conduction is inhibited. The receptor site is thought to be located at the cytoplasmic or cell-interior portion of the sodium channel. Local anesthetic drugs bind more readily to sodium channels in an activated state. Onset of neuronal blockade is then faster in rapidly firing neurons, termed a state-dependent blockade.

### SUMMARY

The present disclosure relates to formulations of a local anesthetic that provide prompt local anesthesia following topical administration and methods of using such formulations.

Several embodiments disclosed herein provide a topical formulation that comprises an aminoamide or an aminoester local anesthetic with an anesthesia onset of less than one hour.

Several embodiments disclosed herein provide a topical formulation that comprises an aminoamide or an aminoester local anesthetic with an anesthesia onset of less than thirty minutes.

Several embodiments disclosed herein provide a topical formulation that comprises lidocaine.

Several embodiments disclosed herein provide a topical formulation that further comprises a C2-C4 monohydric alcohol and a monohydric C 14-C20 alcohol.

Several embodiments disclosed herein provide a topical formulation that further comprises an isopropyl ester of a C14-C18 fatty acid.

Several embodiments disclosed herein provide a topical formulation that further comprises limonene.

Several embodiments disclosed herein provide a topical formulation that further comprises a polyethylene glycol dodecyl ether.

Several embodiments disclosed herein provide a topical formulation that comprises lidocaine, a C2-C4 monohydric alcohol, limonene, an isopropyl ester of a C 14-C 18 fatty acid, and a monohydric C14-C20 alcohol.

Several embodiments disclosed herein provide a topical formulation that comprises lidocaine, ethanol, limonene, isopropyl myristate, isopropyl palmitate, and isostearyl alcohol.

Several embodiments disclosed herein provide a topical formulation that further comprises a polyethylene glycol dodecyl ether.

Several embodiments disclosed herein provide a topical formulation further comprising a thickening agent.

Several embodiments disclosed herein provide a topical formulation further comprising an emollient, a preservative, or mixtures thereof.

Several embodiments disclosed herein provide a topical formulation that comprises lidocaine, a C2-C4 monohydric alcohol, limonene, an isopropyl ester of a C 14-C 18 fatty acid, a monohydric C14-C20 alcohol, and a pressure-sensitive adhesive.

Several embodiments disclosed herein provide a use of the topical formulation of the application for administration to a subject in need thereof.

Several embodiments disclosed herein provide a topical formulation comprising (i) at least one active agent, (ii) a C2-C4 monohydric alcohol, (iii) limonene, (iv) an isopropyl ester of a C14-C18 fatty acid, and (v) a monohydric C14-C20 alcohol.

Several embodiments disclosed herein provide a topical formulation of local anesthetic comprising one or more of (i) at least one active agent, (ii) a C2-C4 monohydric alcohol, (iii) limonene, (iv) an isopropyl ester of a C14-C18 fatty acid, and (v) a monohydric C14-C20 alcohol that is suitable for immediate sale in the United States as an over the counter drug product subject to the provisions of an FDA monograph.

Certain features are briefly described in the following non-limiting numbered embodiments:

1. A topical formulation comprising:
lidocaine present in an amount of 1% to 10% or about 1% to about 10% w/w;
limonene present in an amount of 5% to 20% or about 5% to about 20% w/w; and
at least one C2-C4 monohydric alcohol present in an amount of 40% to 60% or about 40% to about 60% w/w.

2. The topical formulation of alternative 1, wherein the at least one C2-C4 monohydric alcohol comprises ethanol.

3. The topical formulation of alternative 1 or 2, wherein the at least one C2-C4 monohydric alcohol is ethanol.

4. The topical formulation of any one of alternatives 1-3, wherein the lidocaine is present in an amount of 4% or about 4% w/w.

5. The topical formulation of any one of alternatives 1-4, wherein the limonene is present in an amount of 10% or about 10% w/w.

6. The topical formulation of any one of alternatives 1-5, wherein the at least one C2-C4 monohydric alcohol is present in an amount of 51% or about 51% w/w.

7. The topical formulation of any one of alternatives 1-6, further comprising at least one isopropyl ester of a C14-C18 fatty acid.

8. The topical formulation of alternative 7, wherein the at least one isopropyl ester of a C14-C18 fatty acid is present in an amount of 10% to 30% or about 10% to about 30% w/w.

9. The topical formulation of alternative 7 or 8, wherein the at least one isopropyl ester of a C14-C18 fatty acid comprises isopropyl palmitate or isopropyl myristate, or both.

10. The topical formulation of alternative 9, wherein the isopropyl palmitate and the isopropyl myristate are each present in an amount of 5% to 15% or about 5% to about 15% w/w.

11. The topical formulation of alternative 9 or 10, wherein the isopropyl palmitate is present in an amount of 7% or about 7% w/w.

12. The topical formulation of any one of alternatives 9-11, wherein the isopropyl myristate is present in an amount of 11% or about 11% w/w.

13. The topical formulation of any one of alternatives 9-12, wherein the lidocaine is present in an amount of 4% or about 4% w/w, the limonene is present in an amount of 10% or about 10% w/w, the at least one C2-C4 monohydric alcohol is present in an amount of 51% or about 51% w/w, the isopropyl palmitate is present in an amount of 7% or about 7% w/w, and the isopropyl myristate is present in an amount of 11% or about 11% w/w.

14. The topical formulation of any one of alternatives 1-13, further comprising at least one monohydric C14-C20 alcohol.

15. The topical formulation of alternative 14, wherein the at least one monohydric C14-C20 alcohol is present in an amount of 5% to 15% or about 5% to about 15% w/w.

16. The topical formulation of alternative 14 or 15, wherein the at least one monohydric C14-C20 alcohol comprises isostearyl alcohol.

17. The topical formulation of any one of alternatives 14-16, wherein the at least one monohydric C14-C20 alcohol is isostearyl alcohol.

18. The topical formulation of alternative 16 or 17, wherein the isostearyl alcohol is present in an amount of 10% or about 10% w/w.

19. The topical formulation of any one of alternatives 14-18, wherein the lidocaine is present in an amount of 4% or about 4% w/w, the limonene is present in an amount of 10% or about 10% w/w, the at least one C2-C4 monohydric alcohol is present in an amount of 51% or about 51% w/w, the isopropyl palmitate is present in an amount of 7% or about 7% w/w, the isopropyl myristate is present in an amount of 11% or about 11% w/w, and the at least one monohydric C14-C20 alcohol is present in an amount of 10% or about 10% w/w.

20. The topical formulation of any one of alternatives 1-19, further comprising at least one polyalkylene glycol alkyl ether.

21. The topical formulation of alternative 20, wherein the at least one polyalkylene glycol alkyl ether is present in an amount of 0% to 10% or about 0% to about 10%.

22. The topical formulation of alternative 20 or 21, wherein the at least one polyalkylene glycol alkyl ether comprises polyethylene glycol dodecyl ether.

23. The topical formulation of any one of alternatives 20-22, wherein the at least one polyalkylene glycol alkyl ether is polyethylene glycol dodecyl ether.

24. The topical formulation of alternative 22 or 23, wherein the polyethylene glycol dodecyl ether is present in an amount of 5% or about 5% w/w.

25. The topical formulation of any one of alternatives 20-24, wherein the lidocaine is present in an amount of 4% or about 4% w/w, the limonene is present in an amount of 10% or about 10% w/w, the at least one C2-C4 monohydric alcohol is present in an amount of 51% or about 51% w/w, the isopropyl palmitate is present in an amount of 7% or about 7% w/w, the isopropyl myristate is present in an amount of 11% or about 11% w/w, the at least one monohydric C14-C20 alcohol is present in an amount of 10% or about 10% w/w, and the at least one polyalkylene glycol alkyl ether is present in an amount of 5% or about 5% w/w.

26. The topical formulation of any one of alternatives 1-25, further comprising a cellulosic thickening agent.

27. The topical formulation of alternative 26, wherein the cellulosic thickening agent is present in an amount of 0% to 5% or about 0% to about 5%.

28. The topical formulation of alternative 25 or 26, wherein the cellulosic thickening agent comprises hydroxypropyl cellulose.

29. The topical formulation of alternative 28, wherein the hydroxypropyl cellulose is present in an amount of 2% or about 2%.

30. The topical formulation of any one of alternatives 26-29, wherein the lidocaine is present in an amount of 4% or about 4% w/w, the limonene is present in an amount of 10% or about 10% w/w, the at least one C2-C4 monohydric alcohol is present in an amount of 51% or about 51% w/w, the isopropyl palmitate is present in an amount of 7% or about 7% w/w, the isopropyl myristate is present in an amount of 11% or about 11% w/w, the at least one monohydric C14-C20 alcohol is present in an amount of 10% or about 10% w/w, the at least one polyalkylene glycol alkyl ether is present in an amount of 5% or about 5% w/w, and the cellulosic thickening agent is present in an amount of 2% or about 2%.

31. A topical formulation comprising:
lidocaine present in an amount of 1% to 10% or about 1% to about 10% w/w;
a pressure sensitive adhesive present in an amount of 70% to 90% or about 70% to about 90% w/w;
a molecular penetration enhancer present in an amount of 0% to 5% or about 0% to about 5% w/w; and
at least one polyalkylene glycol alkyl ether present in an amount of 0% to 5% or about 0% to about 5% w/w.

32. The topical formulation of alternative 31, wherein the lidocaine is present in an amount of 4% or about 4% w/w.

33. The topical formulation of alternative 31 or 32, wherein the pressure sensitive adhesive is present in an amount of 83% or about 83% w/w.

34. The topical formulation of any one of alternatives 31-33, wherein the pressure sensitive adhesive comprises DURO-TAK 87-4098.

35. The topical formulation of any one of alternatives 31-34, wherein the molecular penetration enhancer is present in an amount of 3% or about 3% w/w.

36. The topical formulation of any one of alternatives 31-35, wherein the molecular penetration enhancer comprises levulinic acid.

37. The topical formulation of any one of alternatives 31-36, wherein the at least one polyalkylene glycol alkyl ether is present in an amount of 2% or about 2%.

38. The topical formulation of any one of alternatives 31-37, wherein the at least one polyalkylene glycol alkyl ether comprises polyethylene glycol dodecyl ether.

39. The topical formulation of any one of alternatives 31-38, wherein the at least one polyalkylene glycol alkyl ether is polyethylene glycol dodecyl ether.

40. The topical formulation of any one of alternatives 31-39, further comprising at least one isopropyl ester of a C14-C18 fatty acid.

41. The topical formulation of alternative 40, wherein the at least one isopropyl ester of a C14-C18 fatty acid is present in an amount of 5% to 10% or about 5% to about 10% w/w.

42. The topical formulation of alternative 40 or 41, wherein the at least one isopropyl ester of a C14-C18 fatty acid comprises isopropyl palmitate or isopropyl myristate, or both.

43. The topical formulation of alternative 42, wherein the isopropyl palmitate and the isopropyl myristate are each present in an amount of 2.5% to 5% or about 2.5% to about 5% w/w.

44. The topical formulation of alternative 42 or 43, wherein the isopropyl palmitate or the isopropyl myristate, or both, are present in an amount of 4% or about 4% w/w.

45. The topical formulation of any one of alternatives 31-44, wherein the lidocaine is present in an amount of 4% or about 4% w/w, the pressure sensitive adhesive is present in an amount of 83% or about 83% w/w, the molecular penetration enhancer is present in an amount of 3% or about 3% w/w, the polyalkylene glycol alkyl ether is present in an amount of 2% or about 2% w/w, the isopropyl palmitate is present in an amount of 4% or about 4% w/w, and the isopropyl myristate is present in an amount of 4% or about 4% w/w.

46. A method of preventing local pain in a subject, comprising the topical administration to said subject of a therapeutically effective amount of the topical formulation of any one of alternatives 1-45.

47. A device suitable for providing rapid local anesthesia comprising an annulus of pressure sensitive adhesive, within the inner circumference of which is provided the topical formulation of any one of alternatives 1-45.

48. The device of alternative 47, further comprising a backing film on which the topical formulation is applied, wherein the backing film covers the inner circumference of the annulus of pressure sensitive adhesive.

49. The device of alternative 48, wherein the backing film is intended to be removed after application of the device such that the inner circumference of the annulus of pressure sensitive adhesive is exposed to allow access with a needle.

50. The device of any one of alternatives 47-49, wherein the topical formulation is provided as a single dose packet.

51. A kit for use in anesthetizing a target area of skin of a subject, the kit comprising:
(i) a predetermined dose of a topical anesthetic; and
(ii) an annulus of pressure sensitive adhesive comprising an outer perimeter that adheres to the skin of the subject, and an open center region in which the skin is exposed.

52. The kit of alternative 51, further comprising (iii) an occlusive, waterproof dressing or a backing film to cover the patch and target area.

53. The kit of alternative 51 or 52, wherein the topical anesthetic comprises a topical formulation of any one of alternatives 1-45.

54. The kit of any one of alternatives 51-53, wherein the topical anesthetic comprises lidocaine and wherein the lidocaine is optionally in gel format.

55. The kit of any one of alternatives 51-54, wherein the annulus of pressure sensitive adhesive comprises a silicone ring.

56. The kit of any one of alternatives 51-54, wherein the predetermined dose of the topical anesthetic is provided as a single packet (e.g., single use).

57. A topical formulation comprising
lidocaine, wherein the lidocaine is present in an amount of 1% to 10% or about 1% to about 10% w/w;
limonene, wherein the limonene is present in an amount of 5% to 20% or about 5% to about 20% w/w;
DMSO, wherein the DMSO is present in an amount of 40% to 70% or about 40% to about 70% w/w; and
at least one C2-C4 monohydric alcohol,
wherein the at least one C2-C4 monohydric alcohol comprises ethanol, and wherein the ethanol is present in an amount of 10% to 50% or about 10% to 50% w/w.

58. The topical formulation of alternative 57, wherein
(i) lidocaine is in an amount of 4% or about 4%;
(ii) limonene is in an amount of 10% or about 10%;
(iii) DMSO is in an amount of 66% or about 66%; and
(iv) ethanol is in an amount of 20% or about 20%.

59. The topical formulation of alternative 57, wherein
(i) lidocaine is in an amount of 4% or about 4%;
(ii) limonene is in an amount of 10% or about 10%;
(iii) DMSO is in an amount of 45% or about 45%; and
(iv) ethanol is in an amount of 41% or about 41%.

60. The topical formulation of alternative 57, further comprising at least one isopropyl ester of a C14-C18 fatty acid.

61. The topical formulation of alternative 60, wherein the at least one isopropyl ester of a C14-C18 fatty acid comprises isopropyl myristate, or isopropyl palmitate, or both.

62. The topical formulation of alternative 61, wherein isopropyl myristate and isopropyl palmitate are each in an amount of 0% to 15% or about 0% to about 15%.

63. The topical formulation of alternative 62, wherein
(i) lidocaine is in an amount of 4% or about 4%;
(ii) limonene is in an amount of 10% or about 10%;
(iii) DMSO is in an amount of 45% or about 45%;
(iv) ethanol is in an amount of 31% or about 31%;
(v) isopropyl myristate is in an amount of 10% or about 10%; and
(vi) isopropyl palmitate is in an amount of 0% or about 0%.

64. The topical formulation of alternative 62, wherein
(i) lidocaine is in an amount of 4% or about 4%;
(ii) limonene is in an amount of 10% or about 10%;
(iii) DMSO is in an amount of 45% or about 45%;
(iv) ethanol is in an amount of 31% or about 31%;
(v) isopropyl myristate is in an amount of 0% or about 0%; and
(vi) isopropyl palmitate is in an amount of 10% or about 10%.

65. The topical formulation of alternative 60, further comprising at least one monohydric C14-C20 alcohol.

66. The topical formulation of alternative 65, wherein the at least one monohydric C14-C20 alcohol comprises isostearyl alcohol.

67. The topical formulation of any one of alternatives 57-66, further comprising polyethylene glycol dodecyl ether.

68. A method of preventing local pain in a subject, said method comprising the topical administration to said subject of a therapeutically effective amount of the formulation of any one of Alternatives 57-67.

69. A device suitable for providing rapid local anesthesia comprising an annulus of pressure-sensitive adhesive, within the inner circumference of which is provided the topical formulation of any one of Alternatives 57-67.

70. A topical formulation comprising lidocaine, at least one C2-C4 monohydric alcohol, limonene, at least one isopropyl ester of a C14-C18 fatty acid, and at least one monohydric C14-C20 alcohol.

71. The topical formulation of Alternative 70, wherein the at least one C2-C4 monohydric alcohol comprises ethanol.

72. The topical formulation of Alternative 70, wherein the at least one isopropyl ester of the C14-C18 fatty acid comprises isopropyl myristate.

73. The topical formulation of Alternative 70, wherein the at least one isopropyl ester of the C14-C18 fatty acid comprises isopropyl palmitate.

74. The topical formulation of Alternative 70, wherein the at least one isopropyl ester of the C14-C18 fatty acid comprises both isopropyl myristate and isopropyl palmitate.

75. The topical formulation of Alternative 70, wherein the at least one monohydric C14-C20 alcohol comprises isostearyl alcohol.

76. The topical formulation of any one of Alternatives 70-75, further comprising polyethylene glycol dodecyl ether.

77. A method of preventing local pain in a subject, said method comprising the topical administration to said subject of a therapeutically effective amount of the formulation of any one of Alternatives 70-76.

78. A device suitable for providing rapid local anesthesia comprising an annulus of pressure-sensitive adhesive, within the inner circumference of which is provided the topical formulation of any one of Alternatives 70-76.

79. A formulation suitable for topical administration, comprising:
(i) at least one active agent;
(ii) at least one C2-C4 monohydric alcohol;
(iii) limonene;
(iv) the isopropyl esters of at least two C14-C18 fatty acids; and
(v) at least one monohydric C14-C20 alcohol.

Several embodiments disclosed herein relate to a kit for use in anesthetizing a target area of tissue, the kit comprising:
a predetermined dose of a topical anesthetic;
a self-adhesive patch comprising an outer perimeter which adheres to the skin and an open center region in which skin is exposed;
an occlusive, waterproof dressing to cover the patch and target area.

In several embodiments, the topical anesthetic comprises a formulation (e.g., a fast acting anesthetic formulation) according to embodiments disclosed herein. In several embodiments, the topical anesthetic comprises lidocaine, wherein the lidocaine is optionally in gel format. In several embodiments, the self-adhesive patch comprises a silicone ring.

### BRIEF DESCRIPTION OF THE DRAWINGS

In addition to the features described above, additional features and variations will be readily apparent from the following descriptions of the drawings and non-limiting embodiments. It is to be understood that these drawings depict non-limiting embodiments and are not intended to be limiting in scope.

The embodiments of the application will now be described in greater detail with reference to the attached drawings in which:

FIG. 1 illustrates cumulative amounts of lidocaine that are found to be retained in porcine skin 60 minutes following topical administration of the Example 1 formulations. All retention amounts are provided as µ g per cm² of administration area.

FIG. 2 illustrates cumulative amounts of lidocaine that are found to be retained in porcine skin 60 minutes following topical administration of the Example 2 formulations. All retention amounts are provided as µ g per cm² of administration area.

FIG. 3 illustrates cumulative amounts of lidocaine that are found to be retained in porcine skin 60 minutes following topical administration of the Example 3 formulations. All retention amounts are provided as µ g per cm² of administration area.

FIG. 4 illustrates cumulative amounts of lidocaine that are found to be retained in porcine skin 60 minutes following topical administration of the Example 4 formulations. All retention amounts are provided as µ g per cm² of administration area.

FIG. 5 illustrates cumulative amounts of lidocaine that are found to be retained in porcine skin 60 minutes following topical administration of the Example 5 formulations. All retention amounts are provided as µ g per cm² of administration area.

FIG. 6 illustrates cumulative amounts of lidocaine that are found to be retained in porcine skin 30 minutes following topical administration of the Example 6 formulations. All retention amounts are provided as µ g per cm² of administration area.

FIG. 7 illustrates cumulative amounts of lidocaine that are found to be in porcine skin 30 minutes following topical administration of the Example 7 formulations. All retention amounts are provided as µ g per cm² of administration area.

FIG. 8 illustrates cumulative amounts of lidocaine that are found to be retained in porcine skin 30 minutes following topical administration of the Example 8 formulations. All retention amounts are provided as µ g per cm² of administration area.

FIG. 9 illustrates cumulative amounts of lidocaine that are found to be retained in within the epidermal and dermal compartments of human cadaver at 30 mins following topical administration of the Example 9 formulations. All retention amounts are provided as µ g per cm² of administration area.

FIG. 10 illustrates cumulative amounts of lidocaine that are found to be retained within the epidermal and dermal compartments of human cadaver at 30 minutes following topical administration of the Example 10 formulations. All retention amounts are provided as µ g per cm² of administration area.

FIG. 11 illustrates cumulative amounts of lidocaine that are found to be retained in human cadaver skin (the combined epidermal and dermal tissue) at 30 mins following topical administration of the Example 11 formulations. All retention amounts are provided as µ g per cm² of administration area.

FIG. 12 illustrates cumulative amounts of lidocaine that are found to be retained within human cadaver skin (the combined epidermal and dermal tissue) at 30 minutes following topical administration of the Example 12 formulations. All retention amounts are provided as µ g per cm² of administration area.

FIG. 13 show an example of a kit comprising a topical patch with an annulus of pressure-sensitive adhesive and a topical formulation in use according to several embodiments disclosed herein.

FIG. 14 shows a schematic of a kit comprising a topical patch with an annulus of pressure-sensitive adhesive and a topical formulation in use and various features according to embodiments disclosed herein.

FIGS. 15A-D shows the process of using an topical patch according to embodiments disclosed herein with an annulus of pressure-sensitive adhesive and a topical formulation. FIG. 15A shows the topical patch comprising the annulus of pressure-sensitive adhesive and a backing film covering the interior open region of the annulus. FIG. 15B shows the application of the topical formulation onto the surface of the backing film. FIG. 15C shows application of the topical patch to a target dermal region of the subject, where the topical formulation is allowed to contact the skin at the target dermal region. The backing film may be used to hold the topical formulation in place and cause occlusion, enhancing the kinetics, penetration, and extent of delivery of the active agent into the target dermal region. The design on the backing film may be merely decorative and have no functional influence on the device once applied to a subject. FIG. 15D shows the removal of the backing film, exposing the affected skin following local anesthetization. FIG. 15E shows the injection with a syringe as an exemplary subsequent application, where local anesthetization with the topical formulation ameliorates or eliminates needle pain during injection.

### DETAILED DESCRIPTION

Disclosed herein are embodiments of formulations that are exceptionally effective at delivering local anesthetics such as lidocaine or other aminoamide or aminoester anesthetics, rapidly into the skin of a subject, such that local anesthesia is realized in less than, for example, one hour.

Aminoester local anesthetics include but are not limited to: procaine, benzocaine, chloroprocaine, cocaine, cyclomethycaine, dimethocaine (or larocaine), piperocaine, propoxycaine, procaine (or novocaine), proparacaine and tetracaine (or amethocaine). Aminoamide local anesthetics include but are not limited to: articaine, bupivacaine, cinchocaine (or dibucaine), etidocaine, levobupivacaine, lidocaine (or lignocaine), mepivacaine, prilocaine, ropivacaine and trimecaine.

Aminoester and aminoamide anesthetics can be administered topically to noninvasively block pain in humans and other animals. There is an ongoing need for a topical formulation that can deliver an amount of the local anesthetic to the epidermal and dermal tissues sufficient to provide anesthesia at an onset time of less than one hour, while otherwise being suitable for clinical use. Embodiments provided for herein satisfies these and other needs.

Owing to the huge benefit of using local anesthetics to treat pain in patients, numerous examples of topical and transdermal formulations of aminoester and aminoamide local anesthetics, alone and in various combinations, have been developed, for example: U.S. Pats. 5411738, 5827529, 6673363, 7883488, 9186334 and publications 2011/0288123, 2013/0184351, 2014/0141056, 2018/0326068, each of which is hereby expressly incorporated by reference in its entirety.

Several factors should be considered in assessing the utility of a local anesthetic product such as: (i) the potency of the local anesthetic and (ii) the mode of administration. One mode of administration for various dosage forms is topical, wherein the drug product is applied to the skin exterior and the active ingredient(s) diffuse from the formulation into the skin.

The skin, however, presents a formidable barrier to the delivery of drugs. Structurally, the skin consists of three principle parts: (i) a relatively thin outermost layer, the epidermis, (ii) a thicker inner region, the dermis, and (iii) the subcutaneous tissue, the lowermost layer also called the hypodermis or subcutis. The outermost layer of the epidermis, the stratum corneum, consists of corneocytes, flattened dead cells which are filled with keratin. The corneocytes are interconnected by corneodesmosomes and surrounded by lipids which form lamellar phases. The highly impermeable character of skin derives primarily from the stratum corneum. The viable epidermis underlying the stratum corneum is akin to other living tissue. The dermis provides the skin’s structural strength as well as the nerve and vascular networks that support the epidermis. In a topical mode of administration, the intended target tissue is the viable epidermis or, more typically, the dermis. This is in contrast to transdermal administration, in which the active ingredient permeates through the skin to be provided systemically via the vasculature or lymphatic system.

Delivering an active agent into (or through) the skin in sufficient concentration to provide therapeutic benefit usually requires some means for reducing the stratum corneum’s hindrance to ingress of the active agent. A number of physical methods for lowering the stratum corneum’s barrier properties have been developed, including electrically-assisted techniques such as iontophoresis or electroporation, ultrasound, heat, puncturing the stratum corneum with microneedle arrays, or ablation. Even for a single, non-repeated application, though, such physical methods have limitations, leading to very restricted use by patients in practice.

Even when physical methods are not deployed, it is well known that various factors can affect the permeation and absorption of an active agent from a skin-applied pharmaceutical preparation, including the nature of the active ingredient, the nature of the vehicle, the pH, and the relative solubility of the active molecule in the vehicle versus the skin. More specifically, active agent attributes such as molecular weight, lipophilicity or hydrophilicity, solubility, size and charge, melting point, as well as vehicle attributes such as active agent solubility and dissolution rate, ability to modulate the permeability of the stratum corneum, and physical characteristics such as occlusivity, spreadability and adhesion, can each have significant effects on permeability. Unfortunately, although skin permeability can be estimated for certain molecules under model conditions, it is generally not possible to predict such permeability from a practicable formulation.

Certain molecular agents, often termed penetration enhancers, chemical penetration enhancers, sorption promoters, sorption accelerants, or molecular penetration enhancers (“MPE™”s), can modulate skin permeability for a given molecule by, for example, disrupting the lipid bilayers of the stratum corneum. Over 600 substances have been identified as MPEs but few have been successfully developed into practical formulations. Many potent enhancers are irritating to the cells of the epidermis which limits both the choice and concentration of MPEs suitable for topical formulations. The MPEs of most practical utility are those that are known to cause at most limited irritation and sensitization of the skin, such as those provided in the ‘Inactive Ingredient Database’ (“IID”) published by the FDA. Notably, it is known in the art: (i) that the best skin permeation enhancement is typically found with combinations of MPEs, rather than by use of individual MPEs alone, (ii) that, in general, the penetration-enhancing roles of neither individual MPEs nor MPE combinations (typically termed ‘multiplexed MPE’s or “MMPE™”s) can be predicted, and (iii) that an MPE combination, or even an individual MPE, that performs well for one particular active compound will often perform substantially less well for a different active compound, especially one of a different molecular class.

Beyond the potency and extent of delivery of the active ingredient(s), other factors that need to be satisfied for a skin-applied drug formulation to be practicable include (i) chemical and physical stability, especially in the container-closure system in which the drug product will be distributed, stored and provided to subjects, (ii) what is termed the ‘esthetic profile’, the set of observations made by a subject when dispensing the product from the container-closure system and applying the product to the skin, including color, appearance, smell, sensation upon skin contact, ease and feel when spreading, rubbing and how these various characteristics develop with time (in the case of a patch or plaster product format, the equivalent attributes are more commonly termed ‘wear’ characteristics, including the ease of removal of the product from the packaging, application to the area of skin indicated by the package insert, the comfort during the period of application, whether there is discomfort or itch under the patch, the presence of ‘creep’ namely lateral reach of the adhesive leading to a dark tacky perimeter around the patch, and the ease and discomfort associated with removal at the end of the administration period), and (iii) the potential to irritate or sensitize the skin (and, if components in the product formulation absorb electromagnetic radiation in the visible or ultraviolet (“UV”) regions, the potential to irritate or sensitize the skin under photoirradiation).

Regulatory compliance is another important factor. It is the duty of a given regulatory agency, such as the United States Food and Drug Administration (“FDA”) to ensure that only pharmaceutical products that are safe and effective are made available to patients and consumers. A product might follow several routes to satisfy such FDA requirements. A product that adheres to the conditions stipulated in an FDA over the counter (“OTC”) monograph that provides acceptable ingredients, indication(s), doses, formulations, labeling and testing is generally regarded as safe and effective (“GRASE”) for the uses set forth in such monograph. A GRASE product may be marketed and sold in the US without the need for further regulatory filing. The OTC monograph for topical anesthetics lists benzocaine, cyclomethycaine sulfate, tetracaine, tetracaine hydrochloride, lidocaine, lidocaine hydrochloride, dibucaine and dibucaine hydrochloride as active ingredients that might be used prospectively in a monograph-compliant product. In the case of both lidocaine and lidocaine hydrochloride, monograph-compliant concentrations are in the range 0.4 to 4 % w/w.

The utility of a local anesthetic product is heavily dependent on another key parameter, namely the onset.

Amongst the many topical and transdermal formulations of local anesthetics taught in the prior art, few mention onset. It is, in fact, often accepted that the onset following topical administration of a lidocaine-comprising formulation is a characteristic of the lidocaine itself, and then largely independent of the details of the formulation from which it is provided.

When administered directly into a tissue provided with sensory nerves, lidocaine is known to have an onset of less than 2 min and a duration of 1 h to 2 h, although the pharmacokinetic (“PK”) and pharmacodynamic (“PD”) properties depend to some degree on the volume and concentration infused, the location of administration, and the tissue pH. The half-life of lidocaine elimination from the plasma following intravenous administration is approximately 1.8 h, with lidocaine and its metabolites being excreted by the kidneys.

In case of a formulation of lidocaine applied to the skin exterior, the availability of the lidocaine to nerve endings in the epidermis and dermis is subject to a lag time. The onset of action will always be longer than the lag time, as the concentration of active in the viable epidermis or dermis (or which is available to deeper tissue or systemically) must build from zero to a level at which a noticeable effect is evident.

Kreilgaard measured unbound cutaneous concentrations of lidocaine and prilocaine in rats by in vivo microdialysis following topical application of microemulsions, commercially available creams, and a hydrogel. Lidocaine lag times from each of the formulations (without recovery correction) are reported to be: Microemulsion A 21(±1) min, Microemulsion D 24(±7) min, Microemulsion G 15(±7) min, Xylocain® 20(±6) min, EMLA 38(±15) min were observed, where the compositions of microemulsions expressed as water:isostearylic isostearate: Labrasol/ Plurol Isostearique: lidocaine: prilocaine hydrochloride were A 20:10:70:23:5; D 7:70:23:17:0; E 11:26:63:0:2.4; and G 65:3:32:9.1/7.5:14.

Yamamoto et al. compared the permeation of five different drugs through cryopreserved excised human skin. They reported lag times for lidocaine of 0.5h (from ‘Penles tape 18 mg’) and of 1.4h (from ‘Emla cream’) (these values compare with lag times reported for Bisoprolol of 4.0h (from ‘Bisono tape 8 mg’), ; Nicotine of 0.7h (from ‘Nicotinell TTS20’); Rivastigmine of 1.6h (from ‘Rivastach patch 18 mg’), and Diclofenac 3.5h (from ‘Voltaren tape 30 mg’) and of 8.1h (from ‘Voltaren gel 1%’).

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, technical and scientific terms used herein have the same meaning as commonly understood by one of ordinary skill in the art to which the present disclosure belongs. For purposes of the present disclosure, the following terms are defined below.

### Definition of Terms

The terms “a”, “an”, or “the” as used herein not only include aspects with one member, but also include aspects with more than one member. For example, an embodiment including “a cellulosic thickening agent and a lower monohydric alcohol” should be understood to present certain aspects with at least a second cellulosic thickening agent, at least a second lower monohydric alcohol, or both. An embodiment including “an active agent” should be understood to present certain aspects with at least a second active agent, which may be of a different class (e.g., lidocaine with a non-steroidal anti-inflammatory drug, or with an anti-inflammatory steroid, or with a local anesthetic, or with a sunscreen agent).

The term “about” as used herein to modify a numerical value indicates a defined range around that value. If “X” were the value, “about X” would generally indicate a value from 0.95X to 1.05X. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X”. When the quantity “X” only includes whole-integer values (e.g., “X carbons”), “about X” indicates from (X-1) to (X+1). In this case, “about X” as used herein specifically indicates at least the values X, X-1, and X+1. When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 11%” is equivalent to “about 7%, about 9%, or about 11%.”

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein the term “Molecular Accelerant” or “MolAcc™” means an agent that reduces onset and/or lag time when incorporated within a topical formulation.

In compositions comprising an “additional” or “second” component, the second component, unless otherwise indicated, as used herein is chemically different from the other components or first component. A “third” component, unless otherwise indicated, is different from the other, first, and second components, and further enumerated or “additional” components are similarly different.

“Agent” as used herein indicates a compound or mixture of compounds that, when added to a composition, tend to produce a particular effect on the said composition’s properties or performance. For example, a composition comprising a thickening agent is likely to be more viscous than an otherwise identical comparative composition that lacks the thickening agent; a composition comprising an active agent is more likely to provide a beneficial effect in the subject to which the composition is administered that otherwise.

“Carbon atom number” or “C” is used in its conventional sense to mean the number of carbon atoms in an organic compound such as a fatty alcohol or fatty acid. Thus, stearyl alcohol (1-octadecanol), isostearyl alcohol (1-Heptadecanol, 16-methyl-), and oleyl alcohol (1-octadecenol) might each be referred to as C18 fatty alcohols; cetyl alcohol (1-hexadecanol) and palmitoleyl alcohol (cis-9-hexadecen-1-ol) might both be referred to as C16 fatty alcohols; lauric acid, myristic acid, palmitic acid and stearic acid might be referred to as a C12, a C14, a C16 and C18 fatty acid , respectively. In the conventional designation of fatty acids and fatty alcohols, the number of double bonds in the carbon chain is also provided. Thus oleic acid with a single carbon-carbon double bond is designated by (C18: 1), and arachidonic acid with four carbon-carbon double bonds is designated by (C20:4).

“C2-C4” as used herein refers to the group of organic compounds that have 2, 3, or 4 carbon atoms. “C2-C4 monohydric alcohol” as used herein refers to the group of monohydric alcohols that have 2, 3, or 4 carbon atoms, which includes but is not limited to ethanol, 1-propanol, 2-propanol (isopropanol), 1-butanol (n-butanol), 2-butanol (sec-butanol), 2-methylpropan-1-ol (isobutanol), or 2-methylpropanol (tert-butanol), or any combination or mixture thereof. “C14-C18” as used herein refers to the group of organic compounds that have 14, 15, 16, 17, or 18 carbon atoms. “C14-C18 fatty acid” as used herein refers to the group of fatty acids that have 14, 15, 16, 17, or 18 carbon atoms, which includes but is not limited to myristic acid, pentadecanoic (pentadecylic) acid, palmitic acid, margaric acid, stearic acid, isostearic acid, myristoleic acid, myristovaccenic acid, myristolinolenic acid, 8-tetradecenoic acid, pentadecanoic acid, palmitolinolenic acid, palmitidonic acid, palmitovaccenic acid, palmitoleic acid, sapienic acid, 4-hexadecanoic acid, α-linolenic acid, stearidonic acid, α-eleostearic acid, β-eleostearic acid, punicic acid, 7,10,13-ocadecatrienoic acid, 12-octadecenoic acid, linoleic acid, linolelaidic acid, γ-linolenic acid, calendic acid, pinolenic acid, vaccenic acid, rumenic acid, oleic acid, elaidic acid, or petroselenic acid, or any combination or mixture thereof. “C14-C20” as used herein refers to the group of organic compounds that have 14, 15, 16, 17, 18, 19, or 20 carbon atoms. “Monohydric C14-C20 alcohol” as used herein refers to the group of monohydric alcohols that have 14, 15, 16, 17, 18, 19, or 20 carbon atoms and includes but is not limited to myristyl alcohol, pentadecyl alcohol, palmityl alcohol (cetyl alcohol), margaryl alcohol, stearyl alcohol, isostearyl alcohol, nonadecyl alcohol, arachidyl alcohol, myristoleyl alcohol, myristovaccenyl alcohol, myristolinolenyl alcohol, 8-tetradecenyl alcohol, pentadecanyl alcohol, palmitolinolenyl alcohol, palmitidonyl alcohol, palmitovaccenyl alcohol, palmitoleyl alcohol, sapienyl alcohol, 4-hexadecanyl alcohol, α-linolenyl alcohol, stearidonyl alcohol, α-eleostearyl alcohol, β-eleostearyl alcohol, punicyl alcohol, 7,10,13-ocadecatrienyl alcohol, 12-octadecenyl alcohol, linoleyl alcohol, linolelaidyl alcohol, γ-linolenyl alcohol, calendyl alcohol, pinolenyl alcohol, vaccenyl alcohol, rumenyl alcohol, oleyl alcohol, elaidyl alcohol, petroselenyl alcohol, 1-tetradecanol, cetostearyl alcohol, dihomo-α-linolenyl alcohol, eicosatetraenyl alcohol, eicosapentaenyl alcohol, 9,12,15-eicosatrienyl alcohol, β-eicosatetraenyl alcohol, dihomo-linoleyl alcohol, dihomo-γ-linoleyl alcohol, arachidonyl alcohol, paullinyl alcohol, 7,10,13-eicosatrienyl alcohol, gondoyl (11-eicosenoyl) alcohol, 8-11-eicosadienyl alcohol, gadoleyl alcohol, 8-eicosenyl alcohol, or any combination or mixture thereof. In general, the term “C(A)-C(B)” as used herein refers to the group of organic compounds that have (A) carbon atoms, (B) carbon atoms, or between (A) and (B) carbon atoms, or any combination or mixture thereof.

“Cellulosic thickening agent” as used herein includes a thickening agent that is a natural or synthetic oligomeric or polymeric carbohydrate (e.g., cellulose and pharmaceutically acceptable vegetable gums) or a polymeric or oligomeric derivative of a polymeric carbohydrate that is produced by chemical modification (e.g., hydroxypropyl cellulose, hydroxypropyl methyl cellulose, hydroxyethyl cellulose). Representative cellulosic thickening agents include cellulose, hydroxypropyl cellulose (“HPC”), hydroxypropyl methyl cellulose (“HPMC”), hydroxyethyl cellulose (“HEC”), methyl cellulose, carboxymethyl cellulose, and the like.

The terms “chassis”, “vehicle” and “base formulation” as used interchangeably herein are equivalent terms that include a plurality of solvents or other excipients that comprise the bulk of a formulation, into which one or more active agents or additional agents might be introduced.

As used herein the term “comparative formulation” as it relates to a first formulation containing an active ingredient refers to a second formulation containing the same active ingredient. Generally, for the second formulation to qualify as a comparative formulation the concentration of the active ingredients should be approximately the same in the first and second formulation.

As used herein, the phrase “effective amount” or “effective dose” means an amount sufficient to achieve the desired result and accordingly will depend on the ingredient and its desired result. Nonetheless, once the desired effect is known, determining the effective amount is within the skill of a person skilled in the art.

Unless otherwise indicated, the “error bars” provided in the figures represent the standard error of the mean value, whereas the top of the solid, shaded bar represents a single data value, which is the mean value of the distribution of data values.

“Finite dosing” as used herein generally includes an application of a limited formulation dose such as to provide a limited reservoir of an active agent, other agents and chassis. The active agent or one or more other agents in the reservoir is depleted with time, leading to a decrease of the delivery rate after a maximum rate has been reached and perhaps maintained for a period. Similarly “infinite dosing” as used herein generally includes an application of a substantial formulation dose such as to provide an effectively non-limited reservoir of an active agent, or one or more other agents. The agent(s) in the reservoir is little depleted with time, potentially allowing a delivery rate to be maintained for a longer period.

“Flux” as used herein refers to the amount of a substance delivered into or through a unit area of a membrane in unit time. Flux measurements may be made in vitro using Franz diffusion cells. Suitable membranes for flux studies include synthetic membranes and mammalian skin including human cadaver skin and porcine skin. Flux measurements may also be made in vivo using pharmacokinetic studies and the like.

“Formulation,” “pharmaceutical composition,” and “composition” as used interchangeably herein are equivalent terms referring to a composition of matter for administration to a subject.

“Gel” as used herein means a semisolid system consisting of either a suspension made up of small inorganic particles or large organic molecules interpenetrated by a liquid. The viscosity of a gel may be such that it is either flowable or non-flowable.

“Irritancy Score” as used herein means a subjective rating of the extent of skin irritation caused by a test composition after such composition is applied to the human forearm. The value of the Irritancy Score ranges from 1 (no detectable irritation) to 10 (severe irritation prompting premature removal of the test composition).

The term “lagtime” or “lag time” as used herein means the x-axis intercept that results from extrapolating the steady-state line of a plot of the cumulative amount of active that has permeated through the membrane or membrane section (y-axis) against time (x-axis). For skin as the membrane, lag time may, as stipulated by the context, refer to permeation through the stratum corneum, through the stratum corneum and epidermis, or through the stratum corneum, epidermis and dermis.

“Lower alcohol” as used herein includes straight- or branched-chain alkyl alcohols with one or more hydroxyl groups that comprise less than seven (7) carbon atoms. Representative lower alcohols include methanol, ethanol, n-propanol, isopropanol, n-butanol, t-butanol, n-pentanol, 3-pentanol, n-hexanol, 2-methoxyethanol, 2-(2-ethoxyethoxy)ethanol, propylene glycol (propane-1,2-diol), butanediol, butynediol, pentanediol, hexanediol, hexane triol and the like.

The prefix “micro” as used herein can be alternatively abbreviated as “µ” or “u.” For example, micrograms are typically abbreviated as µ g, but can alternatively be abbreviated as “ug” .

“Monohydric alcohol” as used herein includes straight- or branched-chain alkyl alcohols with a single hydroxyl group. Representative monohydric alcohols include methanol, ethanol, n-propanol, isopropanol, n-butanol, t-butanol, n-pentanol, 3-pentanol, n-hexanol, 2-methoxyethanol, 2-(2-ethoxyethoxy)ethanol, hexadecan-1-ol, oleyl alcohol, isostearyl alcohol and the like.

As used herein the term “multiplexed molecular penetration enhancer” or “MMPE” means a penetration enhancer system comprising two or more substances wherein each of the two or more substances is also a molecular penetration enhancer.

The term “onset of action” as used herein means the amount of time it takes for the effects of an active to become noticeable after administration in a formulation to the skin.

The term “or” as used herein should in general be construed non-exclusively. For example, an embodiment of “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

The term “organic solvent” as used herein refers to a pure substance or a mixture of substances that is (a) a liquid at an operating temperature such as room temperature, (b) contains at least one carbon atom, and optionally at least one hydrogen atom, and (c) is capable of dissolving another substance to create a solution. Organic solvents have different volatilities. Dimethyl sulfoxide (“DMSO”), for example, has a lower volatility than acetone.

“Pain Score” as used herein means a subjective rating of effectiveness of a test composition at inducing local anesthesia in a subject 45 minutes after such composition is applied to the human forearm. The value of the Pain Score ranges from 1 (no detectable numbness or anesthesia) to 10 (complete local anesthesia and numbness).

“Penetration enhancer”, “chemical penetration enhancer”, “molecular penetration enhancer” or “MPE” as used interchangeably herein includes an agent or a combination of agents that improves the transport of molecules such as a pharmaceutically or cosmetically active agent into or through a natural membrane such as skin or nail. A molecular penetration enhancer may be used to assist in the delivery of an active agent topically, regionally, or transdermally. A molecular penetration enhancer may be a pure substance or may comprise a mixture of different chemical entities. Examples of substances that may act as MPEs include, but are not limited to: (+/-)-limonene; lauric acid; glycerol; isopropyl alcohol; isopropyl myristate; oleic acid; propylene glycol; Transcutol® (di(ethylene glycol) ethyl ether); and azone (1-dodecylazacycloheptan-2-one).

The term “pH adjusting agent” as used herein refers to an agent added to the compositions of the present application for the purpose of changing the pH of the composition. Examples of such agents include acids, bases, and buffers that are each pharmaceutically acceptable or cosmetically acceptable.

The term “pharmaceutically acceptable” means compatible with the treatment of animals, and in particular, humans.

The term “pharmaceutically acceptable salt” means a pharmaceutically acceptable acid addition salt or a pharmaceutically acceptable base addition salt. The formation of a desired compound salt is achieved using standard techniques. For example, the neutral compound is treated with an acid or base in a suitable solvent and the formed salt is isolated by filtration, extraction, or by any other suitable method.

The term “potency” is interpreted herein to mean the specific ability or capacity of the product, as indicated by appropriate laboratory tests or by adequately controlled clinical data obtained through the administration of the product in the manner intended, to effect a given result.

“Regional delivery” as used herein means delivery of an agent through the skin but concentrating in proximate tissue or joint.

“Saturation concentration” of a solute as used herein means the concentration of a solution at which no more the solute will dissolve in the solution.

“Strength” as used herein when applied to a formulation means the amount of a substance per unit amount of the formulation. For semisolid dosage forms and solutions, the strength of a particular substance can be conveniently characterized as the concentration of the substance usually expressed as a percentage of weight by weight.

“Solubilizing agent” as used herein means an agent that is added to a solvent system to enhance the solubility of a given active agent in the resulting medium. Examples of solubilizing agents include, but are not limited to, 2-hydroxypropyl-β-cyclodextrin; sorbitan monolaurate; sulfobutylether-β-cyclodextrin (Captisol); Transcutol P and Tween 80.

The term “subject” as used herein includes all members of the animal kingdom, preferably mammals, and most preferably, humans.

“Superficial delivery” as used herein means delivery of an agent to the skin exterior surface only.

“Surfactant” as used herein includes a surface-active agent. Surfactants reduce the surface tension of a solvent in which they are dissolved.

“Thickening agent” as used herein includes an agent or combination of agents that increases the viscosity of a composition. A thickening agent may be a pure substance, or it may comprise, consist essentially of, or consist of a mixture of different chemical entities. Exemplary thickening agents include cellulosic thickening agents, other polysaccharides such as chitosan and the like, carbomer polymers, carbomer derivatives, polyvinyl alcohol, poloxamers, natural gums, as well as mixtures thereof.

“Topical delivery” is used in its conventional sense to mean delivery of an agent, such as a therapeutically active agent, into the viable skin. The outermost layer of the epidermis, the stratum corneum, is lifeless and topical delivery then refers to delivery into the viable epidermis and/or dermis. Topical delivery of a drug may be the advantageous basis, for example, for treatment of various skin disorders. Topical delivery also refers to delivery of an agent into other tissues that are exposed to the environment exterior to the body, such as nail, mucosa or eye.

“Topical formulation” as used herein includes, in one aspect, a composition that is suitable for topical application to the skin, nail, or mucosa. A topical formulation may, for example, be used to confer a therapeutic or cosmetic benefit to its user. Specific topical formulations can be used for superficial, local, regional, or transdermal delivery of substances. The term “topical formulation” as used herein also encompasses the compositions that are formed once a composition that is suitable for topical application to the skin, nail or mucosa is applied to the skin, nail or mucosa. The composition of the topical formulation resulting from such application to the skin, nail or mucosa may differ from the originally applied formulation. For example, components from the originally applied formulation may undergo differential evaporation causing the relative amounts of the ingredients in the formulation to change. Additionally, components from the originally applied formulation may diffuse into the skin, nail or mucosa. Further, compounds that are on or within the substrate to which the formulation is applied may become incorporated into the formulation. For example, linolenic acid and cholesterol, which are natural components of skin, may become incorporated into the formulation.

The term “topical administration” or equivalently “topical application” is used in its conventional sense to mean application of a substance, such as a formulation containing an active agent, to the skin or to a localized more or less external region of the body such as the nail, mucosa or eye. Topical administration may result in any one or more of superficial, topical, regional or transdermal delivery the active agent.

“Transdermal” as used herein includes a process that occurs through the skin. The terms “transdermal,” “percutaneous,” and “transcutaneous” can be used interchangeably. In certain embodiments, “transdermal” may also include epicutaneous.

“Transdermal delivery” is used in its conventional sense to mean provision of an agent transdermally, that is through the skin, for systemic availability. Following passage through the skin the agent is made available to tissues throughout the body via the lymphatic system and/or the vasculature.

The term “treating” or “treatment” as used herein (and as well understood in the art) means an approach for obtaining beneficial or desired results in a subject’s condition, including clinical results. Beneficial or desired clinical results can include, but are not limited to, alleviation or amelioration of one or more symptoms or conditions, diminishment of the extent of a disease, stabilizing (e.g., not worsening) the state of disease, prevention of a disease’s transmission or spread, delaying or slowing of disease progression, amelioration or palliation of the disease state, diminishment of the reoccurrence of disease, and remission, whether partial or total and whether detectable or undetectable. “Treating” and “treatment” as used herein also include prophylactic treatment. Treatment methods comprise administering to a subject a therapeutically effective amount of an active agent. The administering step may consist of a single administration or may comprise a series of administrations. The compositions are administered to the subject in an amount and for a duration sufficient to treat the patient. The length of the treatment period depends on a variety of factors, such as the severity of the condition, the age and genetic profile of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required.

The term “volatility” as used herein refers to the rate at which a substance evaporates. A more volatile organic solvent evaporates more rapidly than a less volatile solvent when each is held at room temperature and pressure.

The term “solubility” as used herein has its ordinary meaning as understood in light of the specification and refers to the ability or extent to which a compound (solute) can dissolve in a solvent. The United States Pharmacopeia and British Pharmacopeia categorize solubility as follows: very soluble, less than 1 parts solvent per 1 part solute; freely soluble, from 1 to 30 parts solvent per 1 part solute; soluble, from 10 to 30 parts solvent per 1 part solute; sparingly soluble, from 30 to 100 parts solvent per 1 part solute; slightly soluble, from 100 to 1000 parts solvent per 1 part solute; very slightly soluble, from 1000 to 10000 parts solvent to 1 part solute; practically insoluble or insoluble, 10000 or more parts solvent per 1 part solute. The solubility properties of an active pharmaceutical ingredient affect its pharmacokinetics and pharmacodynamics, e.g., dissolution rate, transdermal absorption, absorption in the digestive tract, metabolism, excretion and clearance, permeability throughout the body, transit across the blood-brain barrier. In some embodiments, solubility of the local anesthetics and formulations comprising a local anesthetic describe herein is improved with the addition of excipients including but not limited to monohydric alcohols, terpenes, terpenoids, isopropyl esters of fatty acids, nonionic surfactants, MolAccs, MPEs, emollients, solubilizing agents, antioxidants, preservatives, chelating agents, organic solvents, or any combination thereof.

As used herein, the term “bioavailability” has its ordinary meaning as understood in light of the specification and refers to the measure of the percentage of active pharmaceutical ingredient that reaches systemic circulation compared to the total dose amount administered, which can be determined by measuring drug concentration in plasma quantitatively. Intravenous administration results in a bioavailability of 100%. Bioavailability of other administration methods, e.g. topical, are dictated by factors including but not limited to solubility, permeability, metabolism, degradation, excretion and clearance, or modified release formulations. High bioavailability of topical formulations may be a good indicator for potential toxicity and unwanted adverse effects. In some embodiments, bioavailability of the local anesthetics and formulations comprising a local anesthetic described herein may be modulated with the addition of excipients including but not limited to monohydric alcohols, terpenes, terpenoids, isopropyl esters of fatty acids, nonionic surfactants, MolAccs, MPEs, emollients, solubilizing agents, antioxidants, preservatives, chelating agents, organic solvents, or any combination thereof.

As used herein, the terms “efficacy”, “intrinsic activity”, and “potency” have their ordinary meaning as understood in light of the specification and refers to the amount of active pharmaceutical ingredient that is needed to achieve a desired effect. In some embodiments, the desired effect is sufficient local loss of sensation or pain in a patient and can be quantified with a Pain Score. Efficacy or potency can be quantified as the median or 50% effective dose (ED₅₀), the dose that imparts a measurable effect in 50% of a population, or the 95% effective dose, the dose that imparts a measurable effect in 95% of a population. In some embodiments, efficacy or potency of the local anesthetics and formulations comprising a local anesthetic described herein may be improved with the addition of excipients including but not limited to monohydric alcohols, terpenes, terpenoids, isopropyl esters of fatty acids, nonionic surfactants, MolAccs, MPEs, emollients, solubilizing agents, antioxidants, preservatives, chelating agents, organic solvents, or any combination thereof.

As used herein, the term “tolerability” has its ordinary meaning as understood in light of the specification and refers to the extent to which a patient will willingly withstand adverse or side effects of an active compound to achieve the desired therapeutic effect, and can refer to short-term or long-term side effects. In some embodiments, tolerability of the local anesthetics and formulations comprising a local anesthetic described herein may be improved with the addition of excipients including but not limited to monohydric alcohols, terpenes, terpenoids, isopropyl esters of fatty acids, nonionic surfactants, MolAccs, MPEs, emollients, solubilizing agents, antioxidants, preservatives, chelating agents, organic solvents, or any combination thereof.

As used herein, the term “stability” has its ordinary meaning as understood in light of the specification and refers to the ability for the active compound or the formulation containing the active compound to remain effective, intact, and safe for consumption or administration over time under the influence of environmental factors such as temperature, humidity, and light. Stability can be assessed by forced degradation studies according to parameters, conditions, and standards set forth by the Food and Drug Administration (FDA) and International Council for Harmonisation (ICH). In some embodiments, stability of the local anesthetics and formulations comprising a local anesthetic described herein may be improved with the addition of excipients including but not limited to monohydric alcohols, terpenes, terpenoids, isopropyl esters of fatty acids, nonionic surfactants, MolAccs, MPEs, emollients, solubilizing agents, antioxidants, preservatives, chelating agents, organic solvents, or any combination thereof.

As used herein, the term “dosage forms” has its ordinary meaning as understood in light of the specification and refers to the methods of delivering the active pharmaceutical ingredient and any excipients to an individual. Methods of delivery include but are not limited to oral, pills, tablets, capsules, films, drinks, syrups, powders, pastes, inhalation, aerosol, smoke, vapor, mist, buccal, sublingual, nasal, suppository, parenteral, intradermal, subcutaneous, intramuscular, intraosseous, intraperitoneal, intravenous, topical, cream, gel, liniment, balm, lotion, ointment, liquid drops, or patches. Certain methods of delivery may be preferential to others, for reasons such as comfort to the patient, invasiveness, side effects, level of degradation, e.g. in the digestive tract, level of absorption, onset of action, local vs. general effect, duration of effect, effective dose amount, or therapeutic index. In some embodiments, dosage forms of the local anesthetics and formulations comprising a local anesthetic described herein may be improved with the addition of excipients including but not limited to monohydric alcohols, terpenes, terpenoids, isopropyl esters of fatty acids, nonionic surfactants, MolAccs, MPEs, emollients, solubilizing agents, antioxidants, preservatives, chelating agents, organic solvents, or any combination thereof.

The term “% w/w” or “% wt/wt” means a percentage expressed in terms of the weight of the ingredient or agent over the total weight of the composition multiplied by 100.

### Compositions

The present application includes compositions comprising one or more active ingredients for topical administration and for topical, regional or transdermal delivery.

In an embodiment, the composition is a pharmaceutical composition for human or veterinary use.

In another embodiments the composition comprises a local anesthetic drug. Active Agent

The present disclosure provides for compositions and formulations comprising at least one active agent.

In one embodiment, the at least one active agent is a pharmaceutical agent, a cosmeceutical agent, a cosmetic agent, a nutritional supplement, or a diagnostic agent.

Non-limiting examples of active agents include aminoamide and aminoester local anesthetics.

In some embodiments, the active agent is an aminoamide or aminoester local anesthetic.

In one embodiment, the active agent is lidocaine.

In additional embodiments, the compositions of the formulation comprise two or more active agents. More preferably one of the two or more active agents comprises an aminoamide or aminoester local anesthetics. Still more preferably one of the two or more active agents comprises lidocaine.

In additional embodiments, the compositions of the formulation comprise lidocaine and a second aminoamide or aminoester local anesthetic. Alternatively, the second aminoamide or aminoester local anesthetic comprises prilocaine or tetracaine.

In embodiments provided for in the present disclosure, the active agent may be present in an amount sufficient to provide a physiological, health, skin care and/or cosmetic benefit. In some embodiments, the active agent is present in an amount of 0.0001, 0.001, 0.01, 0.1, 1, 5, 10, 12, 25, 30, 40, or 50 % w/w or any value within the range defined by any two of the aforementioned values. In some embodiments, the active agent is present in an amount of at least 0.001% w/w. In some embodiments, the active agent is present in an amount of at least 0.01% w/w or at least 0.1% w/w. In some embodiments, the active agent is present in an amount of at least 1% w/w. In some embodiments, the active agent is present in an amount of about 1% w/w to about 12% w/w, including any amount between those listed. In some embodiments, the active agent is present in an amount of about 5% w/w to about 25% w/w, including any amount between those listed. In a particular embodiment of the disclosure, the active agent is used at a concentration of about 1% to about 20% w/w, including any amount between those listed.

In several embodiments the compositions of the formulation comprise an aminoamide or aminoester local anesthetic that is provided in the FDA monograph referring to ‘External Analgesic Drug Products For Over-The Counter Human Use’ and the concentration of said aminoamide or aminoester local anesthetic is within the composition range provide for such agent in said monograph.

In several embodiments, the compositions of the formulation comprise lidocaine in an amount of between 0.5 and 5.0% w/w, including for example, 0.5%, 1%, 1.5%, 2%, 2.5%, 3%, 3.5%, 4%, 4.5%, 5%, about 0.5%, about 1%, about 1.5%, about 2%, about 2.5%, about 3%, about 3.5%, about 4%, about 4.5%, or about 5%, including any amount between those listed.

An embodiment including “an active agent” should be understood to present certain aspects with at least a second active agent, which may be the same class or a different class (for example, lidocaine with an anti-inflammatory agent, or with a cannabinoid such as cannabidiol).

### Monohydric Alcohol

In several embodiments, the compositions and formulations include at least one monohydric alcohol. Suitable monohydric alcohols include, but are not limited to, ethanol, propanol, propan-2-ol, (isopropanol), butanol, butan-2-ol (isobutanol), pentanol, pentan-2-ol, pentan-3-ol, 3-methyl-2-butanol, hexanol, hexan-2-ol, hexan-3-ol, benzyl alcohol, stearyl alcohol (1-octadecanol), isostearyl alcohol (1-Heptadecanol, 16-methyl-), oleyl alcohol (1-octadecenol), cetyl alcohol (1-hexadecanol), palmitoleyl alcohol and the like, as well as a mixture thereof.

In some embodiments, the formulations include at least one lower monohydric alcohol. Example lower monohydric alcohols include, but are not limited to, ethanol, propanol, propan-2-ol, (isopropanol), butanol, butan-2-ol (isobutanol), pentanol, pentan-2-ol, pentan-3-ol, 3-methyl-2-butanol, hexanol, hexan-2-ol, hexan-3-ol, benzyl alcohol and the like, as well as a mixture thereof.

In some embodiments, the monohydric alcohol is ethanol.

In several such embodiments, the ethanol is present in an amount of between 0% and about 90% w/w or between 0% and about 90%. In some embodiments, ethanol is present in an amount of about 0%, 10%, 20%, 30%, 31%, 32%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 64%, 69%, 72%, 80%, 86%, 90% w/w, or about 0%, about 10%, about 20%, about 30%, about 31%, about 32%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 64%, about 69%, about 72%, about 80%, about 86%, or about 90% w/w, or any amount between a range defined by any two aforementioned numbers.

In some embodiments, the monohydric alcohol is benzyl alcohol.

In several embodiments, the benzyl alcohol is present in an amount between 0% and 20% w/w. In certain such embodiments, benzyl alcohol is present in an amount of 0%, 5%, 10%, 15%, or 20% w/w, or about 0%, about 5%, about 10%, about 15%, or about 20% w/w or any amount within a range defined by any two of the aforementioned numbers.

In some embodiments, the formulations include a fatty alcohol.

Example fatty alcohols include but are not limited to stearyl alcohol (1-octadecanol), isostearyl alcohol (1-Heptadecanol, 16-methyl-), oleyl alcohol (1-octadecenol), cetyl alcohol (1-hexadecanol), palmitoleyl alcohol and the like, as well as a mixture thereof.

In several embodiments, the fatty alcohol is a C14, a C16, a C18 or a C20 fatty alcohol.

In some embodiments, the fatty alcohol is a C18 fatty alcohol.

In some embodiments, the fatty alcohol is isostearyl alcohol.

In some embodiments, the isostearyl alcohol is present in an amount of between 0% and 30% w/w. In certain such embodiments, isostearyl alcohol is present in an amount of 0%, 1%, 2%, 3%, 4%, 5%, 6%, 7%, 8%, 9%, 10%, 15%, 20%, 21%, 25%, or 30% w/w, or about 0%, about 1%, about 2%, about 3%, about 4%, about 5%, about 6%, about 7%, about 8%, about 9%, about 10%, about 15%, about 20%, about 21%, about 25%, or about 30% w/w or any amount within a range defined by any two of the aforementioned numbers.

### Combination of Monohydric Alcohols

In some embodiments, the compositions and formulations include more than one monohydric alcohol.

In additional embodiments, the compositions and formulations include a lower monohydric alcohol and a fatty alcohol.

In several embodiments, the formulations include a lower monohydric alcohol including but not limited to ethanol, propanol, propan-2-ol, (isopropanol), butanol, butan-2-ol (isobutanol), pentanol, pentan-2-ol, pentan-3-ol, 3-methyl-2-butanol, hexanol, hexan-2-ol, hexan-3-ol, benzyl alcohol, or any combination or mixture thereof, as well as a combination or mixture thereof with a fatty alcohol.

In some embodiments, the monohydric alcohol included in combination with a fatty alcohol is ethanol.

In some embodiments, the monohydric alcohol included in combination with a fatty alcohol is benzyl alcohol.

In some embodiments, the fatty alcohol combined with a lower monohydric alcohol is a C14, a C16, a C18 or a C20 fatty alcohol.

In some embodiments, the fatty alcohol combined with a lower monohydric alcohol is a C18 fatty alcohol.

In some embodiments, the fatty alcohol combined with a lower monohydric alcohol is isostearyl alcohol.

In some embodiments, the compositions and formulations include ethanol and isostearyl alcohol.

In some embodiments, the ethanol is present in an amount between 0% and 90% w/w or between about 0% and about 90%. In some embodiments, ethanol is present in an amount of 0%, 10%, 20%, 30%, 31%, 32%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 64%, 69%, 72%, 80%, 86%, 90% w/w, or about 0%, about 10%, about 20%, about 30%, about 31%, about 32%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 64%, about 69%, about 72%, about 80%, about 86%, or about 90% w/w, or any amount between a range defined by any two aforementioned numbers.

In some embodiments, the isostearyl alcohol is present in an amount between 0% and 30% w/w or about 0% and about 30% w/w. In certain such embodiments, isostearyl alcohol is present in an amount of 0%, 1%, 2%, 3%, 4%, 5%, 6%, 7%, 8%, 9%, 10%, 15%, 20%, 21%, 25%, or 30% w/w, or about 0%, about 1%, about 2%, about 3%, about 4%, about 5%, about 6%, about 7%, about 8%, about 9%, about 10%, about 15%, about 20%, about 21%, about 25%, or about 30% w/w or any amount within a range defined by any two of the aforementioned numbers.

### Terpene and Terpenoid

In some embodiments, the compositions and formulations include a light terpene or terpenoid. Terpenes are organic compounds produced by plants or some insects and animals and include but are not limited to light terpenes, hemiterpenes, monoterpenes, sesquiterpenes, diterpenes, sesterterpenes, triterpenes, sesquarterpenes, tetraterpenes, polyterpenes, norisoprenoids, isoprene, prenol, isovaleric, geraniol, terpineol, limonene, myrcene, linalool, pinene, iridoids, humulene, farnesenes, farnesol, cafesol, kahweol, cembrene, taxadiene or squalene, or any combination or mixture thereof. Terpenoids, or isoprenoids, are molecules or compounds derived from terpenes, and include but are not limited to hemiterpenoids, monoterpenoids, sesquiterpenoids, diterpenoids, sesterterpenoids, triterpenoids, tetraterpenoids, polyterpenoids, citral, menthol, camphor, cannabinoids, ginkgolide, bilobalide, cucurminoids, retinol, retinal, phytol, lanosterol, cycloartenol, steroids, lycopene, α-carotene, β-carotene, or γ-carotene, or any combination or mixture thereof.

In some embodiments, suitable light terpenes and terpenoids include geraniol, terpineol, limonene, myrcene, linalool and pinene.

In some embodiments, the light terpene is limonene.

In some embodiments, the limonene is present in an amount between 0% and 60% w/w, or between about 0% and 60%. In some embodiments, the limonene is present in an amount of 0%, 1%, 5%, 10%, 15%, 20%, 25%, 30%, 35%, 40%, 45%, 50%, 55%, 60% w/w or about 0%, about 1%, about 5%, about 10%, about 15%, about 20%, about 25%, about 30%, about 35%, about 40%, about 45%, about 50%, about 55%, about 60% w/w or any amount within a range defined by any two of the aforementioned numbers.

### Isopropyl Ester of a Fatty Acid

In some embodiments, the composition includes an ester of a fatty acid.

In some embodiments, the composition includes an isopropyl ester of a fatty acid selected lauric acid (C12:0), tridecylic acid (C13:0), myristic acid (C14:0), pentadecylic acid (C15:0), palmitic acid (C16:0), margaric acid (C17:0), stearic acid (C18:0), nonadecylic acid (C19:0), arachidic acid (C20:0), α-linolenic acid (C18:3), stearidonic acid (C18:4), linoleic acid (C18:2), linolelaidic acid (C18:2), γ-linolenic acid (C18:3), palmitoleic acid (C16:1), vaccenic acid (C18:1), oleic acid (C18:1) and elaidic acid (C18:1).

In some embodiments, compositions include isopropyl myristate, isopropyl palmitate, diisopropyl adipate, glycerol monooleate, or diethyl sebacate.

In some embodiments, the ester of a fatty acid is present in an amount between 0% and 30% w/w or between about 0% and about 30% w/w. In some embodiments, the ester of a fatty acid is present in an amount of 0%, 1%, 2%, 3%, 4%, 5%, 6%, 7%, 8%, 9%, 10%, 11%, 12%, 13%, 14%, 15%, 16%, 17%, 18%, 19%, 20%, 21%, 25%, 26%, or 30% w/w, or about 0%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 25%, about 26%, or about 30% w/w, or an amount within a range defined by any two of the aforementioned numbers.

### Combination of Fatty Acid Isopropyl Esters

In some embodiments, the compositions and formulations include at least one ester of a fatty acid.

In some embodiments, the composition includes the esters of at least one fatty acid selected from lauric acid (C12:0), tridecylic acid (C13:0), myristic acid (C14:0), pentadecylic acid (C15:0), palmitic acid (C16:0), margaric acid (C17:0), stearic acid (C18:0), nonadecylic acid (C19:0), arachidic acid (C20:0), α-linolenic acid (C18:3), stearidonic acid (C18:4),, linoleic acid (C18:2), linolelaidic acid (C18:2), γ-linolenic acid (C18:3), palmitoleic acid (C16:1), vaccenic acid (C18:1), oleic acid (C18:1) and elaidic acid (C18:1)..

In some embodiments, the composition includes the isopropyl esters of two fatty acids.

In some embodiments, the composition includes isopropyl myristate and isopropyl palmitate.

In some embodiments, the isopropyl myristate is present in an amount greater than that of the isopropyl palmitate, or the isopropyl myristate is present in an amount lesser than that of the isopropyl palmitate.

In some embodiments, the isopropyl myristate and isopropyl palmitate are each present in an amount between 0% and 30% w/w or between about 0% and about 30% w/w. In some embodiments, the isopropyl myristate or isopropyl palmitate are each present in an amount of 0%, 1%, 2%, 3%, 4%, 5%, 6%, 7%, 8%, 9%, 10%, 11%, 12%, 13%, 14%, 15%, 16%, 17%, 18%, 19%, 20%, 21%, 25%, 26%, or 30% w/w, or about 0%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 25%, about 26%, or about 30% w/w, or an amount within a range defined by any two of the aforementioned numbers.

### Nonionic Surfactant

In some embodiments, the composition includes at least one pharmaceutically acceptable surfactant that is a nonionic surfactant.

In the present invention the nonionic surfactant may be one or more of 2,4,7,9-tetramethyl-5-decyne-4,7-diol ethoxylate average Mn 670; 2,4,7,9-tetramethyl-5-decyne-4,7-diol, mixture of (±) and meso 98%; Adogen® 464; ALKANOL® 6112; alkyl polyglycoside; anhydrosorbitol ester; Brij® 58; Brij® 93; Brij® C10; Brij® L4 (polyethylene glycol dodecyl ether); Brij® O10; Brij® O20; Brij® S100; Brij® S10; Brij® S20; carboxylic amides; carboxylic esters; Cetomacrogol 1000; cetostearyl alcohol; cetyl alcohol; Cocamide diethanolamine (“DEA”); Cocamide monoethanolamine (“MEA”); decyl glucoside; decyl polyglucose; disodium cocoamphodiacetate; ethoxylated aliphatic alcohol; ethoxylated derivatives of anhydrosorbitol ester; ethylenediamine tetrakis(ethoxylate-block-propoxylate) tetrol average Mn ~7,200; ethylenediamine tetrakis(ethoxylate-block-propoxylate) tetrol average Mn ~8,000; ethylenediamine tetrakis(propoxylate-block-ethoxylate) tetrol average Mn ~3,600; glycerol monostearate; glycol esters of fatty acids; IGEPAL CA-630; IGEPAL® CA-520; IGEPAL® CA-720; IGEPAL® CO-520; IGEPAL® CO-630; IGEPAL® CO-720; IGEPAL® CO-890; IGEPAL® DM-970; isoceteth-20; lauryl glucoside; maltosides; MERPOL® A; MERPOL® DA; MERPOL® HCS; MERPOL® OJ; MERPOL® SE; MERPOL® SH; monoalkanolamine condensates; monolaurin; mycosubtilin; narrow-range ethoxylate; N-octyl beta-D-thioglucopyranoside; Nonidet P-40; Nonoxynol-9; Nonoxynols; NP-40; octaethylene glycol monododecyl ether; octyl glucoside; oleyl alcohol; polyethylene glycol (“PEG”)-10 sunflower glycerides; pentaethylene glycol monododecyl ether; polidocanol; Poloxamer; Poloxamer 407; poly(ethylene glycol) (12) tridecyl ether mixture of C11 to C14 iso-alkyl ethers with C13 iso-alkyl predominating; poly(ethylene glycol) (18) tridecyl ether mixture of C11 to C14 iso-alkyl ethers with C13 iso-alkyl predominating; poly(ethylene glycol) sorbitan tetraoleate; poly(ethylene glycol) sorbitol hexaoleate; polyethoxylated tallow amine; polyethylene glycol dodecyl ether; polyethylene glycol esters; polyethylene-block-poly(ethylene glycol) average Mn ~1,400; polyethylene-block-poly(ethylene glycol) average Mn ~575; polyethylene-block-poly(ethylene glycol) average Mn ~875; polyethylene-block-poly(ethylene glycol) average Mn ~920; polyglycerol polyricinoleate; polyoxyethylene fatty acid amides; polyoxyethylene surfactants; Polysorbate; Polysorbate 20; Polysorbate 80; sorbitan; sorbitan monolaurate (Span 20); sorbitan monopalmitate (Span 40); sorbitan monostearate (Span 60); sorbitan monooleate (Span 80); sorbitan sesquioleate (Span 83); sorbitan trioleate (Span 85); sorbitan isostearate (Span 120); SP Brij® C2 MBAL-SO-(SG); SP Brij® C2 MBAL-SO-(SG); SP Brij® S2 MBAL; SPAN 20; stearyl alcohol; Surfactin; Triton N-101; Triton X-100; Triton X-100; Triton X-114; Triton X-405; Tween® 20; Tween® 40; Tween® 60; Tween® 80; and Tween® 85.

In some embodiments, the nonionic surfactant is a polyalkylene glycol alkyl ether.

In some embodiments, the nonionic surfactant may be present at up to about 20% w/w, such as 0.1, 0.15, 0.2, 0.3, 0.4, 0.5, 1.0, 1.5, 2, 2.5, 3, 3.5, 4, 4.5, 5, 5.5, 6, 6.5, 7, 8, 9, 19, 12, 14, 16, 18 or 20% w/w or about 0.1, 0.15, 0.2, 0.3, 0.4, 0.5, 1.0, 1.5, 2, 2.5, 3, 3.5, 4, 4.5, 5, 5.5, 6, 6.5, 7, 8, 9, 19, 12, 14, 16, 18 or 20% w/w. In some embodiments, the nonionic surfactant is present at up to about 10% w/w, such as 0.1, 0.15, 0.2, 0.3, 0.4, 0.5, 1.0, 2.0, 3.0, 4.0, 5.0, 6.0, 7.0, 8.0, 9.0 or 10.0% w/w or about 0.1, 0.15, 0.2, 0.3, 0.4, 0.5, 1.0, 2.0, 3.0, 4.0, 5.0, 6.0, 7.0, 8.0, 9.0 or 10.0% w/w. In some embodiments, the nonionic surfactant is present at up to about 5% w/w, such as 0.1, 0.15, 0.2, 0.3, 0.4, 0.5, 1.0, 2.0, 3.0, 4.0, 5.0% w/w or about 0.1, 0.15, 0.2, 0.3, 0.4, 0.5, 1.0, 2.0, 3.0, 4.0, 5.0% w/w.

In some embodiments, the polyalkylene glycol alkyl ether is a polypropylene oxide alkyl ether or a polyethylene glycol alkyl ether. Some non-limiting examples of polyalkylene glycol alkyl ethers include poly(oxyethylene) cetyl ether, poly(oxyethylene) palmityl ether, polyethylene oxide hexadecyl ether, polyethylene glycol cetyl ether, Brij 30 (Brij L4), Brij 38, Brij 52, Brij 56, Brij 58, Brij 78, Brij 98, Brij 700, Brij 700P, Brij 721, Brij S20, and Brij W1. In some embodiments, the polyalkylene glycol alkyl ether is a Brij group polyalkylene glycol alkyl ether. In some embodiments, the polyalkylene glycol alkyl ether is Brij L4.

### Pressure Sensitive Adhesive

In some embodiments, the composition includes at least one pharmaceutically acceptable pressure sensitive adhesive.

In some embodiments, the pressure sensitive adhesive is a pressure sensitive adhesive based on polyacrylate. In some embodiments, said pressure sensitive adhesive based on polyacrylate forms a matrix in which is embedded the active ingredient. In some embodiments, the pressure sensitive adhesives are polyacrylate based and available commercially, for example the Gelva and Durotak brands, especially the series 87 and GMS, for example DURO-TAK 87-900A, DURO-TAK 87-9301, DURO-TAK 87-4098, GELVA GMS 3083, DURO-TAK 387-2510 / 87-2510, DURO-TAK 387-2287 / 87-2287, DURO-TAK 87-4287, GELVA GMS 788, DURO-TAK 387-2516 / 87-2516, DURO-TAK 87-2074, DURO-TAK 87-235A, DURO-TAK 387-2353 / 87-2353, GELVA GMS 9073, DURO-TAK 87-2852, DURO-TAK 387-2051 / 87-2051, DURO-TAK 387-2052 / 87-2052, DURO-TAK 387-2054 / 87-2054, DURO-TAK 87-2194, DURO-TAK 87-2196.

The pressure sensitive adhesive based on polyacrylate may contain one or more acrylate homopolymers or copolymers of one or more acrylate or any combination or mixture thereof.

In some embodiments, the acrylate pressure sensitive adhesive is DURO-TAK UN1133, DURO-TAK 387-2516, DURO-TAK 87-4098, DURO-TAK 387-2054 or GELVA GMS 7883.

### MolAccs

In some embodiments, the compositions and formulations of the present invention may include one or more MolAccs. Examples of molecular entities that serve as MolAccs include, but are not limited to lower monohydric alcohols, lower terpenes, lower fatty acids, and DMSO.

DMSO is a polar aprotic solvent characterized as having low surface tension. DMSO permeates readily through skin and is known to function in some instances as a penetration enhancer.

In some embodiments, the MolAcc may be present in an amount between 0% and 99% w/w or about 0% and about 99% w/w. In some embodiments, the MolAcc is present in an amount of 0%, 5%, 10%, 15%, 20%, 25%, 30%, 35%, 40%, 45%, 50%, 55%, 60%, 65%, 70%, 75%, 80%, 85%, 90%, 95%, or 99% w/w or about 0%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% w/w or any amount within a range defined by any two of the aforementioned numbers.

In some embodiments, the MolAcc has a secondary function in the composition, including but not limited to a solvent, MPE, solubilizing agent, antioxidant, emollient or preservative.

### Molecular Penetration Enhancers (“MPE”)

In some embodiments, the compositions and formulations for the present invention may include one or more MPEs. Examples of MPEs include, but are not limited to 1,3-butanediol, alpha-tocopherol, ceteth-2, coco-caprylate/caprate, cocodiethanolamide, diethanolamine, diethylsebacate, dimethyl sulfoxide, dipropylene glycol, ethyl acetate, ethyl oleate, ethylene glycol, glycerol, hexylene glycol, isopropyl alcohol, Labrasol®, lactic acid, laureth-2, lauric diethanolamide, lauryl lactate, levulinic acid, L-menthol, oleic acid, oleth-5, oleyl alcohol, propylene glycol, steareth-20 and Transcutol.

In some embodiments, the MPE has a dual role including but not limited to a MolAcc.

### Emollients

Emollients can optionally be added to the formulations of the invention so that the formulations can maintain or increase the moisture content of the stratum corneum when the composition is applied to the skin. Emollients may be added to the formulations in addition to the components already described, which may also aid in maintaining or improving the skin condition of the user.

In some embodiments, added emollients are included in the compositions of the invention at a concentration between 0% and 99% w/w or about 0% and about 99% w/w, such as 0%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% w/w, or about 0%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% w/w or any concentration within a range defined by any two of the aforementioned values.

Example emollients may be selected from any of the classes known in the art. A general list of useful emollients appears, for example, in U.S. Pat. No. 4,478,853 and in EP patent application 0 522 624A1 as well as in the CTFA Cosmetic Ingredient Handbook published by The Cosmetic, Toiletry, and Fragrance Association, Washington D.C. (1992) under listings including but not limited to “Skin Conditioning agents”, “emollients”, “humectants”, “miscellaneous” and “occlusive.”

In some embodiments, the addition of one or more emollients may affect the viscosity and stability of the compositions of the present invention. In some embodiments, a single emollient may be added to the composition. In other embodiments, two or more emollients may be added to the composition. While any of a variety of emollients may be added to the formulations of the present invention, some embodiments will include wax and oil type emollients either alone or combined with water soluble emollients. In some embodiments of the invention, emollient systems can be comprised of humectants in addition to occlusive wax and oil emollients in concentrations that achieve a moisturizing effect and which maintain and improve the condition of the skin upon repeated use. Emollients may be non-comedogenic and chosen to avoid skin irritation or sensitization reactions.

### Solubilizing Agent

In some embodiments, the formulations and compositions of the present invention may include a solubilizing agent. Examples of solubilizing agents include, but are not limited to: 2-hydroxypropyl-β-cyclodextrin; Cremophor EL; dimethylsulfoxide; docusate sodium; ethanol; Gelucire 44/14; Labrasol; Nonoxynol 9; Octoxymol 9; PEG-60 Hydrogenated Castor Oil (HCO-60); Poloxamer 124; Poloxamer 188; Poloxamer 237; Poloxamer 338; Poloxamer 407; Poloxamer; Polyethylene glycol 400 (PEG 400); Polyoxyl 10 Oleyl Ether; Polyoxyl 20 Cetostearyl Ether; Polyoxyl 35 Castor Oil; Polyoxyl 40 Hydrogenated Castor Oil; Polyoxyl 40 Stearate; Polysorbate 20; Polysorbate 40; Polysorbate 60; Polysorbate 80; propylene glycol; sodium lauryl sulfate; sodium taurocholate; sorbitan monolaurate; sorbitan monooleate; sorbitan monopalmitate; sorbitan monostearate; sulfobutylether-β-cyclodextrin (Captisol); Transcutol P; Tween 80; Tyloxapol.

### Antioxidant

In some embodiments, the formulation additionally comprises an anti-oxidant. Example anti-oxidants include but are not limited to ascorbic acid, ascorbyl linoleate, ascorbyl dipalmitate, ascorbyl palmitate, ascorbyl tocopherol maleate, butylated hydroxytoluene, butylated hydroxyanisole (BHA), calcium ascorbate, carotenoids, kojic acid and its pharmaceutically acceptable salts, propyl gallate, sodium thiosulfate, thioglycolic acid and its pharmaceutically acceptable salts (e.g., ammonium), tocopherol (including α, β, γ and δ forms), tocopherol acetate, tocophereth-5, tocophereth-12, tocophereth-18, or tocophereth-80.

### Preservative

In some embodiments, the formulation additionally comprises at least one preservative. Example preservatives include but are not limited to benzalkonium chloride, cetrimonium bromide (aka cetyltrimethylammonium bromide), cetylpyridinium chloride, benzethonium chloride, alkyltrimethylammonium bromide, methyl paraben, ethyl paraben, propyl paraben, butyl paraben, benzyl alcohol, cetyl alcohol, steryl alcohol, benzoic acid, sorbic acid, chloroacetamide, trichlorocarban, thimerosal, imidurea, bronopol, chlorhexidine, 4-chlorocresol, 4-chloroxylenol, dichlorophene and hexachlorophene. Especially preferred are cetylpyridinium chloride, methyl paraben and propyl paraben, or mixtures thereof.

### Chelating Agent

Chelating agents form complexes with metal ions, for example, to improve stability of a composition comprising metals or to improve excretion of metal ions. In some embodiments, the formulation comprises at least one chelating agent. Example chelating agents include but are not limited to ethylenediaminetetraacetic acid (“EDTA”), disodium edetate, sodium calcium edetate, dimercaprol, succimer, 2,3-dimercapto-1-propanesulfonic acid, alpha lipoic acid, citric acid, phosphonates, or porphyrins.

### Organic Solvents

In some embodiments, the compositions of the present invention are formulated with organic solvents. Examples of organic solvents include but are not limited to acetic acid; acetone; acetonitrile; 1-butanol; 2-butanol; 2-butanone; tert-butyl alcohol; cyclohexane; diethylene glycol; diethyl ether; diglyme (diethylene glycol); dimethyl ether; dimethyl isosorbide; 1,2-dimethoxy-ethane (glyme or “DME”); dimethylformamide (“DMF”); DMSO; 1,4-dioxane; ethanol; ethyl acetate; ethylene glycol; glycerin; heptane; Hexamethylphosphoramide (HMPA); Hexamethylphosphorous triamide (HMPT); hexane; methanol; methyl t-butyl ether (MTBE); methylene chloride; N-methyl-2-pyrrolidinone (“NMP”); nitromethane; pentane; petroleum ether (ligroine); 1-propanol; 2-propanol; pyridine; tetrahydrofuran (“THF”); toluene; triethylamine; o-xylene; m-xylene; p-xylene. In some embodiments, organic solvents for use in the compositions are substances that are pharmaceutically acceptable for application to the skin. In some embodiments, the compositions include at least two organic solvents. In some embodiments, the formulations may have different volatilities. In some embodiments, one of the solvents is highly volatile such that the formulation substantially dries relatively quickly on application to the skin of a subject while the second solvent is less volatile and serves to maintain the lidocaine and/or congener thereof in a substantially solubilized form in order that the lidocaine and/or congener thereof can continue to be efficiently delivered into the skin of the subject. In some embodiments, one of the solvents is highly volatile such that the formulation substantially dries when applied to a suitable substrate, such as a polymeric backing film, to provide an adhesive film suitable for later application to the skin of a subject while the second solvent is less volatile and serves to maintain the lidocaine and/or congener thereof in a substantially solubilized form in the film in order that the lidocaine can continue to be efficiently delivered into the skin of the subject.

Some embodiments provided herein relate to topical compositions. In some embodiments, the compositions include a topical anesthetic, at least one monohydric alcohol, a terpene or terpenoid, at least one isopropyl ester of a fatty acid, and at least one monohydric alcohol.

In some embodiments, the compositions include at least one topical anesthetic, each present in a weight percent ranging from 1 to 99% w/w, such as 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, or 99% w/w, or in a percent within a range defined by any two of the aforementioned values. In other embodiments, the at least one topical anesthetic includes lidocaine.

In some embodiments, the compositions include at least one monohydric alcohol, each present in a weight percent ranging from 1 to 99% w/w, such as 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, or 99% w/w, or in a percent within a range defined by any two of the aforementioned values. In some embodiments, the at least one monohydric alcohol includes at least one C2-C4 monohydric alcohol. In some embodiments, the at least one C2-C4 monohydric alcohol includes ethanol.

In some embodiments, the compositions include at least one terpene or terpenoid, each present in a weight percent ranging from 1 to 99% w/w, such as 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, or 99% w/w, or in a percent within a range defined by any two of the aforementioned values. In some embodiments, the at least one terpene or terpenoid includes limonene.

In some embodiments, the compositions include at least one isopropyl ester of a fatty acid, each present in a weight percent ranging from 1 to 99% w/w, such as 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, or 99% w/w, or in a percent within a range defined by any two of the aforementioned values. In some embodiments, the at least one isopropyl ester of a fatty acid includes at least one isopropyl ester of a C14-C18 fatty acid. In some embodiments, the at least one isopropyl ester of a C14-C18 fatty acid includes isopropyl myristate and/or isopropyl palmitate.

In some embodiments, the compositions include at least one monohydric alcohol, each present in a weight percent ranging from 1 to 99% w/w, such as 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, or 99% w/w, or in a percent within a range defined by any two of the aforementioned values. In some embodiments, the at least one monohydric C14-C20 alcohol includes at least one monohydric C14-C20 alcohol. In some embodiments, the at least one monohydric C14-C20 alcohol includes isostearyl alcohol.

In some embodiments, the compositions additionally include at least one nonionic surfactant, each present in a weight percent ranging from 1 to 99% w/w, such as 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, or 99% w/w, or in a percent within a range defined by any two of the aforementioned values. In some embodiments, the at least one nonionic surfactant includes polyethylene glycol dodecyl ether.

In some embodiments, the compositions additionally include at least one MolAcc, each present in a weight percent ranging from 1 to 99% w/w, such as 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, or 99% w/w, or in a percent within a range defined by any two of the aforementioned values. In some embodiments, the at least one MolAcc includes DMSO.

In some embodiments, the compositions additionally include at least one MPE, each present in a weight percent ranging from 1 to 99% w/w, such as 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, or 99% w/w, or in a percent within a range defined by any two of the aforementioned values.

In some embodiments, the compositions additionally include at least one emollient, each present in a weight percent ranging from 1 to 99% w/w, such as 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, or 99% w/w, or in a percent within a range defined by any two of the aforementioned values.

In some embodiments, the compositions additionally include at least one solubilizing agent, each present in a weight percent ranging from 1 to 99% w/w, such as 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, or 99% w/w, or in a percent within a range defined by any two of the aforementioned values.

In some embodiments, the compositions additionally include at least one antioxidant, each present in a weight percent ranging from 1 to 99% w/w, such as 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, or 99% w/w, or in a percent within a range defined by any two of the aforementioned values.

In some embodiments, the compositions additionally include at least one preservative, each present in a weight percent ranging from 1 to 99% w/w, such as 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, or 99% w/w, or in a percent within a range defined by any two of the aforementioned values.

In some embodiments, the compositions additionally include at least one chelating agent, each present in a weight percent ranging from 1 to 99% w/w, such as 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, or 99% w/w, or in a percent within a range defined by any two of the aforementioned values.

In some embodiments, the compositions additionally include at least one organic solvent, each present in a weight percent ranging from 1 to 99% w/w, such as 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, or 99% w/w, or in a percent within a range defined by any two of the aforementioned values.

Some embodiments provided herein relate to methods of preventing local pain in a subject by administering a therapeutically effective amount of the topical formulations described herein to the subject. The topical formulation is applied to the subject for 1, 5, 10, 15, 20, 25, 30, 35, 40, 50, 55, or 60 seconds, or 1, 2, 3, 4, 5, 10, 15, 20, 25, 30, 35, 40, 45, 50, 60 minutes, or 1, 2, 3, 4, 5 hours, or for a duration within a range defined by any two of the aforementioned values, or until the subject is sufficiently relieved of local pain.

Some embodiments provided herein relate to a device suitable for providing rapid local anesthesia using the topical formulations described herein. The device comprises a pressure-sensitive adhesive formed into an annulus, circle, square, rectangle, polygon, or any other appropriate shape to cover the area to be affected, wherein the topical formulations described herein are applied to the inner circumference of the annulus, or any other appropriate region of the pressure-sensitive adhesive.

### Non-limiting Formulations

Disclosed herein are various embodiments of topical formulations for rapid onset topical anesthesia. The embodiments may include any one of the active agents (such as one or more aminoamide or aminoester anesthetics) as well as one or more excipients disclosed herein or otherwise known in the art for various purposes including but not limited to improving permeability across the skin, increasing duration of the effect by releasing the active agent at a reduced rate, improving the stability of the formulation, either at the time of use or in storage, increasing the viscosity of the formulation for improved handling, or providing adhesive properties to the formulation. Non-limiting embodiments of the topical formulations are provided throughout the disclosure, and particularly in the Examples. It is envisioned that the described ratios for any one or more constituent compounds of each formulation may be adjusted within a reasonable range, for example, increasing or decreasing the relative % w/w of a compound by 0.5%, 1 %, 2%, 3%, 4%, 5%, 6%, 7%, 8%, 9%, or 10% w/w, or any % w/w within a range defined by any two of the aforementioned percentages.

Disclosed herein are topical formulations comprising lidocaine present in an amount of 1% to 10% or about 1% to about 10% w/w (for example, about 1%, 2%, 3%, 4%, 5%, 6%, 7%, 8%, 9%, or 10% w/w), limonene present in an amount of 5% to 20% or about 5% to about 20% w/w (for example, about 5%, 6%, 7%, 8%, 9%, 10%, 11%, 12%, 13%, 14%, 15%, 16%, 17%, 18%, 19%, or 20% w/w), and at least one C2-C4 monohydric alcohol present in an amount of 40% to 60% or about 40% to about 60% w/w (for example, about 40%, 41%, 42%, 43%, 44%, 45%, 46%, 47%, 48%, 49%, 50%, 51%, 52%, 53%, 54%, 55%, 56%, 57%, 58%, 59%, or 60% w/w). In some embodiments, the at least one C2-C4 monohydric alcohol comprises ethanol. In some embodiments, the at least one C2-C4 monohydric alcohol is ethanol. In some embodiments, the lidocaine is present in an amount of 4% or about 4% w/w. In some embodiments, the limonene is present in an amount of 10% or about 10% w/w. In some embodiments, the at least one C2-C4 monohydric alcohol is present in an amount of 51% or about 51% w/w. In some embodiments, the lidocaine may be substituted for any one or more other active agents, such as another aminoamide or aminoester anesthetic. In some embodiments, the limonene may be substituted for any one or more other molecular penetration enhancers or terpenes. In some embodiments, the at least one C2-C4 monohydric alcohol may be substituted for any one or more other C2-C4 monohydric alcohols.

In some embodiments, the topical formulations disclosed herein further comprise at least one isopropyl ester of a C14-C18 fatty acid. In some embodiments, the at least one isopropyl ester of a C14-C18 fatty acid is present in an amount of 10% to 30% or about 10% to about 30% w/w (for example, about 10%, 11%, 12%, 13%, 14%, 15%, 16%, 17%, 18%, 19%, 20%, 21%, 22%, 23%, 24%, 25%, 26%, 27%, 28%, 29% or 30% w/w). In some embodiments, the at least one isopropyl ester of a C14-C18 fatty acid comprises isopropyl palmitate or isopropyl myristate, or both. In some embodiments, the isopropyl palmitate and the isopropyl myristate are each present in an amount of 5% to 15% or about 5% to about 15% w/w (for example, about 5%, 6%, 7%, 8%, 9%, 10%, 11%, 12%, 13%, 14%, or 15% w/w). In some embodiments, the isopropyl palmitate is present in an amount of 7% or about 7% w/w. In some embodiments, the isopropyl myristate is present in an amount of 11%, or about 11% w/w. In some embodiments, the lidocaine is present in an amount of 4% or about 4% w/w, the limonene is present in an amount of 10% or about 10% w/w, the at least one C2-C4 monohydric alcohol is present in an amount of 51% or about 51% w/w, the isopropyl palmitate is present in an amount of 7% or about 7% w/w, and the isopropyl myristate is present in an amount of 11% or about 11% w/w. In some embodiments, the at least one isopropyl ester of a C14-C18 fatty acid may be substituted for any one or more other isopropyl ester of a C14-C18 fatty acid.

In some embodiments, the topical formulations disclosed herein further comprise at least one monohydric C14-C20 alcohol. In some embodiments, the at least one monohydric C14-C20 alcohol is present in an amount of 5% to 15% or about 5% to about 15% w/w (for example, about 5%, 6%, 7%, 8%, 9%, 10%, 11%, 12%, 13%, 14%, or 15% w/w). In some embodiments, the at least one monohydric C14-C20 alcohol comprises isostearyl alcohol. In some embodiments, the at least one monohydric C14-C20 alcohol is isostearyl alcohol. In some embodiments, the isostearyl alcohol is present in an amount of 10% or about 10% w/w. In some embodiments, the lidocaine is present in an amount of 4% or about 4% w/w, the limonene is present in an amount of 10% or about 10% w/w, the at least one C2-C4 monohydric alcohol is present in an amount of 51% or about 51% w/w, the isopropyl palmitate is present in an amount of 7% or about 7% w/w, the isopropyl myristate is present in an amount of 11% or about 11% w/w, and the at least one monohydric C14-C20 alcohol is present in an amount of 10% or about 10% w/w. In some embodiments, the at least one monohydric C14-C20 alcohol may be substituted for any one or more other monohydric C14-C20 alcohols.

In some embodiments, the topical formulations disclosed herein further comprise at least one polyalkylene glycol alkyl ether. In some embodiments, the at least one polyalkylene glycol alkyl ether is present in an amount of 0% to 10% or about 0% to about 10% w/w (for example, about 0%, 1%, 2%, 3%, 4%, 5%, 6%, 7%, 8%, 9%, or 10% w/w). In some embodiments, the at least one polyalkylene glycol alkyl ether comprises polyethylene glycol dodecyl ether. In some embodiments, the polyethylene glycol dodecyl ether is present in an amount of 5% or about 5% w/w. In some embodiments, the lidocaine is present in an amount of 4% or about 4% w/w, the limonene is present in an amount of 10% or about 10% w/w, the at least one C2-C4 monohydric alcohol is present in an amount of 51% or about 51% w/w, the isopropyl palmitate is present in an amount of 7% or about 7% w/w, the isopropyl myristate is present in an amount of 11% or about 11% w/w, the at least one monohydric C14-C20 alcohol is present in an amount of 10% or about 10% w/w, and the at least one polyalkylene glycol alkyl ether is present in an amount of 5% or about 5% w/w. In some embodiments, the at least one polyalkylene glycol alkyl ether may be substituted for any one or more other polyalkylene glycol alkyl ethers.

In some embodiments, the topical formulations disclosed herein further comprise a cellulosic thickening agent. In some embodiments, the cellulosic thickening agent is present in an amount of 0% to 5% or about 0% to about 5% w/w (for example, about 0%, 1%, 2%, 3%, 4%, or 5% w/w). In some embodiments, the cellulosic thickening agent comprises hydroxypropyl cellulose. In some embodiments, the hydroxypropyl cellulose is present in an amount of 2% or about 2%. In some embodiments, the lidocaine is present in an amount of 4% or about 4% w/w, the limonene is present in an amount of 10% or about 10% w/w, the at least one C2-C4 monohydric alcohol is present in an amount of 51% or about 51% w/w, the isopropyl palmitate is present in an amount of 7% or about 7% w/w, the isopropyl myristate is present in an amount of 11% or about 11% w/w, the at least one monohydric C14-C20 alcohol is present in an amount of 10% or about 10% w/w, the at least one polyalkylene glycol alkyl ether is present in an amount of 5% or about 5% w/w, and the cellulosic thickening agent is present in an amount of 2% or about 2%. In some embodiments, the cellulosic thickening agent may be substituted for any one or more other thickening agents.

Also disclosed herein are topical formulations comprising lidocaine present in an amount of 1% to 10% or about 1% to about 10% w/w (for example, about 1%, 2%, 3%, 4%, 5%, 6%, 7%, 8%, 9%, or 10% w/w), a pressure sensitive adhesive present in an amount of 70% to 90% or about 70% to about 90% w/w (for example, about 70%, 71%, 72%, 73%, 74%, 75%, 76%, 77%, 78%, 79%, 80%, 81%, 82%, 83%, 84%, 85%, 86%, 87%, 88%, 89%, or 90% w/w), a molecular penetration enhancer present in an amount of 0% to 5% or about 0% to about 5% w/w (for example, about 0%, 1%, 2%, 3%, 4%, or 5% w/w), and at least one polyalkylene glycol alkyl ether present in an amount of 0% to 5% or about 0% to about 5% w/w (for example, about 0%, 1%, 2%, 3%, 4%, or 5% w/w). In some embodiments, the lidocaine is present in an amount of 4% or about 4% w/w. In some embodiments, the pressure sensitive adhesive is present in an amount of 83% or about 83% w/w. In some embodiments, the pressure sensitive adhesive comprises DURO-TAK 87-4098. In some embodiments, the molecular penetration enhancer is present in an amount of 3% or about 3% w/w. In some embodiments, the molecular penetration enhancer comprises levulinic acid. In some embodiments, the at least one polyalkylene glycol alkyl ether is present in an amount of 2% or about 2%. In some embodiments, the at least one polyalkylene glycol alkyl ether comprises polyethylene glycol dodecyl ether. In some embodiments, the at least one polyalkylene glycol alkyl ether is polyethylene glycol dodecyl ether. In some embodiments, the lidocaine may be substituted for any one or more other active agents, such as another aminoamide or aminoester anesthetic. In some embodiments, the pressure sensitive adhesive may be substituted for any one or more other pressure sensitive adhesives. In some embodiments, the molecular penetration enhancer may be substituted for any one or more other molecular penetration enhancers. In some embodiments, the at least one polyalkylene glycol alkyl ether may be substituted for any one or more other polyalkylene glycol alkyl ethers.

In some embodiments, the topical formulations disclosed herein further comprise at least one isopropyl ester of a C14-C18 fatty acid. In some embodiments, the at least one isopropyl ester of a C14-C18 fatty acid is present at an amount of 5% to 10% or about 5% to about 10% w/w (for example, about 5%, 6%, 7%, 8%, 9%, or 10% w/w). In some embodiments, the at least one isopropyl ester of a C14-C18 fatty acid comprises isopropyl palmitate or isopropyl myristate, or both. In some embodiments, the isopropyl palmitate and the isopropyl myristate are each present in an amount of 2.5% to 5% or about 2.5% to about 5% w/w (for example, about 2.5%, 3%, 3.5%, 4%, 4.5%, or 5% w/w). In some embodiments, the isopropyl palmitate or the isopropyl myristate, or both, are present in an amount of 4% or about 4% w/w. In some embodiments, the lidocaine is present in an amount of 4% or about 4% w/w, the pressure sensitive adhesive is present in an amount of 83% or about 83% w/w, the molecular penetration enhancer is present in an amount of 3% or about 3% w/w, the polyalkylene glycol alkyl ether is present in an amount of 2% or about 2% w/w, the isopropyl palmitate is present in an amount of 4% or about 4% w/w, and the isopropyl myristate is present in an amount of 4% or about 4% w/w. In some embodiments, the at least one isopropyl ester of a C14-C18 fatty acid may be substituted for any one or more other isopropyl esters of a C14-C18 fatty acid.

In some embodiments of the present application, a pharmaceutical composition comprises lidocaine, ethanol, limonene, isopropyl palmitate, isopropyl myristate, and isostearyl alcohol.

According to some embodiments disclosed herein, there is provided a pharmaceutical composition comprising, consisting essentially of, or consisting of:
about 4% w/w lidocaine;
about 30% w/w to about 65% w/w ethanol;
about 10% w/w limonene;
about 4% w/w to about 15% w/w isopropyl myristate;
about 4% w/w to about 15% w/w isopropyl palmitate;
about 5% w/w to about 15% w/w of isostearyl alcohol.

In some embodiments, there is further included in said pharmaceutical composition: about 1% w/w to about 8% w/w polyethylene glycol dodecyl ether. In some embodiments, there is further included in said pharmaceutical composition: about 1% w/w to about 5% w/w hydroxypropyl cellulose.

In some embodiments, there is provided a pharmaceutical composition comprising lidocaine, a C2-C4 monohydric alcohol, limonene, an isopropyl ester of a C14-C18 fatty acid, and a monohydric C 14-C20 alcohol.

According to some embodiments disclosed herein, there is provided a pharmaceutical composition comprising, consisting essentially of, or consisting of:
about 1% w/w to about 5% w/w lidocaine;
about 30% w/w to about 65% w/w of a C2-C4 monohydric alcohol;
about 2% w/w to about 20% w/w of limonene;
about 2% w/w to about 20% w/w of an isopropyl ester of a C14-C18 fatty acid;
about 2% w/w to about 15% w/w of a monohydric C14-C20 alcohol

In some embodiments, there is further included in said pharmaceutical composition: about 1% w/w to about 10% w/w polyethylene glycol dodecyl ether. In some embodiments, there is further included in said pharmaceutical composition: about 1% w/w to about 5% w/w hydroxypropyl cellulose.

In some embodiments of the present application, there is provided a pharmaceutical composition comprising lidocaine, a C2-C4 monohydric alcohol, limonene, the isopropyl esters of two C14-C18 fatty acids, and a monohydric C14-C20 alcohol.

According to some embodiments disclosed herein, there is provided a pharmaceutical composition comprising, consisting essentially of, or consisting of:
about 1% w/w to about 5% w/w lidocaine;
about 30% w/w to about 65% w/w of a C2-C4 monohydric alcohol;
about 2% w/w to about 20% w/w of limonene;
about 2% w/w to about 15% w/w of the isopropyl ester of a first C14-C18 fatty acid;
about 2% w/w to about 15% w/w of the isopropyl ester of a second C14-C18 fatty acid;
about 2% w/w to about 15% w/w of a monohydric C14-C20 alcohol

In some embodiments, there is further included in said pharmaceutical composition: about 1% w/w to about 10% w/w polyethylene glycol dodecyl ether. In some embodiments, there is further included in said pharmaceutical composition: about 1% w/w to about 5% w/w hydroxypropyl cellulose.

In some embodiments, there is provided a pharmaceutical composition suitable for topical administration comprising an active agent, a C2-C4 monohydric alcohol, limonene, the isopropyl esters of two C14-C18 fatty acids, and a monohydric C14-C20 alcohol.

According to some embodiments disclosed herein, there is provided a pharmaceutical composition comprising, consisting essentially of, or consisting of:
about 0.01% w/w to about 25% w/w of an active agent;
about 30% w/w to about 65% w/w of a C2-C4 monohydric alcohol;
about 2% w/w to about 20% w/w of limonene;
about 2% w/w to about 15% w/w of the isopropyl ester of a first C14-C18 fatty acid;
about 2% w/w to about 15% w/w of the isopropyl ester of a second C14-C18 fatty acid;
about 2% w/w to about 15% w/w of a monohydric C14-C20 alcohol

In some embodiments, there is further included in said pharmaceutical composition: about 1% w/w to about 10% w/w polyethylene glycol dodecyl ether. In some embodiments, there is further included in said pharmaceutical composition: about 1% w/w to about 5% w/w hydroxypropyl cellulose.

### Characteristics of Compositions Disclosed Herein

### (A) Delivery of the Active Agent

In some embodiments, the composition provides substantially reduced onsets relative to comparative formulations. This substantially reduced onset derives from dual benefits of the inventive composition, namely substantially reduced lag times combined with substantially accelerated delivery kinetics relative to comparative formulations.

### (B) Stability

The stability of a drug product composition can have a significant impact on the length and cost of drug development, the nature of the studies required to support regulatory submissions, and the ultimate safety and approvability.

In some embodiments, the topical formulations disclosed herein exhibit chemical and physical characteristics suitable for clinical development and commercialization.

In some embodiments, there is provided a lidocaine formulation wherein the lidocaine degrades by less than 5% over the course of 6 months at room temperature. More preferably, the rate of degradation is less than 5.0, 4.0, 3.0, 2.0, 1.0, 0.9, 0.8, 0.7, 0.6, 0.5, 0.4, 0.3, 0.2, or less than 0.1%, and all fractions in between, over the course of 6 months at room temperature.

In some embodiments, the composition remains stable for an acceptable time period between preparation and use when stored in a closed container at normal ambient temperature. In some embodiments, an “acceptable time period” is at least about 30 days, at least about six months, at least about one year, or at least about two years.

In some embodiments, the composition maintains at least 75%, 80%, 85%, 90% or 95% strength of the active ingredient following storage for two weeks at 25° C. In some embodiments, the composition maintains at least 75%, 80%, 85%, 90% or 95% strength of the active ingredient following storage for two weeks at 40° C. In some embodiments, the composition maintains at least 75%, 80%, 85%, 90% or 95% strength of the active ingredient following storage for 16 days at 40° C. In some embodiments, the composition maintains at least 75%, 80%, 85%, 90% or 95% strength of the active ingredient following storage for 43 days at 40° C.

### (C) Pharmacokinetic Properties

In some embodiments, the composition provides substantially reduced onsets relative to comparative formulations. This substantially reduced onset derives from dual benefits of the inventive composition, namely substantially reduced lag times combined with substantially accelerated delivery kinetics relative to comparative formulations. In some embodiments, the compositions provide onsets of less than one hour, for example, within 5, 10, 15, 20, 25, 30, 35, 40, 45, 50, 55, or 60 minutes, or any time of onset within a range defined by any two of the aforementioned times.

In some embodiments, following topical administration the composition provides substantially increased proportionate delivery into viable epidermal and dermal tissue relative to transdermal permeation, compared with comparator formulations.

In some embodiments, the composition provides no more than minor skin irritation, evidenced by a score in a Draize test in albino rabbit skin or human volunteers of no more than 2 in erythema, no more than 2 in edema, and no more than 2 in combined Draize score.

### (D) Viscosity

In some embodiments, the composition of the present application is more viscous than water at standard temperature and pressure (“STP”). In some embodiments, the composition has a kinematic viscosity of more than about 1 centistokes (“cSt”) or a dynamic viscosity of more than about 1 centipoise (cP). In some embodiments, the dynamic viscosity of the composition is at most about 2, 3, 4, 5, 7, 10, 12, 15, 20, 25, 30, 35, 40, 45, 50, 60, 70, 75, 80, 90, 100, 150, 200, 250, 500, 1,000, 2,000, 3,000, 5,000, 10,000, 20,000, 50,000, 100,000, 200,000, 500,000 or 1,000,000 cP at STP. In some embodiments, the dynamic viscosity is at most about 2, 3, 4, 5, 7, 10, 12, 15, 20, 25, 30, 35, 40, 45 or 50 cP at STP. In some embodiments, the dynamic viscosity is at most about 2, 3, 4, 5, 7, 10, 12, 15 or 20 cP at STP. In some embodiments, the composition is thixotropic (i.e., it decreases in viscosity upon being stirred or shaken). The composition’s viscosity can be adjusted by the addition of a thickening agent, such as a cellulosic thickening agent, for example, hydroxypropyl cellulose, or other thickening agents, or mixtures thereof.

In some embodiments the composition is provided in the form of a topical patch.

In some embodiments, the composition is provided in the form of a viscous gel, suitable for provision in the reservoir of a topical patch of a reservoir design.

In some embodiments, the composition is provided in the form of a viscous gel, suitable for within an annulus of pressure-sensitive adhesive.

### Methods of Preparation

In embodiments of the present application, the pharmaceutical compositions are formulated as a spray, a cream, a lotion, an emulsion, a microemulsion, a gel, a lacquer, an ointment, a solution, or a patch, film or mask for topical administration. In a suitable embodiment, the composition is a topical patch.

Methods of preparing compositions for transdermal administration are known in the art (see, for example, Remington’s Pharmaceutical Sciences, 2000-20th edition, and The United States Pharmacopeia: The National Formulary, USP 24 NF19, published in 1999).

In some embodiments, the formulation is prepared in the form of a topical film or patch. Appropriate amounts of the liquid ingredients as taught herein are added to an amount of lidocaine in a container and appropriate amounts of the chosen acrylate copolymer solution or solutions are added. The container is covered to prevent evaporative losses and the container contents are intimately mixed. Any air bubbles in the resulting homogeneous solution are removed. The resulting solution is applied to a backing film and spread over the surface of a suitable substrate, such as a backing film, a release film or a transfer film, to provide a formulation film of a suitable uniform thickness. The application and spreading process may be achieved manually. For preparing larger amounts of film, a semi-automated or automated process is preferred, as is well-known in the art. After the film has been deposited, it is subjected to a controlled period of heating to evaporate the volatile solvents. Conveniently, another substrate, such as a release film, a backing film, or a transfer film is applied to the exposed formulation film surface to protect the formulation prior to use. Conveniently, the film composite is then sealed in a suitable pouch container to further protect the film from dirt, air, moisture and other possible contaminants.

### Methods of Treatment

In some embodiments, the invention describes a method for providing local anesthesia in a subject comprising the step of applying a therapeutically effective amount of a topical formulation to a subject to provide a topical anesthetic intradermally. In some non-limiting embodiments, the topical anesthetic is lidocaine. In some embodiments, the topical formulation is any one of the topical formulations disclosed herein.

In some embodiments, the pharmaceutical composition is applied to a limb or other suitable body area of the subject.

In some embodiments, the subject is a human. Alternatively, the subject is a non-human mammal.

In some embodiments, the volume of transdermal formulation that is applied to the skin in each dose is in the range of about 0.01 to 10 mL. In a preferred embodiment the volume of drug applied to the skin in each dose is in the range of about 0.1 to 5.0 mL and in a yet more particularly preferred embodiment is in the range of about 0.2 to 2.0 mL.

In some embodiments, the formulation is applied to an area of skin of about 0.5, 1, 2, 5, 10, 20, 50, 100, 140 or 200 cm² or any area between these numbers.

The compositions of the invention are suitable for use on mammalian skin.

In some embodiments, the compositions of the invention are suitable for acute or temporary use.

### Packaging

Compositions of the present disclosure may, if desired, be presented in a sealed pouch, each pouch containing a single dose of the inventive formulation. More than one sealed pouch may, if desired, be packaged to provide, in one package, multiple individual doses of the inventive formulation.

### Devices and Kits

In some embodiments, the topical formulations are prepared as part of a device suitable for providing rapid local anesthesia. In some embodiments, the device comprises an annulus of pressure sensitive adhesive, within the inner circumference of which is provided any one of the topical formulations disclosed herein. In some embodiments, in lieu of the annulus, the pressure sensitive adhesive may be any other shape to cover the area to be affected. In some embodiments, the device further comprises a backing film on which the topical formulation is applied, where the backing film covers the inner circumference of the annulus of pressure sensitive adhesive. In some embodiments, the backing film is intended to be removed after application of the device such that the inner circumference of the annulus of pressure sensitive adhesive is exposed. This exposure allows for subsequent actions, such as allowing access with a needle. In some embodiments, the topical formulation is provided as a single dose packet (e.g., a packet for a single use). In some embodiments, the backing film may be substituted for or used in conjunction with an occlusive, waterproof dressing to cover the affected area for the duration of anesthetization by the topical formulation.

Compositions provided for herein are, in some embodiments, sold or otherwise provided in the form of a kit. In some embodiments, the kit comprises a sealed sachet or pouch which holds the inventive formulation. Alternatively, the kit may comprise several sealed sachets or pouches, each holding the inventive formulation. In some embodiments, the sachet or pouch substantially protects the formulation from atmospheric oxygen until the sachet or pouch is opened and the inventive formulation applied to a subject. In some embodiments, the kit comprises one or more of the devices disclosed herein, which may comprise the topical formulation, pressure sensitive adhesive, and/or backing film or occlusive, waterproof dressing. In some embodiments are disclosed kits for use in anesthetizing a target area of skin in a subject. In some embodiments, the kits comprise a predetermined dose of a topical anesthetic, an annulus of pressure sensitive adhesive comprising an outer perimeter that adheres to the skin of the subject, and an open center region in which the skin is exposed. In some embodiments, the kits further comprises an occlusive, waterproof dressing or a backing film to cover the open center region and target area. In some embodiments, the topical anesthetic is any one of the topical formulations disclosed herein. In some embodiments, the topical anesthetic comprises lidocaine. In several embodiments, the topical anesthetic is present in the topical formulation in an amount about 4 % or less than about 4%. In some embodiments, the lidocaine is optionally in gel format. In some embodiments, the annulus of pressure sensitive adhesive comprises a silicone ring. In some embodiments, the predetermined dose of the topical anesthetic is provided as a single dose/use packet.

In some embodiments, the kit additionally comprises a notice. The notice may be in a form approved by a governmental agency regulating the manufacture, use, or sale of pharmaceuticals, the notice indicating approval by the agency. In one aspect the notice may contain information about how to safely apply the formulation. The notice may include information concerning the identity of the active ingredients in the formulation. In one embodiment the active ingredient listed on the notice comprises or consists of lidocaine.

The invention is generally disclosed herein using affirmative language to describe the numerous embodiments. The invention also includes embodiments in which subject matter is excluded, in full or in part, such as substances or materials, method steps and conditions, protocols, or procedures.

The following non-limiting examples are illustrative of the present application:

### EXAMPLES

Some of the embodiments discussed above are elaborated upon in further detail in the following examples, which are not in any way intended to limit the scope of the present disclosure. Those in the art will appreciate that many other embodiments that not be specifically discussed in the examples are also provided for herein as described above and in the claims.

### Example 1. Formulation Preparation and Assessment

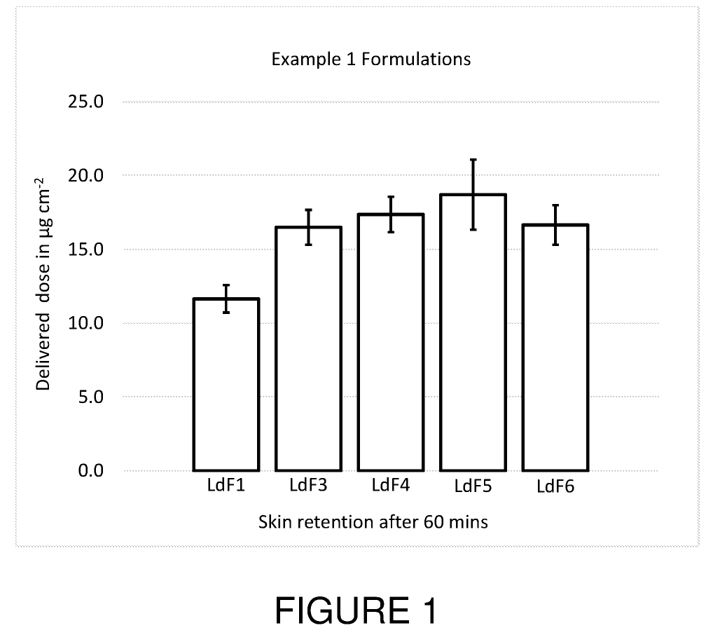

Formulations of compositions as provided in Table 1 were prepared using the general procedure described under Example 21. The amounts of lidocaine that have permeated into and which are retained within porcine skin (the combined epidermal and dermal tissue) at 60 mins following formulation application as measured by extraction from the skin according to the procedure described under Example 22 are provided in FIG. 1, expressed in µg per cm² of application area.

The concentration of acrylate pressure-sensitive adhesive in Table 1 is provided on a ‘wet’ basis and the solids content of the adhesives is, on average, 42%, so that after dry-down, the lidocaine concentration in the films used in this skin delivery study is about 9.5% w/w. FIG. 1 illustrates that the intrinsic extent of lidocaine delivery from a simple patch formulation is relatively low and it varies little as the nature of the acrylate polymer system is changed. The embodiments in the subsequent examples demonstrate compositions comprising lidocaine that improve topical bioavailability and efficacy.

TABLE 1

Lidocaine formulation compositions. All amounts are given in % w/w

| | LdF1 | LdF3 | LdF4 | LdF5 | LdF6 |
|---|---|---|---|---|---|
| Lidocaine | 4 | 4 | 4 | 4 | 4 |
| DURO-TAK UN1133 | 96 | | | | |
| DURO-TAK 387-2516 | | 96 | | | |
| DURO-TAK 87-4098 | | | 96 | | |
| DURO-TAK 387-2054 | | | | 96 | |
| GELVA GMS 7883 | | | | | 96 |

### Example 2. Formulation Preparation and Assessment

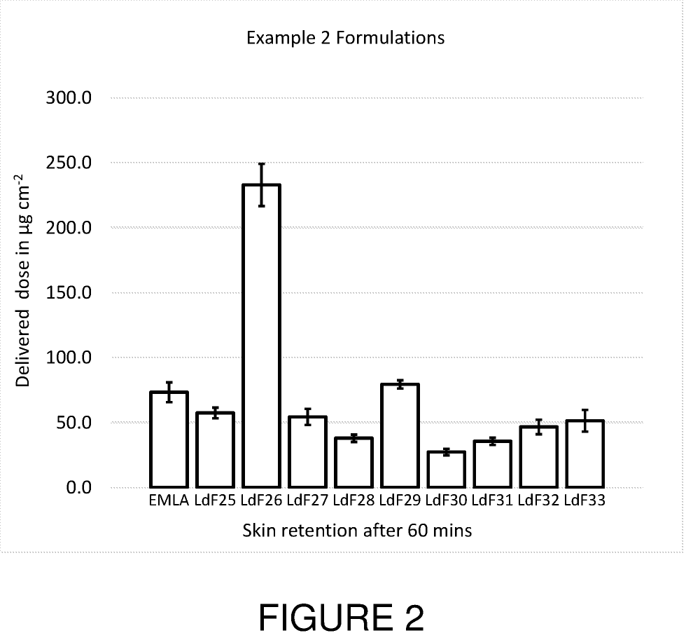

Formulations of compositions as provided in Table 2 were prepared using the general procedure described under Example 21. The amounts of lidocaine that have permeated into and which are retained within porcine skin (the combined epidermal and dermal tissue) at 60 mins following formulation application as measured by extraction from the skin according to the procedure described under Example 22 are provided (referenced to EMLA® Cream (lidocaine 2.5% and prilocaine 2.5%)) in FIG. 2, expressed in µg per cm² of application area.

It is frequently stated in the art that a solution of an active agent in pure DMSO will provide the highest possible levels of dermal delivery and transdermal permeation. FIG. 2 illustrates that a solution of lidocaine in DMSO is less effective at delivering lidocaine into porcine skin over 60 minutes than EMLA®, which has only 2.5% w/w of lidocaine. More surprisingly, the introduction of D-limonene provides a substantial increase in delivery, even though other molecular accelerants and MPEs have little effect in the same system. FIG. 2 shows that composition LdF26, composed of 4% lidocaine, 86% DMSO, and 10% limonene exhibit significantly enhanced (about 233 µg/cm²) delivery of lidocaine, relative to the LdF25 DMSO and EMLA controls. Composition LdF29, which contains the surfactant Brij L4, also exhibits a modest advantage over control formulations.

TABLE 2

Lidocaine formulation compositions. All amounts are given in % w/w

| LdF25 | LdF25 | LdF26 | LdF27 | LdF28 | LdF29 | LdF30 | LdF31 | LdF32 | LdF33 |
|---|---|---|---|---|---|---|---|---|---|
| Lidocaine | 4 | 4 | 4 | 4 | 4 | 4 | 4 | 4 | 4 |
| DMSO | 96 | 86 | 86 | 86 | 86 | 86 | 86 | 86 | 86 |
| Limonene | | 10 | | | | | | | |
| Ethanol | | | 10 | | | | | | |
| Transcutol | | | | 10 | | | | | |
| Brij L4 | | | | | 10 | | | | |
| Levulinic acid | | | | | | 10 | | | |
| Propylene glycol | | | | | | | 10 | | |
| Dimethyl isosorbide | | | | | | | | 10 | |
| Benzyl alcohol | | | | | | | | | 10 |

### Example 3. Formulation Preparation and Assessment

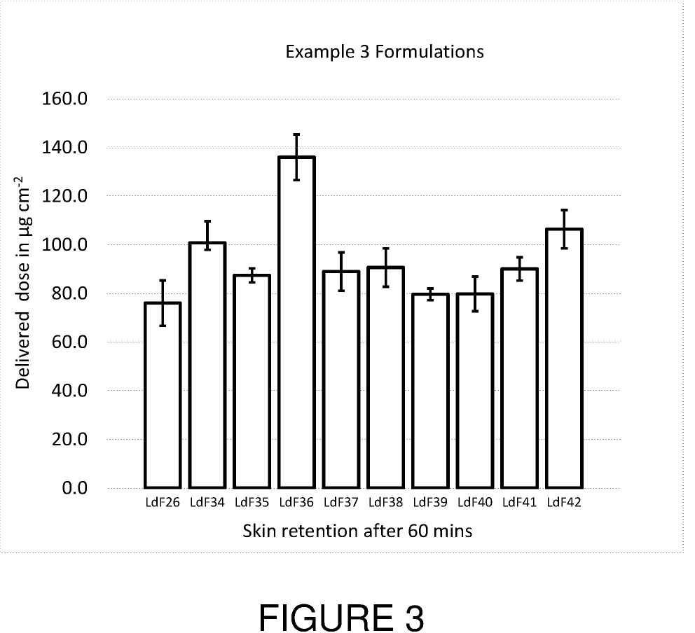

Formulations of compositions as provided in Table 3 were prepared using the general procedure described under Example 21. The amounts of lidocaine that have permeated into and which are retained within porcine skin (the combined epidermal and dermal tissue) at 60 mins following formulation application as measured by extraction from the skin according to the procedure described under Example 22 are provided in FIG. 3, expressed in µg per cm² of application area.

FIG. 3 illustrates that several of the MPEs known in the prior art have little effect on the delivery of lidocaine into the skin over 60 minutes. While the overall delivery numbers are lower than in FIG. 2 (reflecting the donor to donor variability of natural animal skin), formulation LdF36 comprising 10% w/w of isopropyl palmitate and formulation LdF42 comprising 10% w/w of isopropyl myristate both evidence enhanced lidocaine delivery levels as compared to LdF26. These results show that, in accordance with several embodiments disclosed herein, addition of fatty acid esters improve topical delivery of lidocaine compositions, with isopropyl palmitate and isopropyl myristate (isopropyl esters of a C14-C18 fatty acid) having the greatest effect under the conditions tested.

TABLE 3

Lidocaine formulation compositions. All amounts are given in % w/w

| | LdF26 | LdF34 | LdF35 | LdF36 | LdF37 | LdF38 | LdF39 | LdF40 | LdF41 | LdF42 |
|---|---|---|---|---|---|---|---|---|---|---|
| Lidocaine | 4 | 4 | 4 | 4 | 4 | 4 | 4 | 4 | 4 | 4 |
| DMSO | 86 | 61 | 56 | 45 | 45 | 45 | 45 | 45 | 45 | 45 |
| Ethanol | | 20 | 20 | 31 | 31 | 31 | 31 | 31 | 31 | 31 |
| Limonene | 10 | 10 | 10 | 10 | 10 | 10 | 10 | 10 | 10 | 10 |
| Menthol | | 5 | | | | | | | | |
| a-Terpineol | | | 10 | | | | | | | |
| Isopropyl palmitate | | | | 10 | | | | | | |
| Hexylene glycol | | | | | 10 | | | | | |
| Diisopropyl adipate | | | | | | 10 | | | | |
| Glycerol monooleate | | | | | | | 10 | | | |
| Diethyl sebacate | | | | | | | | 10 | | |
| Octyldodecanol | | | | | | | | | 10 | |
| Isopropyl myristate | | | | | | | | | | 10 |

### Example 4. Formulation Preparation and Assessment

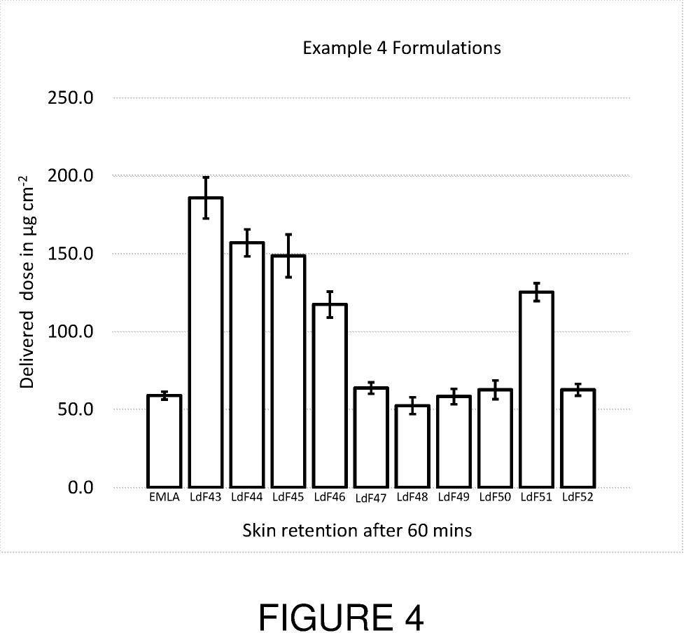

Formulations of compositions as provided in Table 4 were prepared using the general procedure described under Example 21. The amounts of lidocaine that have permeated into and which are retained within porcine skin (the combined epidermal and dermal tissue) at 60 mins following formulation application as measured by extraction from the skin according to the procedure described under Example 22 are provided (referenced to EMLA® Cream (lidocaine 2.5% and prilocaine 2.5%)) in FIG. 4, expressed in µg per cm² of application area.

FIG. 4 further illustrates that the effect of a given MPE that is known in the prior art on the delivery of lidocaine into the skin over 60 minutes, that is its effectiveness as a molecular accelerant for lidocaine, can vary broadly from that of another MPE, even those that are nominally similar. Amongst the MPEs considered in Table 4, dipropylene glycol, PEG-7 methyl ether, lauryl lactate and oleyl oleate evidence some role as molecular pentrations, but are inferior (in this study) to D-limonene.

TABLE 4

Lidocaine formulation compositions. All amounts are given in % w/w

| | LdF43 | LdF44 | LdF45 | LdF46 | LdF47 | LdF48 | LdF49 | LdF50 | LdF51 | LdF52 |
|---|---|---|---|---|---|---|---|---|---|---|
| Lidocaine HCL | 4 | 4 | 4 | 4 | 4 | 4 | 4 | 4 | 4 | 4 |
| DMSO | 66 | 56 | 56 | 52 | 45 | 45 | 45 | 45 | 45 | 45 |
| Ethanol | 20 | 20 | 20 | 31 | 31 | 31 | 31 | 31 | 31 | 31 |
| Limonene | 10 | 10 | 10 | 10 | 10 | 10 | 10 | 10 | 10 | 10 |
| Dipropylene glycol | | 10 | | | | | | | | |
| PEG-7 methyl ether | | | 10 | | | | | | | |
| Lauryl lactate | | | | 3 | | | | | | |
| Lauric diethanolamide | | | | | 10 | | | | | |
| Oleic acid | | | | | | 10 | | | | |
| Octyldodecanol | | | | | | | 10 | | | |
| Ceraphyl 41 | | | | | | | | 10 | | |
| Oleyl oleate | | | | | | | | | 10 | |
| Capric/caprylic triglyceride | | | | | | | | | | 10 |

### Example 5. Formulation Preparation and Assessment

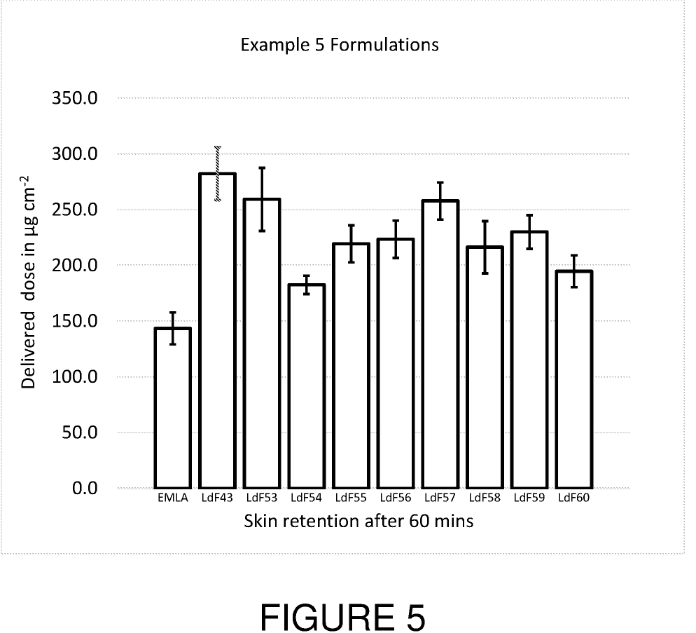

Formulations of compositions as provided in Table 5 were prepared using the general procedure described under Example 21. The amounts of lidocaine that have permeated into and which are retained within porcine skin (the combined epidermal and dermal tissue) at 60 mins following formulation application as measured by extraction from the skin according to the procedure described under Example 22 are provided (referenced to EMLA® Cream (lidocaine 2.5% and prilocaine 2.5%)) in FIG. 5, expressed in µg per cm² of application area.

FIG. 5 further illustrates that several MPEs that are known in the prior art have little incremental effect on the delivery of lidocaine into the skin over 60 minutes, relative to the simpler formulation comprising DMSO, ethanol and to D-limonene.

TABLE 5

Lidocaine formulation compositions. All amounts are given in % w/w

| | LdF43 | LdF53 | LdF54 | LdF55 | LdF56 | LdF57 | LdF58 | LdF59 | LdF60 |
|---|---|---|---|---|---|---|---|---|---|
| Lidocaine | 4 | 4 | 4 | 4 | 4 | 4 | 4 | 4 | 4 |
| DMSO | 66 | 61 | 61 | 61 | 61 | 61 | 61 | 61 | 61 |
| Ethanol | 20 | 20 | 20 | 20 | 20 | 20 | 20 | 20 | 20 |
| Limonene | 10 | 10 | 10 | 10 | 10 | 10 | 10 | 10 | 10 |
| Cyclomethicone | | 5 | | | | | | | |
| PPG 15 stearyl ether | | | 5 | | | | | | |
| Span 20 (sorbitan monolaurate) | | | | 5 | | | | | |
| Brij L 23 69 LQ (Laureth-23) | | | | | 5 | | | | |
| Brij S 20 So MH (Steareth-21) | | | | | | 5 | | | |
| Cocamide DEA | | | | | | | 5 | | |
| Polysorbate 80 | | | | | | | | 5 | |
| Ethyl oleate | | | | | | | | | 5 |

### Example 6. Formulation Preparation and Assessment

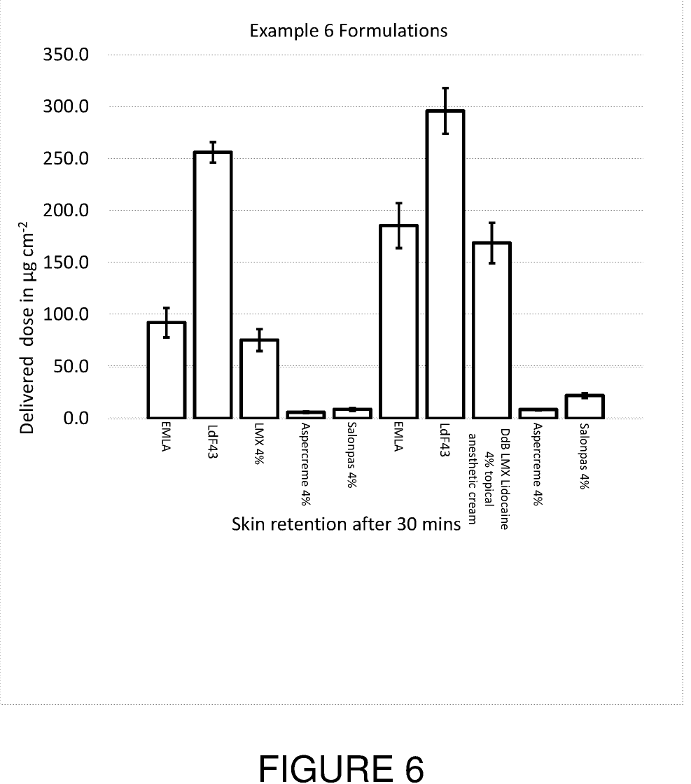

Formulation LdF43 (Table 5) was prepared using the general procedure described under Example 21. The amounts of lidocaine that have permeated into and which are retained within porcine skin at 30 mins following formulation application from LdF43, referenced to EMLA® Cream (lidocaine 2.5% and prilocaine 2.5%), LMX Lidocaine 4% topical anesthetic cream, Aspercreme Lidocaine Patch 4%, and Salonpas Lidocaine 4% Pain relieving gel patch (measured according to the procedure described under Example 22) are provided in FIG. 6, expressed in µg per cm² of application area.

The data presented in FIG. 6 illustrates the superiority of several embodiments of the formulations disclosed herein (using LdF43 as a non-limiting example) relative to commercial lidocaine formulations with respect to providing active agent delivery into skin.

### Example 7. Formulation Preparation and Assessment

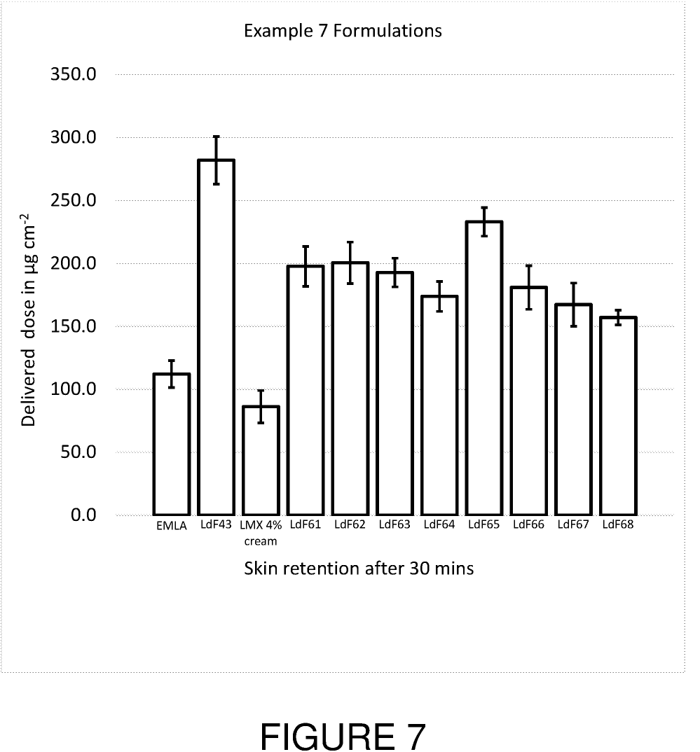

Formulations of compositions as provided in Table 6 were prepared using the general procedure described under Example 21. The amounts of lidocaine that have permeated into and which are retained within porcine skin (the combined epidermal and dermal tissue) at 30 mins following formulation application as measured by extraction from the skin according to the procedure described under Example 22 are provided (referenced to EMLA® Cream and LMX Lidocaine 4% topical anesthetic cream) in FIG. 7, expressed in µg per cm² of application area.

FIG. 7 further illustrates the superiority of several embodiments of the formulations disclosed herein (using LdF43 as a non-limiting example) relative to commercial lidocaine formulations with respect to providing active agent delivery into skin. This data also shows that, surprisingly, in accordance with several embodiment, incorporation of additional individual excipients does not further enhance such delivery.

TABLE 6

Lidocaine formulation compositions All amounts are given in % w/w

| | LdF43 | LdF61 | LdF62 | LdF63 | LdF64 | LdF65 | LdF66 | LdF67 | LdF68 |
|---|---|---|---|---|---|---|---|---|---|
| Lidocaine | 4 | 4 | 4 | 4 | 4 | 4 | 4 | 4 | 4 |
| DMSO | 66 | 56 | 56 | 61 | 61 | 56 | 56 | 59 | 56 |
| Ethanol | 20 | 20 | 20 | 20 | 20 | 20 | 20 | 20 | 20 |
| Limonene | 10 | 10 | 10 | 10 | 10 | 10 | 10 | 10 | 10 |
| Ethanol | | 10 | | | | | | | |
| Transcutol | | | 10 | | | | | | |
| Brij L4 LQ (Laureth-4) | | | | 5 | | | | | |
| Levulinic acid | | | | | 5 | | | | |
| Propylene glycol | | | | | | 10 | | | |
| Dimethyl isosorbide (DMI) | | | | | | | 10 | | |
| Isopropyl palmitate | | | | | | | | 7 | |
| Hexylene glycol | | | | | | | | | 10 |

### Example 8. Formulation Preparation and Assessment

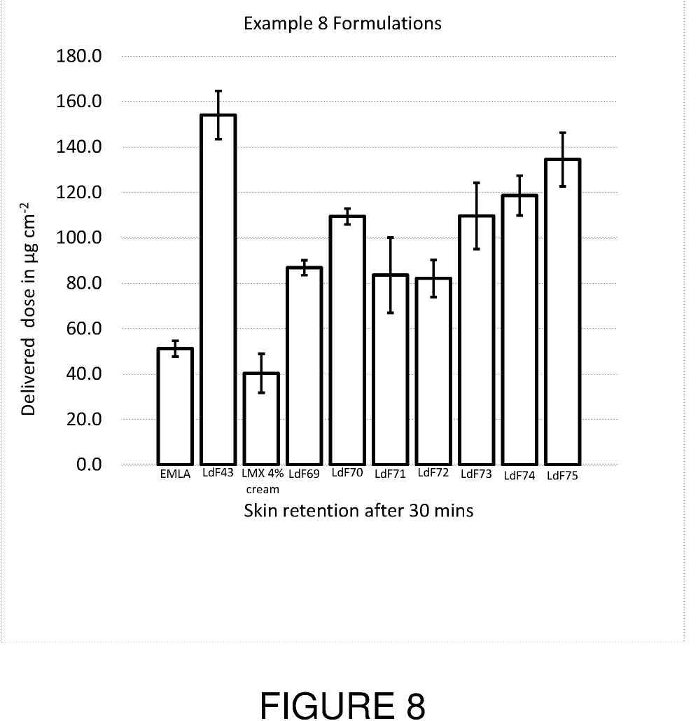

Formulations of compositions as provided in Table 7 were prepared using the general procedure described under Example 21. The amounts of lidocaine that have permeated into and which are retained within porcine skin (the combined epidermal and dermal tissue) at 30 mins following formulation application as measured by extraction from the skin according to the procedure described under Example 22 are provided (referenced to EMLA® Cream and LMX Lidocaine 4% topical anesthetic cream) in FIG. 8, expressed in µg per cm² of application area.

FIG. 8 further illustrates the superiority of several embodiments of the formulations disclosed herein (using LdF43 as a non-limiting example) relative to commercial lidocaine formulations with respect to providing active agent delivery into skin. This data also shows that, surprisingly, in accordance with several embodiment, incorporation of additional individual excipients does not further enhance such delivery.

TABLE 7

Lidocaine formulation compositions. All amounts are given in % w/w

| | LdF43 | LdF69 | LdF70 | LdF71 | LdF72 | LdF73 | LdF74 | LdF75 |
|---|---|---|---|---|---|---|---|---|
| Lidocaine | 4 | 4 | 4 | 4 | 4 | 4 | 4 | 4 |
| DMSO | 66 | 56 | 63 | 56 | 56 | 61 | 56 | 61 |
| Ethanol | 20 | 20 | 20 | 20 | 20 | 20 | 20 | 20 |
| Limonene | 10 | 10 | 10 | 10 | 10 | 10 | 10 | 10 |
| Diisopropyl adipate | | 10 | | | | | | |
| Glycerol monooleate | | | 4 | | | | | |
| Diethyl sebacate | | | | 10 | | | | |
| Isopropyl myristate | | | | | 10 | | | |
| Polysorbate 20 | | | | | | 5 | | |
| Glycerin | | | | | | | 10 | |
| Propylene Carbonate | | | | | | | | 5 |

### Example 9. Formulation Preparation and Assessment

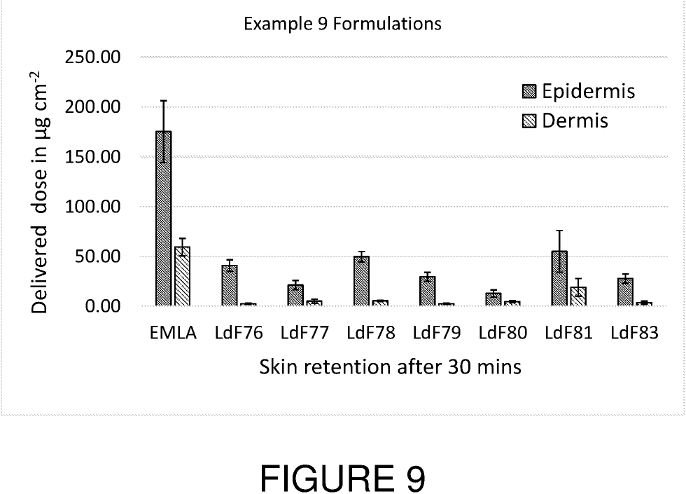

Formulations of compositions as provided in Table 8 were prepared using the general procedure described under Example 21. The amounts of lidocaine that have permeated into and which are retained within the epidermal and dermal compartments of human cadaver at 30 mins following formulation application as measured according to the procedure described under Example 22 are provided (referenced to EMLA® Cream) in FIG. 9, expressed in µg per cm² of application area.

FIG. 9 illustrates that selected patch formulation compositions do not provide enhanced level of lidocaine delivery into the epidermal and dermal compartments of human cadaver over a 30 minute period relative to a commercial lidocaine cream formulation.

TABLE 8

Lidocaine formulation compositions. All amounts are given in % w/w

| | LdF76 | LdF77 | LdF78 | LdF79 | LdF80 | LdF81 | LdF83 |
|---|---|---|---|---|---|---|---|
| Lidocaine | 4 | 4 | 4 | 4 | 4 | 4 | 4 |
| Gelva GMS 788 | 96 | 86 | 76 | 66 | 56 | 46 | 56 |
| Limonene | | 10 | 20 | 30 | 40 | 50 | 20 |
| DMSO | | | | | | | 20 |

### Example 10. Formulation Preparation and Assessment

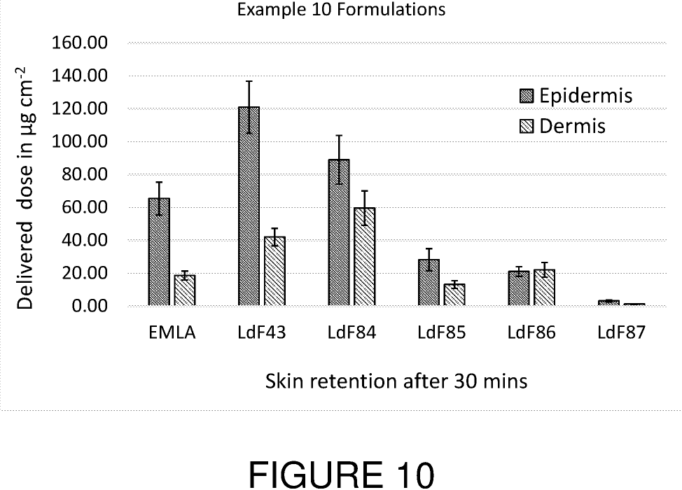

Formulations of compositions as provided in Table 9 were prepared using the general procedure described under Example 21. The amounts of lidocaine that have permeated into and which are retained within the epidermal and dermal compartments of human cadaver at 30 mins following formulation application as measured according to the procedure described under Example 22 are provided (referenced to EMLA® Cream) in FIG. 10, expressed in µg per cm² of application area.

FIG. 10 illustrates the superiority of the several embodiments of the formulations disclosed herein, with LdF43 (and a modification, LdF84, that has altered proportions of DMSO and ethanol) as non-limiting examples, at providing enhanced active agent delivery into the epidermal and dermal compartments of human skin.

TABLE 9

Lidocaine formulation compositions. All amounts are given in % w/w

| | LdF43 | LdF84 | LdF85 | LdF86 | LdF87 |
|---|---|---|---|---|---|
| Lidocaine | 4 | 4 | 4 | 4 | 4 |
| DMSO | 66 | 45 | 16 | 6 | |
| Ethanol | 20 | 41 | 50 | 50 | 36 |
| Limonene | 10 | 10 | 10 | 10 | 20 |
| Eudragit S 100 | | | 20 | 20 | 30 |
| Propylene glycol | | | | 10 | 10 |

### Example 11. Formulation Preparation and Assessment

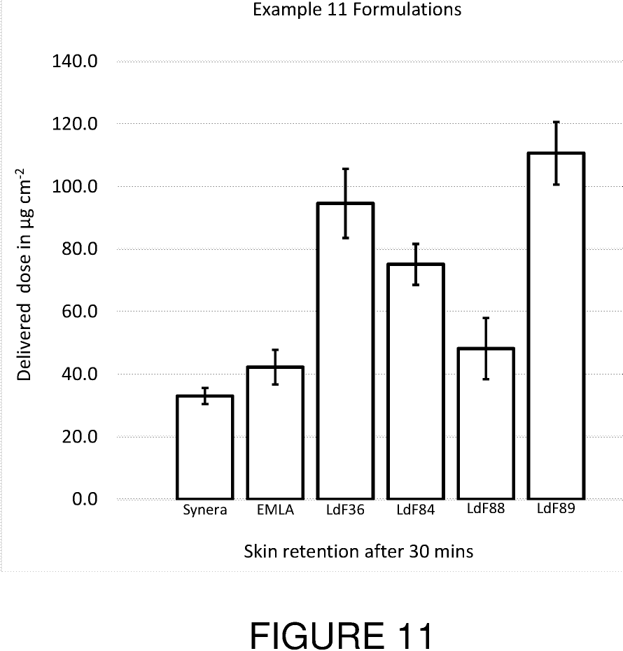

Formulations of compositions as provided in Table 10 were prepared using the general procedure described under Example 21. The amounts of lidocaine that have permeated into and which are retained within human cadaver skin (the combined epidermal and dermal tissue) at 30 mins following formulation application as measured according to the procedure described under Example 22 are provided (referenced to SYNERA® (lidocaine and tetracaine) Topical Patch and EMLA® Cream) in FIG. 11, expressed in µg per cm² of application area.

FIG. 11 illustrates the superiority of several embodiments of the formulations disclosed herein, with LdF84 (and a modification, LdF89, that has higher viscosity following incorporation of hydroxypropyl cellulose (“HPC”)) as non-limiting examples, at providing enhanced active agent delivery into human skin.

TABLE 10

Lidocaine formulation compositions. All amounts are given in % w/w

| | | | | |
|---|---|---|---|---|
| | LdF36 | LdF84 | LdF88 | LdF89 |
| Lidocaine | 4 | 4 | 2 | 4 |
| DMSO | 45 | 45 | 45 | 45 |
| Ethanol | 34 | 41 | 36 | 32 |
| Limonene | 10 | 10 | 10 | 10 |
| Isopropyl palmitate | 7 | | 7 | 7 |
| HPC | | | | 2 |
| Isopropyl myristate | | | | |

### Example 12. Formulation Preparation and Assessment

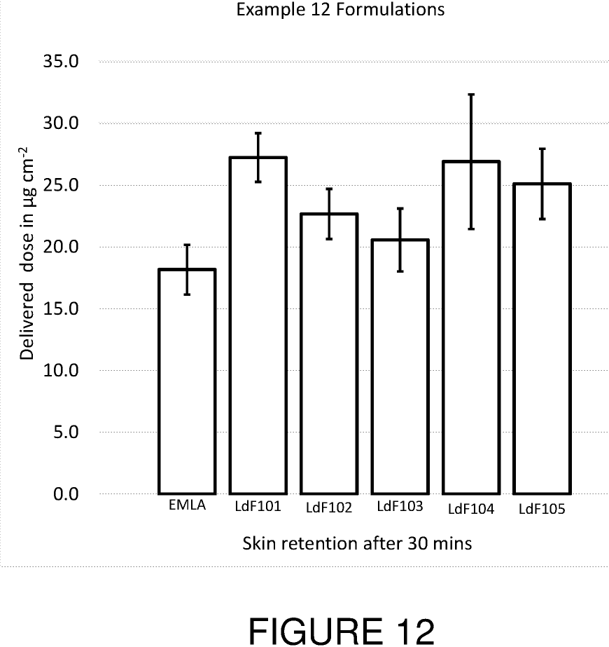

Formulations of compositions as provided in Table 11 were prepared using the general procedure described under Example 21. The amounts of lidocaine that have permeated into and which are retained within human cadaver skin (the combined epidermal and dermal tissue) at 30 mins following formulation application as measured according to the procedure described under Example 22 are provided (referenced to EMLA® Cream) in FIG. 12, expressed in µg per cm² of application area.

FIG. 12 illustrates that increased concentration of HPC leads to enhanced levels of active agent delivery into human skin (over a 30 minute period).

TABLE 11

Lidocaine formulation compositions. All amounts are given in % w/w

| | LdF101 | LdF102 | LdF103 | LdF104 | LdF105 |
|---|---|---|---|---|---|
| Lidocaine | 4 | 4 | 4 | 4 | 4 |
| DMSO | 34 | 34 | 34 | 44 | 34 |
| Ethanol | 32 | 32 | 37 | 37 | 37 |
| Limonene | 10 | 10 | 5 | 5 | 5 |
| Isopropyl palmitate | 7 | 7 | 7 | 7 | 7 |
| Propylene glycol | 10 | | | | 10 |
| HPC | 3 | 3 | 3 | 3 | 3 |
| Croda GTCC | | 10 | 10 | | |

### Example 13. Formulation Preparation and in Vivo Assessment

Formulations of compositions as provided in Table 12 were prepared using the general procedure described under Example 21. Each was evaluated in vivo for numbing potential and irritation potential according to the procedure described under Example 23. The results here indicate that, according to several embodiments, excipients such as isopropyl myristate and isopropyl palmitate used in compositions with high transdermal permeation do not cause undue irritation.

TABLE 12

Lidocaine formulation compositions. All amounts are given in % w/w

| | LdF106 | LdF107 | LdF108 | LdF109 | LdF110 | LdF111 | LdF112 | LdF113 | LdF114 | LdF115 | LdF116 | LdF117 | LdF118 | LdF119 | LdF120 |
|---|---|---|---|---|---|---|---|---|---|---|---|---|---|---|---|
| Lidocaine | 4 | 4 | 4 | 4 | 4 | 4 | 4 | 4 | 4 | 4 | 4 | 4 | 4 | 4 | 4 |
| DMSO | | | 18 | 16 | | | | | | | | | | | |
| Ethanol | 50 | 50 | 50 | 50 | 50 | 50 | 50 | | | | | | | | |
| Limonene | 10 | 10 | 10 | 10 | 10 | 10 | 10 | 10 | 10 | 10 | 10 | 10 | 10 | 10 | 10 |
| Isopropyl palmitate | 7 | 7 | 7 | 7 | 7 | 7 | 7 | 7 | 7 | 7 | 7 | 7 | 7 | 7 | 7 |
| Brij L4 | 5 | 5 | 5 | 5 | 5 | 5 | 5 | | | 5 | 5 | 5 | 5 | 5 | 5 |
| Isopropyl myristate | 21 | | | | | | | 26 | 16 | 11 | 11 | 11 | 7 | 7 | 7 |
| Propylene glycol | | 21 | | | | | | | | | | | | | |
| Lauryl lactate | | | 3 | | | | | | | | | | | | |
| Cyclomethicone | | | | 5 | | | | | | | | | | | |
| Peg-7 methyl ether | | | | | 21 | | | | 10 | 10 | | | 7 | 7 | |
| Isostearic acid | | | | | | 21 | | | | | 10 | | 7 | | 7 |
| Isostearyl alcohol | | | | | | | 21 | | | | | 10 | | 7 | 7 |
| HPC | 3 | 3 | 3 | 3 | 3 | 3 | 3 | 3 | 3 | 3 | 3 | 3 | 3 | 3 | 3 |
| Ethanol | | | | | | | | 50 | 50 | 50 | 50 | 50 | 50 | 50 | 50 |
| Pain Score | 6 | 10 | | | 4 | 3 | 4 | 8 | 7 | 5 | 8 | 6 | 6 | 7 | 7 |
| Irritancy Score | 0 | 1 | 7 | 7 | 1 | 1 | 1 | 0 | 0 | 0 | 0 | 0 | 0 | 0 | 0 |

### Example 14. Formulation Preparation and in Vivo Assessment

Formulations of compositions as provided in Table 13 were prepared using the general procedure described under Example 21. Each was evaluated in vivo for numbing potential and irritation potential according to the procedure described under Example 23.

TABLE 13

Lidocaine formulation compositions. All amounts are given in % w/w

| | LdF146 | LdF147 | LdF148 | LdF149 | LdF150 | LdF151 | LdF152 | LdF153 | LdF154 | LdF155 | LdF156 | LdF157 | LdF158 |
|---|---|---|---|---|---|---|---|---|---|---|---|---|---|
| Lidocaine | 4 | 4 | 4 | 4 | 4 | 4 | 4 | 4 | 4 | 4 | 4 | 4 | 4 |
| Gelva GMS 788 | 41 | 44 | 44 | 44 | 42 | 42 | 40 | | | | | | |
| DURO-TAK 87-4098 | 41 | 44 | 44 | 44 | 42 | 42 | 40 | 89 | 89 | 89 | 85 | 85 | 81 |
| DMSO | 5 | | | | | | | | | | | | |
| Diethyl sebacate | 5 | | | | | | | | | | | | |
| Levulinic acid | 3 | 3 | 3 | 3 | 3 | 3 | 3 | 1.5 | 1.5 | 1.5 | 1.5 | 1.5 | 1.5 |
| Brij L4 LQ | 1 | 2 | 2 | 2 | 2 | 2 | 2 | 1.5 | 1.5 | 1.5 | 1.5 | 1.5 | 1.5 |
| Isopropyl palmitate | | 4 | | | 4 | 4 | 4 | 4 | | | 4 | 4 | 4 |
| Isopropyl myristate | | | 4 | | 4 | | 4 | | 4 | | 4 | | 4 |
| Isostearyl alcohol | | | | 4 | | 4 | 4 | | | 4 | | 4 | 4 |
| Eudragit S100 | | | | | | | | | | | | | |
| Ethanol | | | | | | | | | | | | | |
| Pain Score | 2 | 2 | 2 | 2 | 2 | 2 | 2 | 2 | 2 | 2 | 2 | 2 | 2 |

| | LdF159 | LdF160 | LdF161 | LdF162 | LdF163 | LdF164 | LdF166 | LdF167 | LdF173 |
|---|---|---|---|---|---|---|---|---|---|
| Lidocaine | 4 | 4 | 4 | 4 | 4 | 4 | 4 | 4 | 2 |
| Gelva GMS 788 | | | | | | | | | |
| DURO-TAK 87-4098 | 87 | 87 | 87 | 83 | 83 | 79 | 81 | | 85 |
| DMSO | | | | | | | | | |
| Diethyl sebacate | | | | | | | | | |
| Levulinic acid | 3 | 3 | 3 | 3 | 3 | 3 | 1.5 | 1.5 | 3 |
| Brij L4 LQ | 2 | 2 | 2 | 2 | 2 | 2 | 1.5 | 1.5 | 2 |
| Isopropyl palmitate | 4 | | | 4 | 4 | 4 | 4 | 4 | 4 |
| Isopropyl myristate | | 4 | | 4 | | 4 | 4 | 4 | 4 |
| Isostearyl alcohol | | | 4 | | 4 | 4 | 4 | 4 | |
| Eudragit S100 | | | | | | | | 12 | |
| Ethanol | | | | | | | | 69 | |
| Pain Score | 2 | 2 | 2 | 2 | 2 | 2 | 2 | 2 | 2 |

### Example 15. Formulation Preparation and in Vivo Assessment

Formulations of compositions as provided in Table 14 were prepared using the general procedure described under Example 21. Each was evaluated in vivo for numbing potential and irritation potential according to the procedure described under Example 23.

TABLE 14

Lidocaine formulation compositions. All composition amounts are given in % w/w

| | LdF165 | LdF180 | LdF181 | LdF182 | LdF183 | LdF184 | LdF185 | LdF186 | LdF187 | LdF188 | LdF189 | LdF190 | LdF191 |
|---|---|---|---|---|---|---|---|---|---|---|---|---|---|
| Lidocaine | 4 | 4 | 4 | 4 | 4 | 4 | 4 | 4 | 4 | 4 | 4 | 4 | 4 |
| Ethanol | 51 | 40 | 40 | 40 | 40 | 40 | 40 | 46 | 39 | 39 | 46 | 46 | 39 |
| Limonene | 10 | 10 | 10 | 10 | 10 | 10 | 10 | 10 | 10 | 10 | 10 | 10 | 10 |
| Isopropyl palmitate | 7 | 7 | 7 | 7 | 7 | 7 | 7 | 7 | 7 | 7 | 7 | 7 | 7 |
| Brij L4 | 5 | 5 | 5 | 5 | 5 | 5 | 5 | 5 | 5 | 5 | 5 | 5 | 5 |
| Isopropyl myristate | 11 | 11 | 11 | 11 | 11 | 11 | 11 | 11 | 11 | 11 | 11 | 11 | 11 |
| Isostearyl alcohol | 10 | 10 | 10 | 10 | 10 | 10 | 10 | 10 | 10 | 10 | 10 | 10 | 10 |
| Klucel MF | 2 | | | | | | | 4 | 4 | 4 | 4 | 4 | 4 |
| Lauryl lactate | | 10 | | | | | | 3 | | | | | |
| Diisopropyl adipate | | | 10 | | | | | | 10 | | | | |
| Propylene glycol monolaurate | | | | 10 | | | | | | 10 | | | |
| Glyceryl monolinoleate | | | | | 10 | | | | | | | | |
| Levulinic acid | | | | | | 10 | | | | | | 3 | |
| Dimethyl isosorbide | | | | | | | 10 | | | | | | 10 |
| HPC HY119 | | 3 | 3 | 3 | 3 | 3 | 3 | | | | | | |
| Pain Score | 1 | 3 | 3 | 2 | 3 | 3 | 3 | 3 | 1 | 3 | 3 | 2 | 3 |

### Example 16. Formulation Preparation and in Vivo Assessment

Formulations of compositions as provided in Table 15 were prepared using the general procedure described under Example 21. Each was evaluated in vivo for numbing potential and irritation potential according to the procedure described under Example 23. The results here indicate that DMSO at low concentrations is useful as a permeating agent, as in accordance with several embodiments .

TABLE 15

Lidocaine formulation compositions. All compositions amounts are given in % w/w

| | LdF140 | LdF141 | LdF142 | LdF143 | LdF144 | LdF145 | LdF169 | LdF170 | LdF171 | LdF193 | LdF194 | LdF195 | LdF196 |
|---|---|---|---|---|---|---|---|---|---|---|---|---|---|
| Lidocaine | 4 | 4 | 4 | 4 | 4 | 4 | 4 | 4 | 4 | 4 | 4 | 4 | 4 |
| Ethanol | 70 | 40 | 72 | 69 | 64 | 64 | 86 | 72 | 80 | 35 | 35 | | |
| Eudragit RSPO | | | | | | | 10 | | | 15 | 15 | | |
| Eudragit NE 30D | | | | | | | | 10 | 16 | | | | |
| Eudragit S100 | 9 | 9 | 7 | 7 | 7 | 7 | | | | | | | |
| Limonene | 10 | 10 | 10 | 10 | 10 | 10 | | 10 | | | | 10 | |
| Isopropyl palmitate | 7 | 7 | 7 | 7 | 7 | 7 | | 1 | | | | | 7 |
| Brij L4 | | | | | | | | 1 | | | | | |
| Isopropyl myristate | | | | | | | | 1 | | | | | |
| Isostearyl alcohol | | | | | | | | 1 | | | | | |
| DMSO | | | | | 5 | | | | | | | 45 | 45 |
| Water | | | | | | | | | | 10 | 10 | | |
| Span 40 | | | | | | | | | | 2 | 2 | | |
| Urea | | | | | | | | | | 4 | | | |
| Transcutol | | 30 | | | | | | | | | | | |
| Glycerin | | | | | | | | | | | | 37 | 40 |
| Menthol | | | | | | | | | | | 4 | | |
| Ethyl acetate | | | | | | | | | | 30 | 30 | | |
| Transcutol | | | | | | 5 | | | | | | | |
| Klucel MF | | | | | | | | | | | | 4 | 4 |
| HPC | | | | 3 | 3 | 3 | | | | | | | |
| Pain Score | 3 | 2 | 3 | 3 | 1 | 3 | 3 | 3 | 3 | 4 | 4 | 4 | 4 |

### Example 17. Formulation Preparation and in Vivo Assessment

Formulations of compositions as provided in Table 16 were prepared using the general procedure described under Example 21. Each was evaluated in vivo for numbing potential and irritation potential according to the procedure described under Example 23. Compositions LdF123, LdF124, LdF125, LdF126, LdF128 and LdF129 evidence phase separation.

TABLE 16

Lidocaine formulation compositions. All composition amounts are given in % w/w

| | LdF121 | LdF122 | LdF123 | LdF124 | LdF125 | LdF126 | LdF127 | LdF128 | LdF129 |
|---|---|---|---|---|---|---|---|---|---|
| Lidocaine | 4 | 4 | 4 | 4 | 4 | 4 | 4 | 4 | 4 |
| Limonene | 10 | 10 | 10 | 10 | 10 | 10 | 10 | 10 | 10 |
| Isopropyl palmitate | 7 | 7 | 7 | 7 | 7 | 7 | 7 | 7 | 7 |
| Ethanol | 50 | 50 | 50 | 50 | 50 | 50 | 50 | | |
| Transcutol | 26 | | | | | 10 | 20 | 45 | 45 |
| Propylene glycol | | 26 | | 9 | 9 | 6 | 6 | 16 | 11 |
| Glycerin | | | 26 | | | | | | |
| Urea | | | | 5 | | | | 5 | 5 |
| Sodium lauryl sulphate | | | | | 5 | | | | 5 |
| Water | | | | 12 | 12 | 10 | | 10 | 10 |
| HPC | 3 | 3 | 3 | 3 | 3 | 3 | 3 | 3 | 3 |
| Pain Score | 3 | 3 | 3 | 3 | 3 | 3 | 3 | 3 | 3 |

### Example 18. Formulation Preparation and in Vivo Assessment

Formulations of compositions as provided in Table 17 were prepared using the general procedure described under Example 21. Each was evaluated in vivo for numbing potential and irritation potential according to the procedure described under Example 23.

TABLE 17

Lidocaine formulation compositions. All composition amounts are given in % w/w

| | LdF130 | LdF131 | LdF132 | LdF133 | LdF134 | LdF135 | LdF136 | LdF137 | LdF138 | LdF139 |
|---|---|---|---|---|---|---|---|---|---|---|
| Lidocaine | 4 | 4 | 4 | 4 | 4 | 4 | 4 | 4 | 4 | 4 |
| Limonene | 10 | 10 | 10 | 10 | 10 | 10 | 10 | 10 | 10 | 10 |
| Isopropyl palmitate | 7 | 7 | 7 | 7 | 7 | 7 | 7 | 7 | 7 | 7 |
| Ethanol | 40 | 40 | 40 | 40 | 40 | | | | | |
| Isostearic acid | | | | | | 29 | 24 | 19 | 14 | 9 |
| Transcutol | 34 | 29 | 24 | 19 | 14 | 45 | 45 | 45 | 45 | 45 |
| DMSO | 5 | 10 | 15 | 20 | 25 | 5 | 10 | 15 | 20 | 25 |
| Pain Score | 3 | 3 | 3 | 3 | 3 | 3 | 3 | 3 | 3 | 3 |

### Example 19. Formulation Preparation and in Vivo Assessment

Formulations of compositions as provided in Table 18 were prepared using the general procedure described under Example 21. Each was evaluated in vivo for numbing potential and irritation potential according to the procedure described under Example 23. Formulations LdF201, LdF202 and LdF203 are creams. The results here indicate that, in accordance with several embodiments disclosed herein, hexylene glycol enhances skin permeation. In several embodiments, this is accomplished with relatively nominal, to no, irritation at the application site as well.

TABLE 18

Lidocaine formulation compositions. All composition amounts are given in % w/w

| | LdF192 | LdF197 | LdF198 | LdF199 | LdF200 | LdF201 | LdF202 | LdF203 | LdF204 |
|---|---|---|---|---|---|---|---|---|---|
| Lidocaine | 4 | 4 | 4 | 4 | 4 | 4 | 4 | 4 | 4 |
| Limonene | 5 | 10 | | 10 | 10 | | 10 | 10 | |
| Isopropyl palmitate | 4 | | | | | | | | |
| Isopropyl myristate | 8 | | | | 15 | | | | |
| Isostearyl alcohol | 8 | | | | | | | | |
| DMSO | 10 | 45 | | 45 | 20 | | | | |
| Hexylene glycol | | 37 | | | | | | | |
| Glycerin | 5 | | | 37 | 30 | | | | |
| Diisopropyl adipate | 8 | | | | | | | | |
| Laureth-4 | 2.3 | | | | | | | | |
| Laureth-23 | 5.7 | | | | | | | | |
| Choline-generic acid | | | 92 | | | | | | |
| Gelot 64 | | | | | 11 | | | | |
| Schercemol GMIS ester | | | | | 5.3 | | | | |
| PEG-40 Stearate | | | | | 4.7 | 6 | 6 | 6 | 6 |
| Menthol | | | | | | 4 | 4 | 4 | |
| Cetostearyl alcohol | | | | | | 6 | 6 | | 10 |
| Water | 40 | | | | | 80 | 70 | 76 | |
| Water at pH 12 (NaOH) | | | | | | | | | 80 |
| HPC (HY119) | | 4 | 4 | 4 | | | | | |
| Pain Score | 4 | 1 | 5 | 5 | 3 | 3 | 3 | 3 | 3 |
| Irritancy Score | | 5 | 1 | 1 | 1 | 1 | 1 | 1 | 1 |

The formulation compositions disclosed in the above Examples demonstrate superior lidocaine delivery compared to commercially available products. Compositions comprising lidocaine, DMSO, ethanol, and limonene offer efficient transdermal permeation, while additional MPEs or excipients typically (but not always) cause a reduction in efficacy. Thus, in several embodiments, the compositions are MPE-free. In several embodiments, the compositions are excipient-free. Non-limiting examples of compositions according to embodiments provided for herein are: LdF43 (4% lidocaine, 66% DMSO, 20% ethanol, 10% limonene) and LdF84 (4% lidocaine, 45% DMSO, 41% ethanol, 10% limonene). Addition of at least one isopropyl ester of a C14-C18 fatty acid or HPC further improves permeation, in several embodiments, as seen with compositions LdF36 (4% lidocaine, 45% DMSO, 31% ethanol, 10% limonene, 10% isopropyl palmitate), LdF42 (4% lidocaine, 45% DMSO, 31% ethanol, 10% limonene, 10% isopropyl myristate), and LdF89 (4% lidocaine, 45% DMSO, 32% ethanol, 10% limonene, 7% isopropyl palmitate, 2% HPC).

### Example 20. Lidocaine Analytical Methods

A sample of lidocaine was obtained from Sigma (Catalog#: L7757). Assays of lidocaine in the various matrices generated in the examples were made using high performance liquid chromatography (“HPLC”) using ultraviolet (“UV”) detection at 220 nm or 360 nm using an Agilent 1100 system and an Agilent ZORBAX Eclipse Plus Phenyl Hexyl 5 µm, 4.6×100 mm column maintained at 40° C., with an Agilent ZORBAX Eclipse Plus 4.6×12.5 mm, 5 µm guard column. Mobile phase A comprised water with 0.1% phosphoric acid and mobile phase B comprised acetonitrile, at a flow rate of 1 mL min⁻¹. The gradient consisted of 5%, 5%, 95% and 95% mobile phase B at each of 0 min, 1 min, 6 min and 7 min with a post time of 2.5 min. Injection volumes were 5 or 10 µL. The calibration curve was developed using five standards with lidocaine concentrations ranging from 0.064 to 40 µg mL-1, using linear fitting force through zero.

### Example 21. General Procedure for Formulation Preparation

### (A) Solutions

The requisite amount of each excipient required for the given formulation composition is provided by accurate volumetric or gravimetric means, as appropriate, into a suitable container, such as a glass vial or media bottle. Appropriate solvents include but are not limited to water, oil, wax, fatty acids, lipids, detergents, nonpolar solvent, polar solvent, organic solvent, inorganic solvent, alcohol, ethanol, DMSO, isopropyl alcohol, propylene glycol, isopropyl myristate, polyethylene glycols, terpenes, limonene, esters, ethers, ketones, aldehydes, carboxylic acids, acids, bases or any combination or mixtures thereof. The requisite amount of lidocaine by accurate weighing is introduced. The suitable container is capped and the contents sonicated at room temperature until the lidocaine fully dissolves in the solvent.

### (B) Ointments and Gels

The requisite amount of each excipient required for the given formulation composition is provided by accurate volumetric or gravimetric means, as appropriate, into a suitable container, such as a glass or plastic vial. Excipients include but are not limited to solvents, emollients, stiffening agents, emulsifying agents, solubilizing agents, humectants, thickening agents, gelling agents, preservatives, permeation enhancers, chelating agents, antioxidants, acidifying agents, alkalizing agents, buffering agents, vehicles, carbomers, gums, xanthan gum, guar gum, carnauba wax, cetyl alcohol, cetyl ester wax, emulsifying wax, hydrous lanolin, lanolin, lanolin alcohols, microcrystalline wax, paraffin, petrolatum, polyethylene glycol, stearic acid, stearyl alcohol, white wax, yellow wax, wax absolute, polysorbate 20, polysorbate 80, polysorbate 60, poloxamer, sorbitan monostearate, sorbitan monooleate, sodium lauryl sulfate, ethers, esters, propylene glycol esters, propylene glycol ethers, diethylene glycol esters, diethylene glycol ethers, docusate, glycerin, propylene glycol, polyethylene glycol, sorbitol, methyl cellulose, carrageenan, colloidal silicon dioxide, gelatin, polyethylene oxide, alginic acid, alginate salts, sodium alginate, fumed silica, benzoic acid, propyl paraben, methyl paraben, imidurea, sorbic acid, sorbate salts, potassium sorbate, benzalkonium chloride, phenyl mercuric acetate, chlorobutanol, phenoxyethanol, ethanol, isopropyl alcohol, oleic acid, ethylenediaminetetraacetic acid (“EDTA”), butylated hydroxyanisole, butylated hydroxytoluene, citric acid, phosphoric acid, sodium hydroxide, sodium phosphate, triethanolamine, water, hexylene glycol, oleyl alcohol, propylene carbonate, or mineral oil, or any combination or mixtures thereof. The requisite amount of lidocaine by accurate weighing is introduced into a suitable container, such as a glass media bottle, with excipients. The contents are subjected to processes known in the art to form a semisolid preparation, such as a water-in-oil emulsion, oil-in-water emulsion, gel, colloid, homogenate, ointment, cream, lotion, suspension, foam, or shampoo.

### (C) Patches

Appropriate amounts of the liquid ingredients as taught herein are added to an amount of lidocaine in a container and appropriate amounts of the chosen acrylate copolymer solution or solutions are added. The container is covered or capped to prevent evaporative losses and the container contents are intimately mixed. Any air bubbles in the resulting homogeneous solution are removed, such as by a 10 minute period of sonication. A backing film (such as CoTran 9718 from 3 M (St. Paul, MN)) and bar applicator such as the bar film applicator from Mitutoyo, or a film applicator, such as from Byk-Gardner, are provided on a spreading platform. The solution is applied under the applicator and the applicator translated to produce a film of the uniform desired thickness on the backing membrane. The wet film is immediately placed horizontally for 30 minutes in an incubator or oven operating at 70° C. It is ensured that the incubator or oven is suitably vented to avoid exposure by the operators to the volatile solvents that are lost on drying of the film. The patch is removed from the incubator or oven and a suitable release liner, such as CoTran 9718 from 3M (St. Paul, MN), is applied to the sticky side of the patch.

A punch is used to produce a patch of the desired area and the patch is then heat-sealed in a suitable laminated pouch.

### Example 22. General Procedure for Skin Delivery Measurement

Different procedures were used in the skin delivery studies, depending on the format of the test formulation, the time period for the study, and on whether human or porcine skin was employed.

In first experiments with porcine skin, skin from 4 week-old pigs sourced from Thomas D. Morris, Inc. (Reisterstown, MD) was frozen following collection, shipped frozen over dry ice and stored frozen until used, when it is first allowed to thaw to room temperature. Pieces of skin some 1″ square are cut and placed on a paper towel soaked in phosphate-buffered saline solution at pH 7.4 (“PBS”). Semisolid test formulations (whether solution, cream, ointment) are dispensed onto the skin using a Nichiryo positive displacement pipette (Nichiryo America, Maryland Heights, MO) at a dose corresponding to some 9 mg per cm² of addressed skin area and spread over the addressed area using an instrument such as a blunt glass rod. At the end of the study period (30 or 60 minutes) excess formulation was wiped from the skin surface and the surface cleaned by applying 200 µL 50:50 water:ethanol, waiting 5 mins, and wiping dry with a Kimwipe. For prototype reservoir patch and matrix patch compositions the procedure is similar, with the patch (reservoir) or a 10 mm circular punch from a matrix patch formulation, typically some 200 µm in thickness, is applied to the skin surface. At the end of the 30 or 60 minute contact time, the patch is gently removed. After cleaning, the skin exterior is tapestripped either three or ten times, and the tapestrippings discarded. A punch of diameter in the range of 2 mm or about 2 mm to 1.5 cm or about 1.5 cm, such as 2 mm, 3 mm, 4 mm, 5 mm, 6 mm, 7 mm, 8 mm, 9 mm, 10 mm, 11 mm, 12 mm, 13 mm, 14 mm or 15 mm or about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm or about 15 mm, is taken of the central addressed area. The punched piece (epidermal and dermal sections are not separated) is placed into a 4 mL glass vial. 3 mL of DMSO (the “Extraction Fluid”) is added to the vial which is then maintained at 32° C. for 24 hours on an orbital shaker. At the end of the extraction period, aliquots of the Extraction Fluid are drawn and analyzed by the verified HPLC-UV method (Example 18).

In later experiments with human skin, Franz-type vertical diffusion cells (“FDC”s) were employed. Franz diffusion cells are a common and well-known method for measuring skin delivery and permeation. The general FDC procedure is described by Franz. In the present studies, FDCs with a 3.3 mL receptor well volume are used with split thickness human cadaver skin (0.015″-0.018″, obtained from AlloSource (Centennial, CO), Skin Bank NY Firefighters (New York, NY), Science Care (Phoenix, AZ), Allosource (Centennial, CO) or BioIVT (Westbury, NY). Skin is frozen following collection, shipped frozen over dry ice and stored frozen until used, when it is first allowed to thaw to room temperature. The FDC donor well addresses a skin area of about 0.55 cm². The receptor wells are filled with phosphate buffered saline solution at pH 7.4 (“PBS”) containing 0.01% sodium azide (a preservative) (the “Receptor Fluid”), that was verified to provide appropriate sink conditions for the diffusing lidocaine throughout the study. The receptor wells of the FDCs are maintained at 32(±1)°C in a stirring dry block with continual stirring of the Receptor Fluid in the receptor well at some 300 rpm using a magnetic stir bar. Donor and receptor chambers are clamped about the skin piece under uniform pressure using a pinch clamp (SS #18 VWR 80073-350).

After the FDCs are assembled, the skin is allowed to hydrate for 20 minutes in contact with the Receptor Fluid. Any FDCs that evidence any leakage during this period are discarded. The integrity and quality of each skin piece is tested prior to application of the test formulations through measurement of the transepidermal electrical resistance (“TEER”). Skin pieces evidencing an excessively low TEER value are discarded and the TEER values of accepted skin pieces are used to guide the distribution of test formulation samples over the skin piece set.

Generally, six (6) replicates of each test formulation are examined, typically in a batch of some 36 FDCs in total. Each solution or gel test formulation is applied using a Nichiryo positive displacement pipette at a dose of 5 µL or 10 µL, corresponding to 9 or 18 mg per cm². For patch formulations, a 10 mm circular piece is cut or punched from each prepared patch formulation, typically some 200 µm in thickness. The patch is applied to the skin exterior and the donor well then applied over the patch, the perimeter of each patch piece being situated under the donor well flange. Dosings of the set of FDCs in a batch are time-staggered, so as to avoid delay in the subsequent disassembly, skin surface cleaning and tape-tripping operations.

At the end of the experiment (30 or 60 mins depending on the study), the FDC is disassembled and the patch is carefully peeled away from the skin surface or, for ointments, gels and solutions, any residual formulation is wiped from the skin exterior with a KimWipe. The skin is further cleaned by applying 200 µL 50:50 water:ethanol, waiting 5 mins, and wiping dry with a Kimwipe. The successive topmost layers of the stratum corneum are removed by three (3) times applying cellophane tape to the skin and then removing the tape. Tape strippings are discarded, the material present in those peripheral layers being considered absorbed only superficially. In certain cases the epidermal and dermal compartments were separated, using mild heating if necessary (to 60° C. for no longer than one minute). The skin sections (or epidermal and dermal sections separately) are placed into 4 mL glass vials. 3 mL of Extraction Fluid is added to each vial and the vials maintained at 32° C. for 24 hours on an orbital shaker. At the end of the extraction period, aliquots of the Extraction Fluid are drawn and analyzed by the verified HPLC-UV method (Example 18).

### Example 23. In Vivo Assessments of Local Anesthesia Effectiveness and Irritation Potential

To assess the effectiveness of a test formulation in causing prompt local anesthesia, the formulation is applied to the forearm of between one and five human volunteer subjects. For a solution, ointment or gel, the test formulation is applied over an approximately 8 cm² area of skin, at a dosing of around 8 mg per cm². In the case of a reservoir patch configuration, the test formulation is introduced into the interior reservoir volume of the reservoir patch and the patch applied to the skin; the formulation contact area is some 3 cm². In the case of a matrix patch, an approximately 2.5×2.5 cm or some 6 cm² area of patch is cut, release liner (if present) removed, and the patch applied to the skin. 45 minutes following formulation application, the test formulation is removed and a sharp object (a Nichiryo positive pipette tip or a 16 gauge syringe needle) is applied to the treated skin area. Each subject rates the level of pain sensation subjectively, that is provides a ‘Pain Score’ or ‘Numbness Score’ on a 1-10 basis, 1 being complete numbness and 10 being no detectable effect. The subject also rates degree of irritation, if present, using a similar scale from 0-10, 0 being non-irritating and 10 being unbearably irritating. For test formulations that prove to be substantially irritating, the experiment is concluded prematurely by prompt removal of the test formulation (preventing then a comparative assessment of numbness).

### Example 24. Short Term Stability Assessment

For assessments of the short term stabilities of transdermal patch formulations, samples of transdermal patches are prepared according to Example 21, heat-sealed in polybags and placed in stability chambers operating at 30° C. ± 2° C. and 65% ± 5% relative humidity (“RH”) and at 40° C. ± 2° C./75% ± 5% RH. At predefined sampling times, such as 1 day, 7 days, 2 weeks, 3 weeks, 4 weeks, 1 month, 2 months, or any time within a range defined by any two of the aforementioned times, pouches are removed from each stability chamber, opened, the patch inside each removed and dissolved in an appropriate volume, e.g. 5 mL, of methanol, and the concentration of lidocaine measured using the verified HPLC-UV method (Example 20).

### Example 25. Long Term Stability Assessment

For assessments of the longer term stabilities of transdermal patch formulations, samples of transdermal patches are prepared according to Example 21, heat-sealed in polybags and placed in stability chambers operating at 25° C. ± 2° C. and 60% ± 5% RH and at 30° C. ± 2° C./65% ± 5% RH. At predefined sampling times, such as 1, 2, 3, 4 weeks, or 1, 2, 3, 4, 5, 6, 7, 8, 9, 10, 11, 12 months, or any time within a range defined by any two of the aforementioned times, pouches are removed from each stability chamber, opened, the patch inside each removed and dissolved in an appropriate volume, e.g. 5 mL, of methanol, and the concentration of lidocaine measured using the verified HPLC-UV method (Example 20).

### Example 26. Methods of Manufacturing Stable Patch Formulations

Transdermal patch formulations that provide suitable chemical and physical stability are conveniently prepared according to the compositions provided in Examples 1 to 19 and the procedure provided in Example 21. A drug product must be manufactured on a consistent basis and subject to exacting quality conditions. To produce a composition as disclosed herein, preparation should be in line with the disclosed preparative procedures. Due consideration should be given to one or more of the following: (i) use of a pharmaceutical grade of each excipient, (ii) adherence to the methods of the present disclosure, (iii) achieving a substantially homogeneous solution following combination of all ingredients, (iv) reducing premature evaporative losses, (v) substantial removal of air bubbles prior to film formation, (vi) deposition of films of uniform thickness on the chosen substrate, (vii) limited introduction of contaminants or chemical changes in the formulation by the spreading apparatus, (viii) substantially uniform and controlled drying conditions.

### Example 27. Kit for Use in Alleviating Needle Pain

This non-limiting embodiment relates to a kit, and its use, for reduction, ameliorating or eliminating needle pain (e.g., pain associated with needle sticks or skin punctures). In several embodiments, the kit comprises (i) at least one pre-dosed tube of a fast onset lidocaine formulation (for example those disclosed herein) (ii) a self-adhesive gel ring (or other shape with an open central region), and (iii) a waterproof occlusive dressing or backing film.

In several embodiments, the pre-dosed lidocaine is a lidocaine gel. In several embodiments, the lidocaine is clear, or substantially clear, for example to aid in visual identification of an injection site. In several embodiments, the pre-dosed lidocaine is dosed for a particular patient, for example a pediatric subject, an adult subject etc. In several embodiments, different kits are provided, for example with higher doses provided for patients with particular sensitivities to needle sticks or other skin punctures. In several embodiments, at least a second dose of lidocaine is provided in a kit, for example if additional anesthesia is needed for a subject. In several embodiments, the pre-dosed amount of lidocaine ensures an appropriate dose for a given patient to maximize efficacy and/or minimize risk for overdose.

In several embodiments, the lidocaine formulation is advantageous in that it is believed to be safer than available prescriptions containing tetracaine or prilocaine (both increase risk of potential methemoglobinemia). In several embodiments, the use of a lidocaine gel provides advantageous antimicrobial properties to reduce risk of infection at a site of injection. In several embodiments, the gel is clear, which aids in visualization of skin by a medical provider for monitoring of potential adverse reaction.

In several embodiments, the ring is silicone or other relatively soft and pliable material. Any biocompatible, flexible material is suitable, so long as it adheres to the target site, even during motion of the tissue surrounding the target site. In several embodiments, the flexibility and conformance of the ring (or other shape with an open central region) keeps medications localized to intended area. As such, in several embodiments, the use of the ring (or other shape) minimizes the need for excessive medication, thereby enhancing safety for patients. In several embodiments, the ring further comprises a dye or other colorant that temporarily marks the skin of the subject in the shape of the ring (for example to identify the target site for injection). Alternatively, the kit may further comprise a skin marking pen.

In several embodiments, the occlusive dressing or backing film is transparent, or substantially so, to facilitate a clear visual view of the target area. This allows, in several embodiments, a medical provider to view the skin throughout the duration of a procedure (from application of anesthetic to completion of injection) to detect possible adverse effects or reactions. However, the occlusive dressing or backing film does not necessarily need to be transparent.

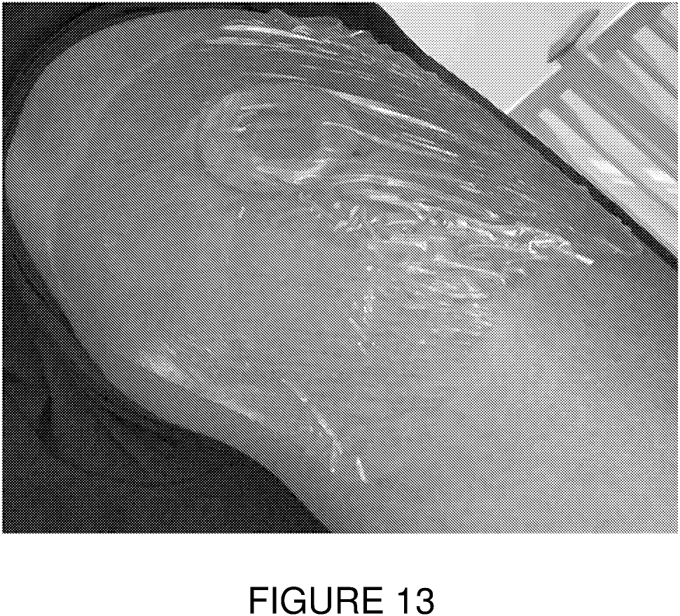

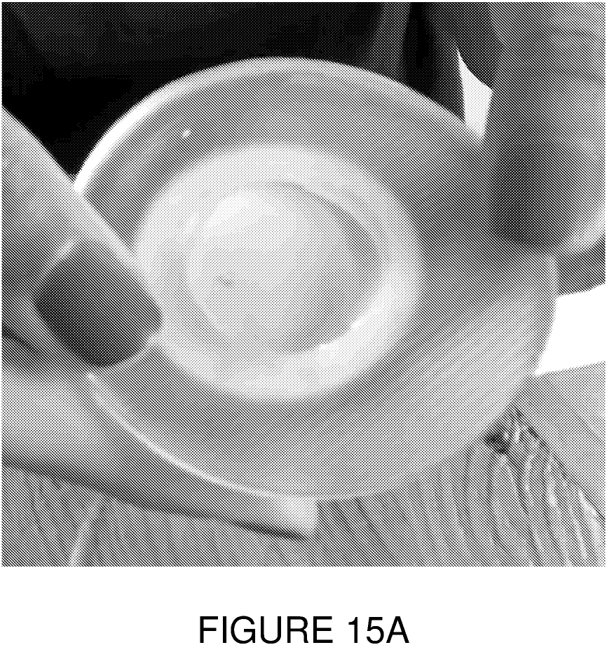

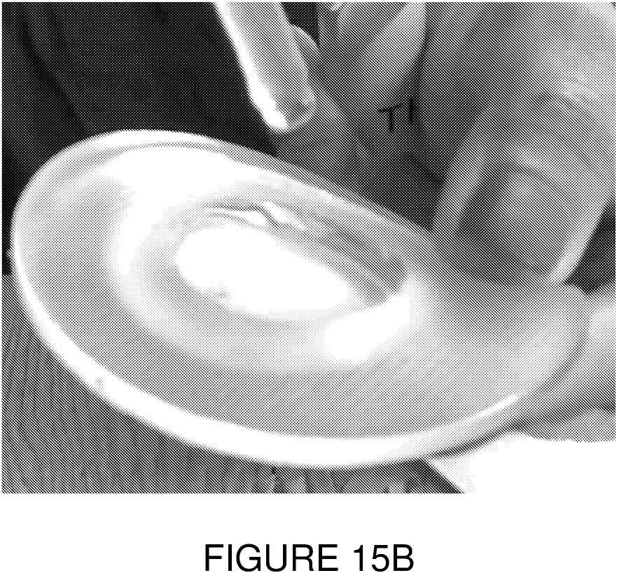

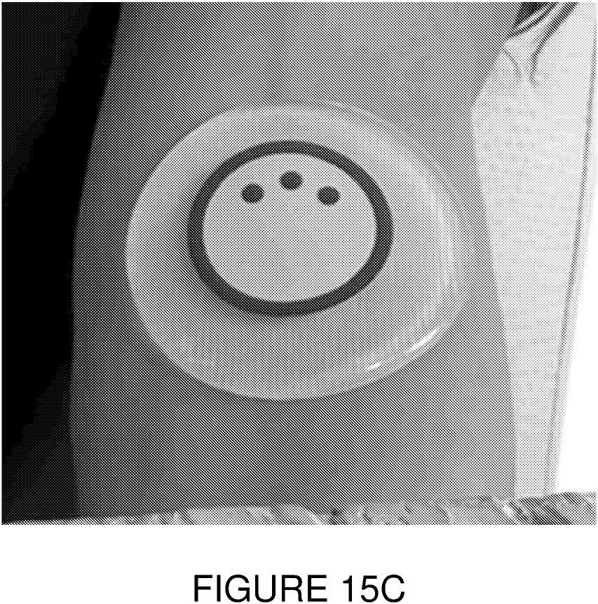

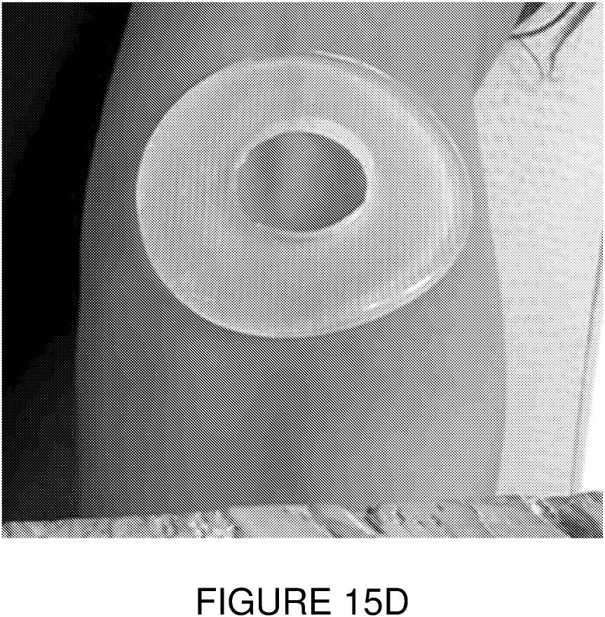

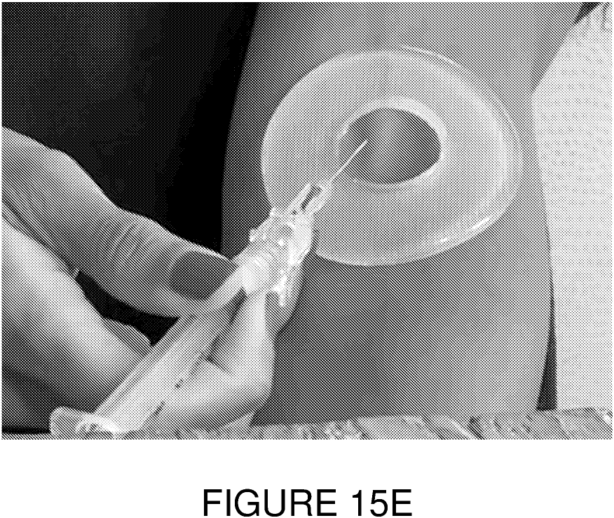

In use, according to several embodiments, a silicone gel ring is placed onto clean dry skin. Gel (e.g., a lidocaine formulation as disclosed herein) is dispensed from the predetermined dosing tube and is placed into center of the ring. The occlusive dressing or backing film is placed atop, or has been placed on the center of ring previously (e.g. during manufacture). FIG. 13 shows a use of the kit according to several embodiments with a ring applied to the skin of a patient at a target site and covered with an occlusive dressing (such as TEGADERM®). FIGS. 15A-E shows additional embodiments of a silicone gel ring with backing film.

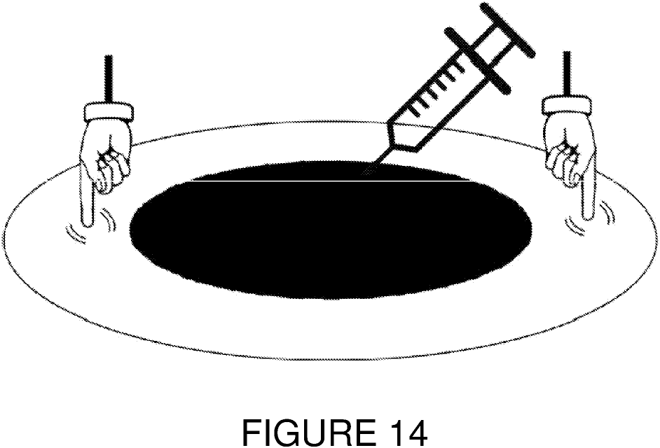

The system is left in place for 30-90 minutes. At the time of the needle procedure, the occlusive dressing or backing film is removed and the gel ring is left on the skin in order to delineate the site of anesthetized skin (FIGS. 15C-D). In several embodiments, downward pressure, around the anesthetized site in one or more places, is applied in order to stimulate local nerves that sense pressure and/or vibration, which in several embodiments, further alleviates the sensations of pain from a needle stick or skin puncture (see, e.g., FIGS. 14 and 15E). If the desired degree of anesthesia is not met, the area can be re-dosed and/or re-occluded for an additional period of time. At such time that anesthesia is reached, the lidocaine gel may be removed by the medical provider with an alcohol pad (or by tissue or other means) and the injection can then be administered. Post-injection, the gel ring is removed and discarded.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

Any titles or subheadings used herein are for organization purposes and should not be used to limit the scope of embodiments disclosed herein. Moreover, a listing of one ingredient under one heading does not preclude it also being considered under another heading. For example, an ingredient listed under the heading of “Preservative” may also function in some embodiments as an anti-oxidant, despite not being listed under the heading “Anti-oxidant”.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

It is understood that the examples and embodiments described herein are for illustrative purposes only. Various modifications or changes in light thereof will be suggested to persons skilled in the art and are to be included within the spirit and purview of this application and the scope of the appended claims. All publications, patents, and patent applications cited herein are hereby incorporated by reference in their entirety for all purposes.

### REFERENCES

Potts RO & Guy RH. “A Predictive Algorithm for Skin Permeability: The Effects of Molecular Size and Hydrogen Bond Activity” Pharmaceutical Research (1995) 12:1628-1633
Ostrenga J et al. “Significance of vehicle composition I: Relationship between topical vehicle composition, skin penetrability, and clinical efficacy” J. Pharm. Sci. (1971) 60(8):1175-1179
Smith EW, Maibach HI: Percutaneous Penetration Enhancers: CRC Press 1995.
Osborne DW & Henke JJ. “Skin penetration enhancers cited in the technical literature” Pharmaceutical Technology. (1997) 21(11):58-66
Thong HY et al. “Percutaneous Penetration Enhancers: An Overview” Skin Pharmacology and Physiology. (2007) 20(6):272-282
Ahad A et al. “Chemical penetration enhancers: a patent review” Expert Opinion on Therapeutic Patents. (2009) 19(7):969-988
Lane ME “Skin penetration enhancer” Int. J. Pharmaceutics. (2013) 447(1-2):12-21
Ita KB: Chemical Penetration Enhancers for Transdermal Drug Delivery - Success and Challenges. Curr Drug Deliv 2015, 12:645-651.
Lopes LB et al. “Chemical penetration enhancers” Therapeutic Delivery. (2015) 6(9):1053-1061
Dragicevic N, Maibach HI (Ed): Percutaneous Penetration Enhancers: Chemical Methods in Penetration Enhancement (Modification of the Stratum Corneum): Springer; 2016.
Sweet PT et al. “Duration of intradermal anaesthesia with mixtures of bupivacaine and lidocaine” Canadian Anaesthetists’ Society Journal. (1982) 29:481-483
Costa CG et al. “Onset and duration periods of articaine and lidocaine on maxillary infiltration” Noteworthy Abstracts of the Current Literature. (2005) 94(4):P381
Collins JB et al. “Onset and duration of intradermal mixtures of bupivacaine and lidocaine with epinephrine” Can. J. Plast. Surg. (2013) 21(1):51-3
Kreilgaard M: Dermal pharmacokinetics of microemulsion formulations determined by in vivo microdialysis. Pharm Res 2001,18:367-373.
Yamamoto S et al. “Prediction of Human Pharmacokinetic Profile After Transdermal Drug Application Using Excised Human Skin” J. Pharm. Sci. (2017) 106(9):2787-2794
Franz TJ “Percutaneous Absorption. On the Relevance of in Vitro Data” J. Investigative Dermatology. (1975) 64(3):190-195
Hind H: Method for treating nerve injury pain associated with shingles (herpes-zoster and post-herpetic neuralgia) by topical application of lidocaine. U.S. Pat. 5,411,738, 1995.
Ono M, Akazawa M, Seki M, Iwamoto K, Konishi R: External preparation for application to the skin containing lidocaine. U.S. Pat. 5,827,529, 1998.
Luo EC, Gricenko NT, Hsu T-M: Transdermal and topical administration of local anesthetic agents using basic enhancers. U.S. Pat. 6,673,363, 2004.
Buyuktimkin N, Buyuktimkin S, Singh J, Newsam JM, King-Smith D, Kisak ET, Galer BS, Desai T, Riley C: Pharmaceutical Formulations and Methods of Use. U.S. Provisional Pat. Application 61/237,252 (Aug. 26, 2009) PCT Patent Application WO/2011/028629 (Mar. 10, 2011), 2011.
Shantha TR, Shantha J, Shantha E, Shantha L: Shantha, Erica. U.S. Pat. 7,883,487, 2011.
Ciullo J: Lidocaine patch and methods of use thereof. US20130184351A1, 2013.
Kubo J, Tsuru S, Tsurushima K, Yamasoto S: Lidocaine-containing hydrogel patch. U.S. Pat. 8,920,831, 2014.
Zhang J, Lippert R: Heat assisted lidocaine and tetracaine for transdermal analgesia. U.S. Pat. 9,186,334, 2015.
Mori T, Saida N: Non-aqueous patch. U.S. Pat. 9,925,264, 2018.
Williams AC, Barry BW: Penetration enhancers. Adv Drug Deliv Rev 2004, 56:603-618.
External Analgesic Drug Products For Over-The Counter Human Use; Tentative Final Monograph. In Federal Register. Edited by. Washington, DC; 1983. [Administration FRDoHaHSFaD (Series Editor), vol Vol. 48.]
Cousins MJ, Bridenbaugh PO, Carr DB, Horlocker TT (Ed): Cousins and Bridenbaugh’s Neural Blockade in Clinical Anesthesia and Pain Medicine (Fourth Edition): Lippincott Williams & Wilkins; 2009.
Drug Facts and Comparisons 2017: Lippincott Williams & Wilkins; 2016.
Sandbom E: Pharmaceutical solutions comprising dimethyl sulfoxide. U.S. Pat. 4,652,557, 1988.
Kisak E, Singh J: Diclofenac topical formulation. U.S. Pat. 8,252,838 2012.
Kisak E, Singh J: Diclofenac topical formulation. U.S. Pat. 8,563,613, 2013.
Kisak E, Singh J: Diclofenac topical formulation. U.S. Pat. 8,871,809, 2014.
Kisak E, Singh J: Diclofenac topical formulation. U.S. Pat. 9,066,913, 2015.
Kisak E, Singh J: Diclofenac topical formulation. U.S. Pat. 9,101,591, 2015.
Buyuktimkin S, Buyuktimkin N, Kisak E, Singh J, Newsam JM, King-Smith D, Galer BS: Foamable formulation. U.S. Pat. 9,107,823, 2015.

What is claimed is:

1. A topical formulation comprising:
lidocaine present in an amount of 1% to 10% or about 1% to about 10% w/w;
limonene present in an amount of 5% to 20% or about 5% to about 20% w/w; and
at least one C2-C4 monohydric alcohol present in an amount of 40% to 60% or about 40% to about 60% w/w.

2. The topical formulation of claim 1, wherein the at least one C2-C4 monohydric alcohol comprises ethanol.

3. The topical formulation of claim 1, wherein the at least one C2-C4 monohydric alcohol is ethanol.

4. The topical formulation of claim 1, wherein the lidocaine is present in an amount of 4% or about 4% w/w.

5. The topical formulation of claim 4, wherein the limonene is present in an amount of 10% or about 10% w/w.

6. The topical formulation of claim 1, wherein the at least one C2-C4 monohydric alcohol is present in an amount of 51% or about 51% w/w.

7. The topical formulation of claim 1, further comprising at least one isopropyl ester of a C14-C18 fatty acid.

8. The topical formulation of claim 7, wherein the at least one isopropyl ester of a C14-C18 fatty acid is present in an amount of 10% to 30% or about 10% to about 30% w/w.

9. The topical formulation of claim 7, wherein the at least one isopropyl ester of a C14-C18 fatty acid comprises isopropyl palmitate or isopropyl myristate, or both.

10. The topical formulation of claim 9, wherein the isopropyl palmitate and the isopropyl myristate are each present in an amount of 5% to 15% or about 5% to about 15% w/w.

11. The topical formulation of claim 9, wherein the isopropyl palmitate is present in an amount of 7% or about 7% w/w.

12. The topical formulation of claim 9, wherein the isopropyl myristate is present in an amount of 11% or about 11% w/w.

13. The topical formulation of claim 9, wherein the lidocaine is present in an amount of 4% or about 4% w/w, the limonene is present in an amount of 10% or about 10% w/w, the at least one C2-C4 monohydric alcohol is present in an amount of 51% or about 51% w/w, the isopropyl palmitate is present in an amount of 7% or about 7% w/w, and the isopropyl myristate is present in an amount of 11% or about 11% w/w.

14. The topical formulation of claim 1, further comprising at least one monohydric C14-C20 alcohol.

15. The topical formulation of claim 14, wherein the at least one monohydric C14-C20 alcohol is present in an amount of 5% to 15% or about 5% to about 15% w/w.

16. The topical formulation of claim 14, wherein the at least one monohydric C14-C20 alcohol comprises isostearyl alcohol.

17. The topical formulation of claim 14, wherein the at least one monohydric C14-C20 alcohol is isostearyl alcohol.

18. The topical formulation of claim 17, wherein the isostearyl alcohol is present in an amount of 10% or about 10% w/w.

19. The topical formulation of claim 18, wherein the lidocaine is present in an amount of 4% or about 4% w/w, the limonene is present in an amount of 10% or about 10% w/w, the at least one C2-C4 monohydric alcohol is present in an amount of 51% or about 51% w/w, the isopropyl palmitate is present in an amount of 7% or about 7% w/w, the isopropyl myristate is present in an amount of 11% or about 11% w/w, and the at least one monohydric C14-C20 alcohol is present in an amount of 10% or about 10% w/w.

20. The topical formulation of claim 1, further comprising at least one polyalkylene glycol alkyl ether.

21. The topical formulation of claim 20, wherein the at least one polyalkylene glycol alkyl ether is present in an amount of 0% to 10% or about 0% to about 10%.

22. The topical formulation of claim 20, wherein the at least one polyalkylene glycol alkyl ether comprises polyethylene glycol dodecyl ether.

23. The topical formulation of claim 20, wherein the at least one polyalkylene glycol alkyl ether is polyethylene glycol dodecyl ether.

24. The topical formulation of claim 23, wherein the polyethylene glycol dodecyl ether is present in an amount of 5% or about 5% w/w.

25. The topical formulation of claim 24, wherein the lidocaine is present in an amount of 4% or about 4% w/w, the limonene is present in an amount of 10% or about 10% w/w, the at least one C2-C4 monohydric alcohol is present in an amount of 51% or about 51% w/w, the isopropyl palmitate is present in an amount of 7% or about 7% w/w, the isopropyl myristate is present in an amount of 11% or about 11% w/w, the at least one monohydric C14-C20 alcohol is present in an amount of 10% or about 10% w/w, and the at least one polyalkylene glycol alkyl ether is present in an amount of 5% or about 5% w/w.

26. The topical formulation of claim 1, further comprising a cellulosic thickening agent.

27. The topical formulation of claim 26, wherein the cellulosic thickening agent is present in an amount of 0% to 5% or about 0% to about 5%.

28. The topical formulation of claim 26, wherein the cellulosic thickening agent comprises hydroxypropyl cellulose.

29. The topical formulation of claim 28, wherein the hydroxypropyl cellulose is present in an amount of 2% or about 2%.

30. The topical formulation of claim 29, wherein the lidocaine is present in an amount of 4% or about 4% w/w, the limonene is present in an amount of 10% or about 10% w/w, the at least one C2-C4 monohydric alcohol is present in an amount of 51% or about 51% w/w, the isopropyl palmitate is present in an amount of 7% or about 7% w/w, the isopropyl myristate is present in an amount of 11% or about 11% w/w, the at least one monohydric C14-C20 alcohol is present in an amount of 10% or about 10% w/w, the at least one polyalkylene glycol alkyl ether is present in an amount of 5% or about 5% w/w, and the cellulosic thickening agent is present in an amount of 2% or about 2%.

31. A topical formulation comprising:
lidocaine present in an amount of 1% to 10% or about 1% to about 10% w/w;
a pressure sensitive adhesive present in an amount of 70% to 90% or about 70% to about 90% w/w;
a molecular penetration enhancer present in an amount of 0% to 5% or about 0% to about 5% w/w; and
at least one polyalkylene glycol alkyl ether present in an amount of 0% to 5% or about 0% to about 5% w/w.

32. The topical formulation of claim 31, wherein the lidocaine is present in an amount of 4% or about 4% w/w.

33. The topical formulation of claim 31, wherein the pressure sensitive adhesive is present in an amount of 83% or about 83% w/w.

34. The topical formulation of claim 31, wherein the pressure sensitive adhesive comprises DURO-TAK 87-4098.

35. The topical formulation of claim 31, wherein the molecular penetration enhancer is present in an amount of 3% or about 3% w/w.

36. The topical formulation of claim 31, wherein the molecular penetration enhancer comprises levulinic acid.

37. The topical formulation of claim 31, wherein the at least one polyalkylene glycol alkyl ether is present in an amount of 2% or about 2%.

38. The topical formulation of claim 31, wherein the at least one polyalkylene glycol alkyl ether comprises polyethylene glycol dodecyl ether.

39. The topical formulation of claim 31, wherein the at least one polyalkylene glycol alkyl ether is polyethylene glycol dodecyl ether.

40. The topical formulation of claim 31, further comprising at least one isopropyl ester of a C14-C18 fatty acid.

41. The topical formulation of claim 40, wherein the at least one isopropyl ester of a C14-C18 fatty acid is present in an amount of 5% to 10% or about 5% to about 10% w/w.

42. The topical formulation of claim 40, wherein the at least one isopropyl ester of a C14-C18 fatty acid comprises isopropyl palmitate or isopropyl myristate, or both.

43. The topical formulation of claim 42, wherein the isopropyl palmitate and the isopropyl myristate are each present in an amount of 2.5% to 5% or about 2.5% to about 5% w/w.

44. The topical formulation of claim 42, wherein the isopropyl palmitate or the isopropyl myristate, or both, are present in an amount of 4% or about 4% w/w.

45. The topical formulation of claim 44, wherein the lidocaine is present in an amount of 4% or about 4% w/w, the pressure sensitive adhesive is present in an amount of 83% or about 83% w/w, the molecular penetration enhancer is present in an amount of 3% or about 3% w/w, the polyalkylene glycol alkyl ether is present in an amount of 2% or about 2% w/w, the isopropyl palmitate is present in an amount of 4% or about 4% w/w, and the isopropyl myristate is present in an amount of 4% or about 4% w/w.

46. A method of preventing local pain in a subject, comprising the topical administration to said subject of a therapeutically effective amount of the topical formulation of any one of claims 1-45.

47. A device suitable for providing rapid local anesthesia comprising an annulus of pressure sensitive adhesive, within the inner circumference of which is provided the topical formulation of any one of claims 1-45.

48. The device of claim 47, further comprising a backing film on which the topical formulation is applied, wherein the backing film covers the inner circumference of the annulus of pressure sensitive adhesive.

49. The device of claim 48, wherein the backing film is intended to be removed after application of the device such that the inner circumference of the annulus of pressure sensitive adhesive is exposed to allow access with a needle.

50. The device of claim 47, wherein the topical formulation is provided as a single dose packet.

51. A kit for use in anesthetizing a target area of skin of a subject, the kit comprising:
(i) a predetermined dose of a topical anesthetic; and
(ii) an annulus of pressure sensitive adhesive comprising an outer perimeter that adheres to the skin of the subject, and an open center region in which the skin is exposed.

52. The kit of claim 51, further comprising (iii) an occlusive, waterproof dressing or a backing film to cover the open center region and target area.

53. The kit of claim 51, wherein the topical anesthetic comprises a topical formulation of any one of claims 1-45.

54. The kit of claim 51, wherein the topical anesthetic comprises lidocaine and wherein the lidocaine is optionally in gel format.

55. The kit of claim 51, wherein the annulus of pressure sensitive adhesive comprises a silicone ring.

56. The kit of claim 51, wherein the predetermined dose of the topical anesthetic is provided as a single dose packet.

57. A topical formulation comprising
lidocaine,
wherein the lidocaine is present in an amount of 1% to 10% or about 1% to about 10% w/w;
limonene,
wherein the limonene is present in an amount of 5% to 20% or about 5% to about 20% w/w;
DMSO,
wherein the DMSO is present in an amount of 40% to 70% or about 40% to about 70% w/w; and
at least one C2-C4 monohydric alcohol,
wherein the at least one C2-C4 monohydric alcohol comprises ethanol, and wherein the ethanol is present in an amount of 10% to 50% or about 10% to 50% w/w.

58. The topical formulation of claim 57, wherein
(i) lidocaine is in an amount of 4% or about 4%;
(ii) limonene is in an amount of 10% or about 10%;
(iii) DMSO is in an amount of 66% or about 66%; and
(iv) ethanol is in an amount of 20% or about 20%.

59. The topical formulation of claim 57, wherein
(i) lidocaine is in an amount of 4% or about 4%;
(ii) limonene is in an amount of 10% or about 10%;
(iii) DMSO is in an amount of 45% or about 45%; and
(iv) ethanol is in an amount of 41% or about 41%.

60. The topical formulation of claim 57, further comprising at least one isopropyl ester of a C14-C18 fatty acid.

61. The topical formulation of claim 59, wherein the at least one isopropyl ester of a C14-C18 fatty acid comprises isopropyl myristate, or isopropyl palmitate, or both.

62. The topical formulation of claim 61, wherein isopropyl myristate and isopropyl palmitate are each in an amount of 0% to 15% or about 0% to about 15%.

63. The topical formulation of claim 62, wherein
(i) lidocaine is in an amount of 4% or about 4%;
(ii) limonene is in an amount of 10% or about 10%;
(iii) DMSO is in an amount of 45% or about 45%;
(iv) ethanol is in an amount of 31% or about 31%;
(v) isopropyl myristate is in an amount of 10% or about 10%; and
(vi) isopropyl palmitate is in an amount of 0% or about 0%.

64. The topical formulation of claim 62, wherein
(i) lidocaine is in an amount of 4% or about 4%;
(ii) limonene is in an amount of 10% or about 10%;
(iii) DMSO is in an amount of 45% or about 45%;
(iv) ethanol is in an amount of 31% or about 31%;
(v) isopropyl myristate is in an amount of 0% or about 0%; and
(vi) isopropyl palmitate is in an amount of 10% or about 10%.

65. The topical formulation of claim 60, further comprising at least one monohydric C14-C20 alcohol.

66. The topical formulation of claim 65, wherein the at least one monohydric C14-C20 alcohol comprises isostearyl alcohol.

67. The topical formulation of any one of claims 57-66, further comprising polyethylene glycol dodecyl ether.

68. A method of preventing local pain in a subject, said method comprising the topical administration to said subject of a therapeutically effective amount of the formulation of any one of claims 57-66.

69. A device suitable for providing rapid local anesthesia comprising an annulus of pressure-sensitive adhesive, within the inner circumference of which is provided the topical formulation of any one of claims 57-66.

70. A topical formulation comprising lidocaine, at least one C2-C4 monohydric alcohol, limonene, at least one isopropyl ester of a C14-C18 fatty acid, and at least one monohydric C14-C20 alcohol.

71. The topical formulation of claim 70, wherein the at least one C2-C4 monohydric alcohol comprises ethanol.

72. The topical formulation of claim 70, wherein the at least one isopropyl ester of the C14-C18 fatty acid comprises isopropyl myristate.

73. The topical formulation of claim 70, wherein the at least one isopropyl ester of the C14-C18 fatty acid comprises isopropyl palmitate.

74. The topical formulation of claim 70, wherein the at least one isopropyl ester of the C14-C18 fatty acid comprises both isopropyl myristate and isopropyl palmitate.

75. The topical formulation of claim 70, wherein the at least one monohydric C14-C20 alcohol comprises isostearyl alcohol.

76. The topical formulation of any one of claims 70-75, further comprising polyethylene glycol dodecyl ether.

77. A method of preventing local pain in a subject, said method comprising the topical administration to said subject of a therapeutically effective amount of the formulation of any one of claims 70-76.

78. A device suitable for providing rapid local anesthesia comprising an annulus of pressure-sensitive adhesive, within the inner circumference of which is provided the topical formulation of any one of claims 70-76.

79. A formulation suitable for topical administration, comprising:
(i) at least one active agent;
(ii) at least one C2-C4 monohydric alcohol;
(iii) limonene;
(iv) the isopropyl esters of at least two C14-C18 fatty acids; and
(v) at least one monohydric C14-C20 alcohol.

80. A kit for use in anesthetizing a target area of tissue, the kit comprising:
(i) a predetermined dose of a topical anesthetic;
(ii) a self-adhesive patch comprising an outer perimeter which adheres to the skin and an open center region in which skin is exposed;
(iii) an occlusive, waterproof dressing to cover the patch and target area.

81. The kit according to claim 80, wherein the topical anesthetic comprises a formulation according to any one of claims 55-64 or 68-74.

82. The kit according to claim 80 or 81, wherein the topical anesthetic comprises lidocaine, wherein the lidocaine is optionally in gel format.

83. The kit according to any one of claims 80-82, wherein the self-adhesive patch comprises a silicone ring.
